(19) United States
(12) Patent Application Publication
(10) Pub. No.: US 2023/0279373 A1
(43) Pub. Date: Sep. 7, 2023

(54) NOVEL CRISPR ENZYMES, METHODS, SYSTEMS AND USES THEREOF

(71) Applicant: Beam Therapeutics Inc., Cambridge, MA (US)

(72) Inventors: Bernd ZETSCHE, Cambridge, MA (US); Luis BARRERA, Cambridge, MA (US)

(21) Appl. No.: 17/641,356

(22) PCT Filed: Sep. 9, 2020
(86) PCT No.: PCT/US2020/049890

§ 371 Date: Mar. 8, 2022

Related U.S. Application Data
(60) Provisional application No. 62/897,929, filed on Sep. 9, 2019.
(60) Provisional application No. 62/907,238, filed on Sep. 27, 2019.

(51) Int. Cl. C12N 9/22; C12N 9/78; C12N 15/86; C12N 15/11

(52) U.S. Cl. CPC C12N 9/22; C12N 9/78; C12N 15/86; C12N 15/11; C12N 2310/20; C12N 2750/14141; C12Y 305/04005; C12Y 305/04004

(57) ABSTRACT

The present invention provides novel systems, methods and compositions for making and using a recombinantly engineered novel Cas9 optimized for human cells, for nucleic acid targeting and manipulation. The present invention is based on the discovery of a novel Cas9 species from Lachnospira bacterium that was codon-optimized and recombinantly produced for use in human ceils. In some embodiments, the novel Cas9 can be used in a base editor. In some embodiments, the novel engineered Cas9 is used to treat human diseases.

### CROSS-REFERENCE TO RELATED APPLICATIONS

This application claims benefit of, and priority to, U.S. Ser. No. 62/897,929 filed on Sep. 9, 2019 and U.S. Ser. No. 62/907,238 filed on Sep. 27, 2019, the contents of each of which are incorporated herein.

### BACKGROUND

Enzymes from the prokaryotic Clustered, Regularly Interspaced Short Palindromic Repeats (CRISPR) and CRISPR-associated protein (CRISPR-Cas) systems have been harnessed as reprogrammable and highly specific genome editing tools for use in eukaryotes. Besides genome editing and cleavage, CRISPR-Cas9 can be used to localize effector molecules to specific sites on the genome, allowing genetic and epigenetic regulation and transcriptional modulation through a variety of mechanisms.

However, diverse genomes and genomic targets require a variety of tools for effective genetic engineering, and there remains a need to expand the CRISPR toolbox through the discovery and engineering of novel Cas proteins that can recognize and target diverse sequences.

While CRISPR-Cas9 systems can be used to knock out a gene or modify the expression of a gene, certain kind of gene editing requires precise modifications to the target gene, such as editing a single base within the gene. Such precise modifications remain a challenge and requires a diverse gene editing toolkit to effectuate precise genomic modifications in a wide variety of target genes.

### SUMMARY OF THE INVENTION

The identification of novel Cas9 enzymes with specificity for unique protospacer adjacent motifs (PAM) allows for the expansion of the available tools for gene editing. The present invention provides, among other things, an engineered, non-naturally occurring Cas9 protein modified from Lachnospira bacteria. The present invention is based, in part, on the surprising discovery that a novel Cas9 discovered from Lachnospira bacteria, can be engineered for expression in eukaryotic cells (e.g., human, plant, etc.), and which recognizes a specific PAM sequence defined by 5′-NNGNG-3′. The examples provided herewith show use of this engineered, non-naturally Cas9 in human cells to target various genomic sites.

In one aspect, an engineered, non-naturally occurring Cas9 protein modified from Lachnospira Cas9 is provided herein.

In some embodiments, the Cas9 protein has at least 80% sequence identity to

(SEQ ID NO: 1)
MSVNVGLDIGIASVGVAVVDSESGEILEAVSDLFESAEANQNVDRRGFR
QSRRLKRRQYNRIHDFMKLWEEFGFVKPENINLNTVGLRVKSLTEQVTL
DELYVILLSELKHRGISYLEDSEEVDGGSEYKEGLRINQRELQSKYPCE
IQLERLKIYGRYRGNFTVEIDGEKVGLSNVFTTGAYRKEIQQLLSIQKT
YQSKLTDDFINKYLEIFDRKRQYYVGPGNEKSRTDYGRYTTKKDAEGNY
ITDENIFEKLIGKCSIYPEEMRAAGASYTAQEFNLLNDLNNLTIGGRKI
EEEEKRAIIETIKSSKVVNVEKIICKVTGEDAETITGARIDKDDKRIYH
SFECYRKLKKALETIEVKIEEYSREELDELARILTLNTEREGILGELEK
SFLDLGEEVIDCVIDFRRKNGPLFSKWQSFSLRLMNDIIPDMYEQPKEQ
MTLLTEMGLMKSKKEIFKGMKYIPENVMRDDIYNPVVVRSVRIAVRALN
AVIKKYGEIDKVVIEMPRDRNTEEQKKRIDAENKRNREELPGIEKRILE
EYGIKITSAHYRNHKQLGLKLKLWNEQGGICPYSGKTIDLERLLQNAGD
YEVDHIIPLSISLDDSRNNKVLVYASENQKKGNQTPYAYLSSVQREWGW
EQYRHYVLSDLKKKKISSKKIENYLFMKDISKIDVVKGFIQRNLNDTRY
ASKVVLNTLESFFKANEKETKVSVIRGSFTSLMRKNLKLDKSREESYAH
HAVDALLIAYSKMGYDSYHKLQGEFIDFETGEILDSRMWETNLEPDILK
GYLYGRKWSEIRENIKIAESRVKYWHMTNKKCNRSLCNQTLYGTRTYDG
KIYQIKKIKDIRTPEGLKTFKDLVDKNKGDHLLMARNDPKTYEQILQIY
RDYSDAKNPFLQYEMETGDCIRKYSKKHNGSRIVSLKYHDGEVNSCIDV
SHKYGFEKGSQKVVLMSLNPYRMDVYKNCNDGKYYLIGLKQSDIKCEGR
HYVIDEEKYAKVLVNEKMIQPGQSRKDLPDLGYEFVMSFYKNEIIQYEK
DGKFYKERFLSRTKPASRNYIETKPVDKPNFEKRHQIGLAKTTFIRKIR
TDILGNEYNCDREKFSSIC.

In some embodiments, the Cas9 protein comprises an amino acid sequence that is at least 85% at least 90° %, at least 92%, at least 95%, at least 96%, at least 97%, at least 980%, or at least 99% identical to SEQ ID NO: 1.

In some embodiments, the amino acid sequence of the Cas9 protein comprises at least one, at least two, at least three, at least four, at least five, at least six, at least seven, at least eight, at least nine, or at least 10 mutations in SEQ ID NO: 1.

In some embodiments, the mutation is an amino acid substitution.

In some embodiments, the Cas9 protein has nickase activity.

In some embodiments, the amino acid sequence comprises at least one mutation in an amino acid residue selected from amino acids 7, 593, and/or 616 of SEQ ID NO: 1.

In some embodiments, the at least one mutation in amino acid residue is D8A, H593A, and/or N616A.

In some embodiments, the at least one mutation results in an inactive Cas9 (dCas9).

In some embodiments, the Cas9 protein comprises at least one amino acid mutation in PAM Interacting, HNH and/or RuvC domain.

In some embodiments, the Cas9 protein further comprises a nuclear localization sequence (NLS) and/or a FLAG, HIS or HA tag.

In one aspect, provided herein is an engineered, non-naturally occurring Cas9 fusion protein comprising a Cas9 protein having at least 80% identity to SEQ ID NO: 1, and wherein the Cas9 protein is fused to a histone demethylase, a transcriptional activator, or to a deaminase.

In some embodiments, the Cas9 protein is fused to a cytosine deaminase or to an adenosine deaminase.

In some embodiments, the Cas9 protein recognizes a PAM sequence comprising 5′-NNGNG-3′.

In some embodiments, a nucleic acid encoding the Cas9 protein is provided.

In some embodiments, the nucleic acid is codon-optimized for expression in mammalian cells.

In some embodiments, the nucleic acid is codon-optimized for expression in human cells.

In some embodiments, a eukaryotic cell comprising the Cas9 protein is provided.

In some embodiments, the cell is a human cell. In some embodiments, the cell is a plant cell.

In one aspect, a method of cleaving a target nucleic acid in a eukaryotic cell is provided comprising: contacting the cell with a Cas9 as described herein, and an RNA guide or a nucleic acid encoding the RNA guide, wherein the RNA guide comprises a direct repeat sequence and a spacer sequence capable of hybridizing to the target nucleic acid, and wherein the Cas9 protein is capable of binding to the RNA guide and of causing a break in the target nucleic acid sequence complementary to the RNA guide.

In one aspect, a method of altering expression of a target nucleic acid in a eukaryotic cell is provided comprising: contacting the cell with a Cas9 as described herein, and an RNA guide or a nucleic acid encoding the RNA guide, wherein the RNA guide comprises a direct repeat sequence and a spacer sequence capable of hybridizing to the target nucleic acid, and wherein the Cas9 protein is capable of binding to the RNA guide and of causing a break in the target nucleic acid sequence complementary to the RNA guide.

In one aspect, a method of altering expression of a target nucleic acid in a eukaryotic cell is provided comprising: contacting the cell with a Cas9 as described herein, and an RNA guide or a nucleic acid encoding the RNA guide, wherein the RNA guide comprises a direct repeat sequence and a spacer sequence capable of hybridizing to the target nucleic acid, and wherein the Cas9 protein is capable of binding to the RNA guide and editing the target nucleic acid sequence complementary to the RNA guide.

In one aspect, a method of modifying a target nucleic acid in a eukaryotic cell is provided comprising: contacting the cell with a Cas9 as described herein, and an RNA guide or a nucleic acid encoding the RNA guide, wherein the RNA guide comprises a direct repeat sequence and a spacer sequence capable of hybridizing to the target nucleic acid, and wherein the Cas9 protein is capable of binding to the RNA guide and editing the target nucleic acid sequence complementary to the RNA guide.

In some embodiments, the Cas9 protein is an inactive Cas9 (dCas9).

In some embodiments, the dCas9 is fused to a deaminase.

In some embodiments, the RNA guide comprises a crRNA and a tracrRNA.

In some embodiments, the crRNA comprises a guide sequence of between about 16 and 26 nucleotides long.

In some embodiments, the crRNA comprises a guide sequence between 18 and 24 nucleotides long.

In some embodiments, the crRNA comprises a direct repeat (DR) sequence of between about 16 and 26 nucleotides long.

In some embodiments, the crRNA comprises a 22 nucleotide guide sequence and a 22 nucleotide direct repeat (DR) sequence.

In some embodiments, the crRNA comprises a DR sequence comprising a sequence having at least about 80% identity to

(SEQ ID NO: 3)
AUUUUAGUUCCUGGAUAAUUCAAGUUAGUGUAAAAC.

In some embodiments, the crRNA comprises a DR sequence comprising

(SEQ ID NO: 3)
AUUUUAGUUCCUGGAUAAUUCAAGUUAGUGUAAAAC.

In some embodiments, the crRNA comprises a DR sequence comprising a sequence having at least about 80% identity to AUUUUAGUUCCUGGAUAAUUCA (SEQ ID NO: 4).

In some embodiments, the crRNA comprises a DR sequence comprising

(SEQ ID NO: 4)
AUUUUAGUUCCUGGAUAAUUCA.

In some embodiments, the crRNA sequence is fused to a target sequence.

In some embodiments, the crRNA sequence comprises a sequence of

(SEQ ID NO: 5)
NNNNNNNNNNNNNNNNNNNNAUUUUAGUUCCUGGAUAAUUCA.

In some embodiments, the tracrRNA comprises a sequence having at least about 80% identity to

(SEQ ID NO: 6)
UGAAUUAUUCAGACCAACUAAAACAAGGCUUUAUGCCGAAAUCAAGGAC
ACCUUCGGGUGUCCUUUUUU.

In some embodiments, the tracrRNA comprises a sequence of

(SEQ ID NO: 6)
UGAAUUAUUCAGACCAACUAAAACAAGGCUUUAUGCCGAAAUCAAGGAC
ACCUUCGGGUGUCCUUUUUU.

In some embodiments, the RNA guide comprises an sgRNA.

In some embodiments, the sgRNA comprises a scaffold comprising a sequence having at least about 80% identity to

(SEQ ID NO: 7)
AUUUUAGUUCCUGGAUAUAAUUAUUCAGACCAACUAAAACAAGGCUUUA
UGCCGAAAUCAAGGACACCUUCGGGUGUCCUUUUUUCUUUUU.

In some embodiments, the sgRNA comprises a scaffold comprising

(SEQ ID NO: 7)
AUUUUAGUUCCUGGAUAUAAUUAUUCAGACCAACUAAAACAAGGCUUUA
UGCCGAAAUCAAGGACACCUUCGGGUGUCCUUUUUUCUUUUU.

In some embodiments, the break in the target nucleic acid is a single-stranded or double-stranded break.

In some embodiments, the break in the target nucleic acid is a single-stranded break.

In some embodiments, the Cas9 protein is a nuclease that cleaves both strands of the target nucleic acid sequence, or is a nickase that cleaves one strand of the target nucleic acid sequence.

In some embodiments, the target nucleic acid is 5′ to a protospacer adjacent motif (PAM) sequence.

In some embodiments, the PAM has a sequence of 5′-NNGNG-3′.

In some embodiments, the Cas9 is operably linked to a promoter sequence for expression in a eukaryotic cell, and wherein the guide RNA is operably linked to a promoter sequence for expression in a eukaryotic cell.

In some embodiments, the eukaryotic cell is a human cell. In some embodiments, the eukaryotic cell is a plant cell.

In some embodiments, promoter sequence is a eukaryotic or viral promoter.

In one aspect, an engineered, non-naturally occurring CRISPR-Cas system is provided comprising: an RNA guide or a nucleic acid encoding the RNA guide, wherein the RNA guide comprises a direct repeat sequence and a spacer sequence capable of hybridizing to a target nucleic acid, and a codon-optimized CRISPR-associated (Cas) protein having at least 80% sequence identity to SEQ ID NO: 1, and wherein the Cas protein is capable of binding to the RNA guide and of causing a break in the target nucleic acid sequence complementary to the RNA guide.

In one aspect, an engineered, non-naturally occurring CRISPR-Cas system is provided comprising: an RNA guide or a nucleic acid encoding the RNA guide, wherein the RNA guide comprises a direct repeat sequence and a spacer sequence capable of hybridizing to a target nucleic acid; and a codon-optimized CRISPR-associated (Cas) protein having at least 80% sequence identity to SEQ ID NO: 1; wherein the Cas protein is fused to a deaminase, and wherein the Cas protein fusion is capable of binding to the RNA guide and of editing the target nucleic acid sequence complementary to the RNA guide.

In some embodiments, the Cas9 protein is an inactive Cas9 (dCas9).

In some embodiments, the RNA guide comprises a crRNA and a tracrRNA.

In some embodiments, the crRNA comprises a DR sequence comprising a sequence having at least about 801% identity to

(SEQ ID NO: 3)
AUUUUAGUUCCUGGAUAAUUCAAGUUAGUGUAAAAC.

In some embodiments, the crRNA comprises a DR sequence comprising

(SEQ ID NO: 3)
AUUUUAGUUCCUGGAUAAUUCAAGUUAGUGUAAAAC.

In some embodiments, the crRNA comprises a DR sequence comprising a sequence having at least about 80% identity to AUUUUAGUUCCUGGAUAAUUCA (SEQ ID NO: 4).

In some embodiments, the crRNA comprises a DR sequence comprising

(SEQ ID NO: 4)
AUUUUAGUUCCUGGAUAAUUCA.

In some embodiments, the crRNA sequence is fused to a target sequence.

In some embodiments, the crRNA sequence comprises a sequence of

(SEQ ID NO: 5)
NNNNNNNNNNNNNNNNNNNNAUUUUAGUUCCUGGAUAAUUCA.

In some embodiments, the tracrRNA comprises a sequence having at least about 80% identity to

(SEQ ID NO: 6)
UGAAUUAUUCAGACCAACUAAAACAAGGCUUUAUGCCGAAAUCAAGGAC
ACCUUCGGGUGUCCUUUUUU.

In some embodiments, the tracrRNA comprises a sequence of

(SEQ ID NO: 6)
UGAAUUAUUCAGACCAACUAAAACAAGGCUUUAUGCCGAAAUCAAGGAC
ACCUUCGGGUGUCCUUUUUU.

In some embodiments, the RNA guide comprises a sgRNA.

In some embodiments, the sgRNA comprises a scaffold comprising a sequence having at least about 80% identity to

(SEQ ID NO: 7)
AUUUUAGUUCCUGGAUAUAAUUAUUCAGACCAACUAAAACAAGGCUUUA
UGCCGAAAUCAAGGACACCUUCGGGUGUCCUUUUUUCUUUUU.

In some embodiments, the sgRNA comprises a scaffold comprising

(SEQ ID NO: 7)
AUUUUAGUUCCUGGAUAUAAUUAUUCAGACCAACUAAAACAAGGCUUUA
UGCCGAAAUCAAGGACACCUUCGGGUGUCCUUUUUUCUUUUU.

In some embodiments, the Cas protein is operably linked to a promoter sequence for expression in a eukaryotic cell, and wherein the guide RNA is operably linked to a promoter sequence for expression in a eukaryotic cell.

In some embodiments, the eukaryotic cell is a human cell. In some embodiments, the eukaryotic cell is a plant cell.

In some embodiments, the promoter sequence is a eukaryotic promoter sequence.

In some embodiments, a nucleic acid encoding the system as described herein is provided.

In some embodiments, a vector comprising the system as described herein is provided.

In some embodiments, the vector is a plasmid vector or a viral vector.

In some embodiments the viral vector is an adeno associated virus (AAV) vector or a lentiviral vector.

In some embodiments the viral vector is an AAV vector.

In some embodiments more than one AAV vector is used for packaging the system of any aspect delineated herein.

In one aspect, a method of treating a disorder or a disease in a subject in need thereof is provided, the method comprising administering to the subject a system as described herein, wherein the guide RNA is complementary to at least 10 nucleotides of a target nucleic acid associated with the condition or disease; wherein the Cas protein associates with the guide RNA; wherein the guide RNA binds to the target nucleic acid; wherein the Cas protein causes a break in the target nucleic acid, optionally wherein the Cas9 is an inactive Cas9 (dCas9) fused to a deaminase and results in one or more base edits in the target nucleic acid, thereby treating the disorder or disease.

In some embodiments, the guide RNA is complementary to about 18-24 nucleotides.

In some embodiments, the guide RNA is complementary to 20 nucleotides.

In one aspect, a base editor is provided herein comprising a non-naturally occurring Cas9 fusion protein comprising a Cas9 protein having at least 80% identity to SEQ ID NO: 1.

In some embodiments, the base editor comprises an adenosine deaminase domain or a cytidine deaminase domain. In some embodiments, the base editor is a multi-effector base editor comprising two or more nucleobase editing domains (e.g., comprising an adenosine deaminase domain and a cytidine deaminase domain).

In one aspect, a method of editing a nucleobase of a polynucleotide is provided herein, the method comprising contacting the polynucleotide with a base editor in complex with one or more guide RNAs, wherein the base editor comprises an adenosine deaminase domain, and wherein the one or more guide RNAs target the base editor to effect an A⋅T to G⋅C alteration in the polynucleotide.

In one aspect, a method of editing a nucleobase of a polynucleotide is provided herein, the method comprising contacting the polynucleotide with a base editor in complex with one or more guide RNAs, wherein the base editor comprises a cytidine deaminase domain, and wherein the one or more guide RNAs target the base editor to effect an C⋅G to T⋅A alteration in the polynucleotide.

In some embodiments, the editing results in less than 50% indel formation in the target polynucleotide sequence.

In some embodiments, the editing generates a point mutation.

### Definitions

In order for the present invention to be more readily understood, certain terms are first defined below. Additional definitions for the following terms and other terms are set forth throughout the specification.

A or An: The articles “a” and “an” are used herein to refer to one or to more than one (i.e., to at least one) of the grammatical object of the article. By way of example, “an element” means one element or more than one element.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%/0, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: Two events or entities are “associated” with one another, as that term is used herein, if the presence, level and/or form of one is correlated with that of the other. For example, a particular entity (e.g., polypeptide) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Base Editor: By “base editor (BE),” or “nucleobase editor (NBE)” is meant an agent that binds a polynucleotide and has nucleobase modifying activity. In various embodiments, the base editor comprises a nucleobase modifying polypeptide (e.g., a deaminase) and a polynucleotide programmable nucleotide binding domain in conjunction with a guide polynucleotide (e.g., guide RNA). In various embodiments, the agent is a biomolecular complex comprising a protein domain having base editing activity, i.e., a domain capable of modifying a base (e.g., A, T, C, G, or U) within a nucleic acid molecule (e.g., DNA). In some embodiments, the polynucleotide programmable DNA binding domain is fused or linked to a deaminase domain. In one embodiment, the agent is a fusion protein comprising one or more domains having base editing activity. In another embodiment, the protein domains having base editing activity are linked to the guide RNA (e.g., via an RNA binding motif on the guide RNA and an RNA binding domain fused to the deaminase). In some embodiments, the domains having base editing activity are capable of deaminating a base within a nucleic acid molecule. In some embodiments, the base editor is capable of deaminating one or more bases within a DNA molecule. In some embodiments, the base editor is capable of deaminating a cytosine (C) or an adenosine (A) within DNA. In some embodiments, the base editor is capable of deaminating a cytosine (C) and an adenosine (A) within DNA. In some embodiments, the base editor is a cytidine base editor (CBE). In some embodiments, the base editor is an adenosine base editor (ABE). In some embodiments, the base editor is an adenosine base editor (ABE) and a cytidine base editor (CBE). In some embodiments, the base editor is a nuclease-inactive Cas9 (dCas9) fused to an adenosine deaminase. In some embodiments, the base editor is fused to an inhibitor of base excision repair, for example, a UGI domain, or a dISN domain. In some embodiments, the fusion protein comprises a Cas9 nickase fused to a deaminase and an inhibitor of base excision repair, such as a UGI or dISN domain. In other embodiments the base editor is an abasic base editor. Details of base editors are described in International PCT Application Nos. PCT/2017/045381 (WO2018/027078) and PCT/US2016/058344 (WO2017/070632), each of which is incorporated herein by reference for its entirety. Also see Komor, A. C., et al., “Programmable editing of a target base in genomic DNA without double-stranded DNA cleavage” Nature 533, 420-424 (2016); Gaudelli, N. M., et al., “Programmable base editing of A⋅T to G⋅C in genomic DNA without DNA cleavage” Nature 551, 464-471 (2017); Komor, A. C., et al., “Improved base excision repair inhibition and bacteriophage Mu Gam protein yields C:G-to-T:A base editors with higher efficiency and product purity” Science Advances 3:eaao4774 (2017), and Rees, H. A., et al., “Base editing: precision chemistry on the genome and transcriptome of living cells.” Nat Rev Genet. 2018 December; 19(12):770-788. doi: 10.1038/s41576-018-0059-1, the entire contents of which are hereby incorporated by reference.

Base Editing Activity: By “base editing activity” is meant acting to chemically alter a base within a polynucleotide. In one embodiment, a first base is converted to a second base. In one embodiment, the base editing activity is cytidine deaminase activity, e.g., converting target C⋅G to T⋅A. In another embodiment, the base editing activity is adenosine or adenine deaminase activity, e.g., converting A⋅T to G⋅C. In another embodiment, the base editing activity is cytidine deaminase activity. e.g., converting target C⋅G to T⋅A and adenosine or adenine deaminase activity, e.g., converting A⋅T to G⋅C.

Base Editor System: The term “base editor system” refers to a system for editing a nucleobase of a target nucleotide sequence. In various embodiments, the base editor (BE) system comprises (1) a polynucleotide programmable nucleotide binding domain (e.g., Cas9), a deaminase domain and a cytidine deaminase domain for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domains selected from an adenosine deaminase or a cytidine deaminase, and a domain having nucleic acid sequence specific binding activity. In some embodiments, the base editor system comprises (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain. In some embodiments, the polynucleotide programmable nucleotide binding domain is a polynucleotide programmable DNA binding domain. In some embodiments, the base editor is a cytidine base editor (CBE). In some embodiments, the base editor is an adenine or adenosine base editor (ABE). In some embodiments, the base editor is an adenine or adenosine base editor (ABE) or a cytidine base editor (CBE).

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active. In particular embodiments, where a peptide is biologically active, a portion of that peptide that shares at least one biological activity of the peptide is typically referred to as a “biologically active” portion.

Cleavage: As used herein, cleavage refers to a break in a target nucleic acid created by a nuclease of a CRISPR system described herein. In some embodiments, the cleavage event is a double-stranded DNA break. In some embodiments, the cleavage event is a single-stranded DNA break. In some embodiments, the cleavage event is a single-stranded RNA break. In some embodiments, the cleavage event is a double-stranded RNA break.

Complementary: As used herein, complementary refers to a nucleic acid strand that forms Watson-Crick base pairing, such that A base pairs with T, and C base pairs with G, or non-traditional base pairing with bases on a second nucleic acid strand. In other words, it refers to nucleic acids that hybridize with each other under appropriate conditions.

Clustered Interspaced Short Palindromic Repeat (CRISPR)-associated (Cas) system: As used herein, CRISPR-Cas9 system refers to nucleic acids and/or proteins involved in the expression of, or directing the activity of, CRISPR-effectors, including sequences encoding CRISPR effectors, RNA guides, and other sequences and transcripts from a CRISPR locus. In some embodiments, the CRISPR system is an engineered, non-naturally occurring CRISPR system. In some embodiments, the components of a CRISPR system may include a nucleic acid(s) (e.g., a vector) encoding one or more components of the system, a component(s) in protein form, or a combination thereof.

CRISPR Array: The term “CRISPR array”, as used herein, refers to the nucleic acid (e.g., DNA) segment that includes CRISPR repeats and spacers, starting with the first nucleotide of the first CRISPR repeat and ending with the last nucleotide of the last (terminal) CRISPR repeat. Typically, each spacer in a CRISPR array is located between two repeats. The terms “CRISPR repeat” or “CRISPR direct repeat,” or “direct repeat.” as used herein, refer to multiple short direct repeating sequences, which show very little or no sequence variation within a CRISPR array.

CRISPR-associated protein (Cas): The term “CRISPR-associated protein,” “CRISPR effector,” “effector,” or “CRISPR enzyme” as used herein refers to a protein that carries out an enzymatic activity or that binds to a target site on a nucleic acid specified by a RNA guide. In different embodiments, a CRISPR effector has endonuclease activity, nickase activity, exonuclease activity, transposase activity, and/or excision activity.

crRNA: The term “CRISPR RNA” or “crRNA,” as used herein, refers to a RNA molecule including a guide sequence used by a CRISPR effector to target a specific nucleic acid sequence. Typically, crRNAs contains a sequence that mediates target recognition and a sequence that forms a duplex with a tracrRNA. In some embodiments, the crRNA:tracrRNA duplex binds to a CRISPR effector.

Ex Vivo: As used herein, the term “ex vivo” refers to events that occur in cells or tissues, grown outside rather than within a multi-cellular organism.

Functional equivalent or analog: As used herein, the term “functional equivalent” or “functional analog” denotes, in the context of a functional derivative of an amino acid sequence, a molecule that retains a biological activity (either function or structural) that is substantially similar to that of the original sequence. A functional derivative or equivalent may be a natural derivative or is prepared synthetically. Exemplary functional derivatives include amino acid sequences having substitutions, deletions, or additions of one or more amino acids, provided that the biological activity of the protein is conserved. The substituting amino acid desirably has chemico-physical properties which are similar to that of the substituted amino acid. Desirable similar chemico-physical properties include, similarities in charge, bulkiness, hydrophobicity, hydrophilicity, and the like.

Half-Life: As used herein, the term “half-life” is the time required for a quantity such as protein concentration or activity to fall to half of its value as measured at the beginning of a time period.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

Inhibition: As used herein, the terms “inhibition,” “inhibit” and “inhibiting” refer to processes or methods of decreasing or reducing activity and/or expression of a protein or a gene of interest. Typically, inhibiting a protein or a gene refers to reducing expression or a relevant activity of the protein or gene by at least 10% or more, for example, 20%, 30%, 40%, or 50%, 60%, 70%, 80%, 90% or more, or a decrease in expression or the relevant activity of greater than 1-fold, 2-fold, 3-fold, 4-fold, 5-fold, 10-fold, 50-fold, 100-fold or more as measured by one or more methods described herein or recognized in the art.

Hybridization: As used herein, the term “hybridization” refers to a reaction in which two or more nucleic acids bind with each other via hydrogen bonding by Watson-Crick pairing, Hoogstein binding or other sequence-specific binding between the bases of the two nucleic acids. A sequence capable of hybridizing with another sequence is termed the “complement” of the sequence, and is said to be “complementary” or show “complementarity”.

Indel: As used herein, the term “indel” refers to insertion or deletion of bases in a nucleic acid sequence. It commonly results in mutations and is a common form of genetic variation.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Mutation: As used herein, the term “mutation” has the ordinary meaning in the art, and includes, for example, point mutations, substitutions, insertions, deletions, inversions, and deletions.

Oligonucleotide: As used herein, the term “oligonucleotide” generally refers to polynucleotides of between about 5 and about 100 nucleotides of single- or double-stranded DNA. Oligonucleotides are also known as “oligomers” or “oligos” and may be isolated from genes, or chemically synthesized.

PAM: The term “PAM” or “Protospacer Adjacent Motif” refers to a short nucleic acid sequence (usually 2-6 base pairs in length) that follows the nucleic acid region targeted for cleavage by the CRISPR system, such as CRISPR-Cas9. The PAM is required for a Cas nuclease to cut and is generally found 3-4 nucleotides downstream from the cut site.

Polypeptide: The term “polypeptide” as used herein refers to a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified. As used herein, the terms “polypeptide” and “peptide” are used inter-changeably.

Prevent: As used herein, the term “prevent” or “prevention”, when used in connection with the occurrence of a disease, disorder, and/or condition, refers to reducing the risk of developing the disease, disorder and/or condition.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

Reference: A “reference” entity, system, amount, set of conditions, etc., is one against which a test entity, system, amount, set of conditions, etc. is compared as described herein. For example, in some embodiments, a “reference” antibody is a control antibody that is not engineered as described herein.

RNA guide: The term RNA guide refers to an RNA molecule that facilitates the targeting of a protein described herein to a target nucleic acid. Exemplary “RNA guides” or “guide RNAs” include, but are not limited to, crRNAs or crRNAs in combination with cognate tracrRNAs. The latter may be independent RNAs or fused as a single RNA using a linker (sgRNAs). In some embodiments, the RNA guide is engineered to include a chemical or biochemical modification, in some embodiments, an RNA guide may include one or more nucleotides.

Subject: The term “subject”, as used herein, means any subject for whom diagnosis, prognosis, or therapy is desired. For example, a subject can be a mammal, e.g., a human or non-human primate (such as an ape, monkey, orangutan, or chimpanzee), a dog, cat, guinea pig, rabbit, rat, mouse, horse, cattle, or cow.

sgRNA: The term “sgRNA” or “single guide RNA” refers to a single guide RNA containing (i) a guide sequence (crRNA sequence) and (ii) a Cas9 nuclease-recruiting sequence (tracrRNA).

Substantial identity: The phrase “substantial identity” is used herein to refer to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3); 403-410, 1990; Altschul, et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Target Nucleic Acid: The term “target nucleic acid” as used herein refers to nucleotides of any length (oligonucleotides or polynucleotides) to which the CRISPR-Cas9 system binds, either deoxyribonucleotides, ribonucleotides, or analogs thereof. Target nucleic acids may have three-dimensional structure, may including coding or non-coding regions, may include exons, introns, mRNA, tRNA, rRNA, siRNA, shRNA, miRNA, ribozymes, cDNA, plasmids, vectors, exogenous sequences, endogenous sequences. A target nucleic acid can comprise modified nucleotides, include methylated nucleotides, or nucleotide analogs. A target nucleic acid may be interspersed with non-nucleic acid components. A target nucleic acid is not limited to, single-, double-, or multi-stranded DNA or RNA, genomic DNA. cDNA, DNA-RNA hybrids, or a polymer comprising purine and pyrimidine bases or other natural, chemically or biochemically modified, non-natural, or derivatized nucleotide bases.

Therapeutically effective amount: As used herein, the term “therapeutically effective amount” refers to an amount of a therapeutic molecule (e.g., an engineered antibody described herein) which confers a therapeutic effect on a treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic molecule or composition effective to treat, ameliorate, or prevent a particular disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount can be administered in a dosing regimen that may comprise multiple unit doses. For any particular therapeutic molecule, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular subject may depend upon a variety of factors including the disorder being treated and the severity of the disorder; the activity of the specific pharmaceutical agent employed, the specific composition employed; the age, body weight, general health, sex and diet of the subject; the time of administration, route of administration, and/or rate of excretion or metabolism of the specific therapeutic molecule employed; the duration of the treatment; and like factors as is well known in the medical arts.

tracrRNA: The term “tracrRNA” or “trans-activating crRNA” as used herein refers to an RNA including a sequence that forms a structure required for a CRISPR-associated protein to bind to a specified target nucleic acid.

Treatment: As used herein, the term “treatment” (also “treat” or “treating”) refers to any administration of a therapeutic molecule (e.g., a CRISPR-Cas therapeutic protein or system described herein) that partially or completely alleviates, ameliorates, relieves, inhibits, delays onset of, reduces severity of and/or reduces incidence of one or more symptoms or features of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition.

### BRIEF DESCRIPTION OF THE DRAWING

Drawings are for illustration purposes only; not for limitation.

FIG. 1 is a graph that shows a consensus PAM motif for human codon-optimized Lachnospira UBA3212 Cas9.

FIG. 2A is a schematic that shows predicted RNA folding structure of crRNA, and tracrRNA for human codon-optimized Lachnospira UBA3212 Cas9 using Geneious software (geneious.com). FIG. 2B is a schematic that shows predicted RNA folding structure of sgRNA for human codon-optimized Lachnospira UBA3212 Cas9 using Geneious software. FIG. 2C-2M show the predicted RNA folding structure of sgRNAs 1-11, respectively.

FIG. 3 is a gel that shows exemplary results of in vitro cleavage activity measurements of human codon-optimized Lachnospira UBA3212 Cas9 directed to an FnPSP1 target site.

FIG. 4 is a graph that shows exemplary results of ex vivo cleavage activity of human codon-optimized Lachnospira UBA3212 Cas9 in HEK293T cells. The y-axis of the graph shows indel frequency obtained using various guide RNAs that targeted A-rich genomic test sites adjacent to a sequence corresponding to the PAM consensus motif (see FIG. 1).

FIG. 5A is a graph that shows results of adenine to guanine base (A-to-G) conversion percentage achieved with a base editor comprising a TadA8 adenine deaminase fused to the N-terminus of a Lachnospira UBA3212 Cas9 D8A mutant. A-to-G conversion percentage (y-axis) plotted for various guide RNAs targeting A-rich genomic test sites (x-axis: Table 12) adjacent to a sequence corresponding to the PAM consensus motif (see FIG. 1).

FIG. 5B is a graph that shows the indel frequencies at the sites tested for A-to-G conversion in FIG. 5A. Indel frequency (y-axis) is plotted for the genomic test sites (x-axis) presented in FIG. 5A.

FIG. 6A is a graph that shows results of A-to-G base conversion percentage achieved with a base editor comprising a TadA8 adenine deaminase fused to the C-terminus of a Lachnospira UBA3212 Cas9 D8A mutant. The y-axis of the graph shows the A to G conversion percentage (y-axis) plotted for various guide RNAs targeting A-rich genomic test sites (x-axis; Table 12) adjacent to a sequence corresponding to the PAM consensus motif (see FIG. 1).

FIG. 6B is a graph that shows the indel frequencies at the sites tested for A-to-G conversion in FIG. 6A. Indel frequency (y-axis) is plotted for the genomic test sites (x-axis) presented in FIG. 6A.

FIG. 7A is a graph examining the base editing window of a base editor comprising TadA8 adenine deaminase fused to the N-terminus of a Lachnospira UBA3212 Cas9 D8A mutant. The graph shows A-to-G conversion (y-axis) obtained at each adenine residue (x-axis) specified by the sequence shown for guide RNA 10.

FIG. 7B is a graph that examines the base editing window of a base editor comprising TadA8 adenine deaminase fused to the N-terminus of a Lachnospira UBA3212 Cas9 D8A mutant. The graph shows A-to-G conversion (y-axis) obtained at each adenine residue (x-axis) specified by the sequence shown for guide RNA 12.

FIG. 8A-8D show data for LubCas9 nuclease activity using various sgRNAs of different designs and guide lengths. FIG. 8A shows indel frequency using LubCas9 nuclease with different sgRNAs and guide lengths for targeting EMX site 9. FIG. 8B. shows indel frequency using LubCas9 nuclease with different sgRNAs and guide lengths for targeting VEGFA site 22. FIG. 8C shows indel frequency using LubCas9 nuclease with different sgRNAs and guide lengths for targeting VEGFA site 23. FIG. 8D shows data using LubCas9 nuclease using targeting EMX1 site 9, VEGFA site 22, VEGFA site 23, and Hek4 site 708.

FIG. 9A-9C show LubCas9 nuclease activity when fused to either an adenine base editor (ABE) or cytosine base editor (CBE). FIG. 9A shows nuclease activity using ABE-dLubCas9 using different sgRNA designs and guide lengths for targeting VEGFA site 22 or 23. FIG. 9B shows ABE-d-LubCas9 nuclease activity using various sgRNAs and 21 nucleotide guides. FIG. 9C shows CBE-dlubCas9 nuclease activity using various sgRNAs and 21 nucleotide guides.

### DETAILED DESCRIPTION

Clustered regularly interspaced short palindromic repeats (CRISPR) was first discovered as an adaptive immune system in bacteria and archaea, and then engineered to generate targeted DNA breaks in living cells and organisms. During the cellular DNA repair process, various DNA changes can be introduced. The diverse and expanding CRISPR toolbox allows programmable genome editing, epigenome editing and transcriptome regulation.

CRISPR-Cas systems comprise three main types (I, II, and III) based on their Cas gene organization, and the sequence and structure of component proteins. Each of the three CRISPR systems is characterized by a unique Cas gene: Cas3, a target-degrading nuclease/helicase in Type I; Cas9, an RNA-binding and target-degrading nuclease in type II; Cas10, a large protein for multiple functions in type III. The three CRISPR types also differ in their associated effector complexes. Type I Cas systems associate with Cascade effector complexes, type II effector complexes consist of a single Cas9 and one or more RNA molecules, and type III interference complexes are further divided into type III-A (Csm complex targeting DNA) and type III-B (Cmr complex targeting RNA). Cas proteins are important components of effector complexes in all CRISPR-Cas systems.

Current genome editing technologies have focused on Class II CRISPR-Cas systems, which contain single-protein effector nucleases for DNA cleavage, specifically, Cas9, a dual-RNA-guided nuclease which requires both CRISPR RNA (crRNA) and tracrRNA and contains both HNH and RuvC nuclease domains, and Cas12a, a single-RNA-guided nuclease which only requires crRNA and contains a single RuvC domain.

Various aspects of the invention are described in detail in the following sections. The use of sections is not meant to limit the invention. Each section can apply to any aspect of the invention. In this application, the use of “or” means “and/or” unless stated otherwise.

### Engineered, Non-Naturally Occurring Cas9 Protein

Described herein is an engineered, non-naturally occurring Cas9 protein modified from Lachnospira UBA3212 bacteria Cas9.

In some embodiments, the engineered non-naturally occurring Cas9 protein described herein comprises an amino acid sequence at least 60% (e.g., 60%, 65%, 70%, 75%, 80%, 81%, 82%, 83%, 84%, 85%, 86%, 87%, 88%, 89%., 90%, 91%, 92%, 93%, 94%, 95%, 96%, 97%, 98%, 99% or more) identical to SEQ ID NO: 1. In some embodiments, the Cas9 protein has is 80% identical to SEQ ID NO: 1. In some embodiments, the amino acid sequence of the Cas9 protein is identical to SEQ ID NO: 1. Exemplary Cas9 amino acid sequences are provided in Table 1 below.

TABLE 1
Exemplary Cas9 Amino Acid Sequences

Wild Type Lachnospira UBA3212 Cas9
MSVNVGLDIGIASVGVAVVDSESGEILEAVSDLFESAEANQNVDRRGFRQSRRLKRRQYNR
IHDFMKLWEEFGFVKPENINLNTVGLRVKSLTEQVTLDELYVILLSELKHRGISYLEDSEE
VDGGSEYKEGLRINQRELQSKYPCEIQLERLKIYGRYRGNFTVEIDGEKVGLSNVFTTGAY
RKEIQQLLSIQKTYQSKLTDDFINKYLEIFDRKRQYYVGPGNEKSRTDYGRYTTKKDAEGN
YITDENIFEKLIGKCSIYPEEMRAAGASYTAQEFNLLNDLNNLTIGGRKIEEEEKRAIIET
IKSSKVVNVEKIICKVTGEDAETITGARIDKDDKRIYHSFECYRKLKKALETIEVKIEEYS
REELDELARILTLNTEREGILGELEKSELDLGEEVIDCVIDERRKNGPLFSKWQSFSLRLM
NDIIPDMYEQPKEQMTLLTEMGLMKSKKEIFKGMKYIPENVMRDDIYNPVVVRSVRIAVRA
LNAVIKKYGEIDKVVIEMPRDRNTEEQKKRIDAENKRNREELPGIEKRILEEYGIKITSAH
YRNHKQLGLKLKLWNEQGGICPYSGKTIDLERLLQNAGDYEVDHIIPLSISLDDSRNNKVL
VYASENQKKGNQTPYAYLSSVQREWGWEQYRHYVLSDLKKKKISSKKIENYLFMKDISKID
VVKGFIQRNLNDTRYASKVVLNTLESFFKANEKETKVSVIRGSFTSLMRKNLKLDKSREES
YAHHAVDALLIAYSKMGYDSYHKLQGEFIDFETGEILDSRMWETNLEPDILKGYLYGRKWS
EIRENIKIAESRVKYWHMTNKKCNRSLCNQTLYGTRTYDGKIYQIKKIKDIRTPEGLKTFK
DLVDKNKGDHLLMARNDPKTYEQILQIYRDYSDAKNPFLQYEMETGDCIRKYSKKHNGSRI
VSLKYHDGEVNSCIDVSHKYGFEKGSQKVVLMSLNPYRMDVYKNCNDGKYYLIGLKQSDIK
CEGRHYVIDEEKYAKVLVNEKMIQPGQSRKDLPDLGYEFVMSFYKNEIIQYEKDGKFYKER
FLSRTKPASRNYIETKPVDKPNFEKRHQIGLAKTTFIRKIRTDILGNEYNCDREKESSIC
(SEQ ID NO: 1)
Lachnospira UBA3212 Cas9 with Nuclear Localization Signal (NLS)
and Linker
MPKKKRKVGSVNVGLDIGIASVGVAVVDSESGEILEAVSDLFESAEANQNVDRRGFRQSRR
LKRRQYNRIHDFMKLWEEFGFVKPENINLNTVGLRVKSLTEQVTLDELYVILLSELKHRGI
SYLEDSEEVDGGSEYKEGLRINQRELQSKYPCEIQLERLKIYGRYRGNFTVEIDGEKVGLS
NVFTTGAYRKEIQQLLSIQKTYQSKLTDDFINKYLEIFDRKRQYYVGPGNEKSRTDYGRYT
TKKDAEGNYITDENIFEKLIGKCSTYPEEMRAAGASYTAQEENLLNDLNNLTIGGRKIEEE
EKRAIIETIKSSKVVNVEKIICKVTGEDAETITGARIDKDDKRIYHSFECYRKLKKALETI
EVKIEEYSREELDELARILTLNTEREGILGELEKSFLDLGEEVIDCVIDERRKNGPLESKW
QSFSLRLMNDIIPDMYEQPKEQMTLLTEMGLMKSKKEIFKGMKYIPENVMRDDIYNPVVVR
SVRIAVRALNAVIKKYGEIDKVVIEMPRDRNTEEQKKRIDAENKRNREELPGIEKRILEEY
GIKITSAHYRNHKQLGLKLKLWNEQGGICPYSGKTIDLERLLQNAGDYEVDHIIPLSISLD
DSRNNKVLVYASENQKKGNQTPYAYLSSVQREWGWEQYRHYVLSDLKKKKISSKKIENYLE
MKDISKIDVVKGFIQRNLNDTRYASKVVLNTLESFFKANEKETKVSVIRGSFTSLMRKNLK
LDKSREESYAHHAVDALLIAYSKMGYDSYHKLQGEFIDFETGEILDSRMWETNLEPDILKG
YLYGRKWSEIRENIKIAESRVKYWHMTNKKCNRSLCNQTLYGTRTYDGKIYQIKKIKDIRT
PEGLKTFKDLVDKNKGDHLLMARNDPKTYEQILQIYRDYSDAKNPFLQYEMETGDCIRKYS
KKHNGSRIVSLKYHDGEVNSCIDVSHKYGFEKGSQKVVLMSLNPYRMDVYKNCNDGKYYLI
GLKQSDIKCEGRHYVIDEEKYAKVLVNEKMIQPGQSRKDLPDLGYEFVMSFYKNEIIQYEK
DGKFYKERFLSRTKPASRNYIETKPVDKPNFEKRHQIGLAKTTFIRKIRTDILGNEYNCDR
EKFSSICKRPAATKKAGQAKKKKGSYPYDVPDYAYPYDVPDYAYPYDVPDYA

NLS (bold), can be substituted with different NLSs
Linker (underlined), can be removed or extended
3×HA tag (italics), can be substituted with different tags

In some embodiments, the Cas9 protein comprises one or more mutations in reference to SEQ ID NO: 1. For example, the amino acid sequence of the Cas9 protein comprises at least one, at least two, at least three, at least four, at least five, at least six, at least seven, at least eight, at least nine, at least 10 mutations in SEQ ID NO: 1. Various mutations are known in the art, and include for example, amino acid substitutions.

In some embodiments, two or more catalytic domains of Cas9 (RuvC1, RuvCII, RuvCIII) are mutated to produce an inactive, or “dead” Cas9 (dCas9) that lacks nucleic acid cleavage activity. In some embodiments, the one or more mutations are in the PAM Interacting, HNH, and or the RuvC domains. In some embodiments. Cas9 is mutated to reduce DNA cleavage activity to less than about 25%, 15%, 10%, 5%, 1%, 0.1%, 0.01% or lower with respect to its non-mutated form.

In some embodiments, the mutation is an aspartic acid-to-alanine substitution (D8A) in the RuvC domain of Cas9 (e.g., corresponding to D10A in SpCas9). In some embodiments, the mutation is a histidine-to-alanine substitution (H593A) in the HNH domain of Cas9 (e.g., corresponding to H840A in SpCas9). In some embodiments, the Cas9 protein comprises one or more mutations at residues D8, H593, and/or N616. In some embodiments, the Cas9 protein comprises a D8A, H593A and/or N616A mutation of the amino acid sequence provided in SEQ ID NO: 1. In some embodiments, the Cas9 protein comprises a D8N, H593N and/or N616N mutation of the amino acid sequence provided in SEQ ID NO: 1, where N is any amino acid. Such one or more mutations, for example, converts Cas9 to an inactive, or “dead” version of Cas9 (dCas9). Accordingly, in some embodiments, the Cas9 protein comprises one or more mutations that inhibits the ability of Cas9 to cleave both strands of a DNA duplex.

In some embodiments, when coexpressed with a guide RNA, dead Cas9 generates a DNA recognition complex that can specifically interfere with transcriptional elongation, RNA polymerase binding, or transcription factor binding. In some embodiments, dead Cas9 is used to specifically target effector proteins of various functions to specific nucleic acid target sites.

In some embodiments, the engineered non-naturally occurring Cas9 is codon-optimized for human cells. The engineered, non-naturally occurring Cas9 is encoded by a nucleic acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99% or more) identical to SEQ ID NO. 2. In some embodiments, the Cas9 is encoded by a nucleic acid sequence that is identical to SEQ ID NO: 2. An exemplary Cas9 nucleotide sequence with Nuclear Localization Signal (NLS) and a linker is provided in Table 2 below.

TABLE 2
Exemplary Cas9 Nucleotide Sequence with NLS and Linker

Nucleotide Sequence of Lachnospira UBA3212 Cas9 with Nuclear
Localization Signal (NLS) and Linker
ATGCCCAAGAAGAAGCGGAAGGTTGGTTCTGTCAACGTGGGGCTGGATATTGGCATCGCAT
CAGTGGGAGTCGCCGTGGTGGATAGTGAAAGTGGAGAGATTCTGGAGGCTGTGTCCGACCT
GTTCGAGTCTGCCGAGGCCAACCAGAATGTGGATCGGAGAGGCTTTAGACAGAGCAGGCGC
CTGAAGCGGAGACAGTATAATAGGATCCACGACTTCATGAAGCTGTGGGAGGAGTTCGGCT
TTGTGAAGCCCGAGAACATCAATCTGAACACCGTGGGACTGAGGGTGAAGAGCCTGACCGA
GCAGGTGACACTGGATGAGCTGTACGTGATCCTGCTGTCCGAGCTGAAGCACCGGGGCATC
AGCTATCTGGAGGACTCCGAGGAGGTGGATGGAGGATCCGAGTACAAGGAGGGACTGCGGA
TCAACCAGAGAGAGCTGCAGTCTAAGTATCCTTGCGAGATCCAGCTGGAGAGACTGAAGAT
CTACGGCCGGTATAGAGGCAATTTCACCGTGGAGATCGACGGCGAGAAAGTGGGCCTGAGC
AACGTGTTTACCACAGGCGCCTACAGGAAGGAGATCCAGCAGCTGCTGTCTATCCAGAAGA
CCTATCAGAGCAAGCTGACAGACGATTTCATCAATAAGTACCTGGAGATCTTTGACAGGAA
GCGCCAGTACTATGTGGGCCCAGGCAACGAGAAGTCCCGGACCGATTACGGCAGATATACC
ACAAAGAAGGACGCCGAGGGCAATTACATCACAGATGAGAACATCTTCGAGAAGCTGATCG
GCAAGTGTAGCATCTATCCAGAGGAGATGAGGGCAGCAGGAGCATCCTACACCGCCCAGGA
GTTTAATCTGCTGAACGACCTGAACAATCTGACAATCGGCGGCCGGAAGATCGAGGAGGAG
GAGAAGAGAGCCATCATCGAGACCATCAAGAGCTCCAAGGTGGTGAATGTGGAGAAGATCA
TCTGCAAGGTGACAGGAGAGGACGCAGAGACCATCACAGGAGCAAGGATCGATAAGGACGA
TAAGCGCATCTATCACTCCTTCGAGTGTTACAGAAAGCTGAAGAAGGCCCTGGAGACCATC
GAGGTGAAGATCGAGGAGTACTCTAGGGAGGAGCTGGACGAGCTGGCAAGGATCCTGACCC
TGAACACAGAGAGGGAGGGAATCCTGGGAGAGCTGGAGAAGTCTTTCCTGGATCTGGGCGA
GGAAGTGATCGACTGCGTGATCGACTTCCGGCGCAAGAATGGCCCTCTGTTCAGCAAGTGG
CAGAGCTTTTCCCTGAGGCTGATGAACGACATCATCCCAGATATGTATGAGCAGCCCAAGG
AGCAGATGACCCTGCTGACAGAGATGGGCCTGATGAAGAGCAAGAAGGAGATCTTTAAGGG
CATGAAGTATATCCCCGAGAATGTGATGAGAGACGATATCTACAACCCTGTGGTGGTGCGG
TCCGTGAGAATCGCCGTGAGGGCCCTGAATGCCGTGATCAAGAAGTACGGCGAGATCGACA
AGGTGGTCATCGAGATGCCTCGGGATAGAAACACCGAGGAGCAGAAGAAGCGGATCGACGC
CGAGAATAAGAGGAACCGCGAGGAGCTGCCAGGCATCGAGAAGAGAATCCTGGAGGAGTAT
GGCATCAAGATCACCTCCGCCCACTACAGGAATCACAAGCAGOTGGGCCTGAAGCTGAAGC
TGTGGAACGAGCAGGGCGGCATCTGTCCCTATTCTGGCAAGACAATCGATCTGGAGAGACT
GCTGCAGAACGCCGGCGACTACGAGGTGGATCACATCATCCCTCTGTCTATCAGCCTGGAC
GATTCTAGGAACAATAAGGTGCTGGTGTACGCCAGCGAGAATCAGAAGAAGGGCAACCAGA
CCCCCTACGCCTATCTGTCTAGCGTGCAGAGAGAGTGGGGCTGGGAGCAGTACAGGCACTA
TGTGCTGAGCGACCTGAAGAAGAAGAAGATCTCCTCTAAGAAGATCGAGAATTATCTGTTC
ATGAAGGACATCTCCAAGATCGATGTGGTGAAGGGCTTTATCCAGAGGAATCTGAACGATA
CCCGCTACGCCAGCAAGGTGGTGCTGAATACACTGGAGTCCTTCTTTAAGGCCAACGAGAA
GGAGACCAAGGTGAGCGTGATCCGCGGCTCCTTCACATCTCTGATGCGGAAGAACCTGAAG
CTGGACAAGAGCAGGGAGGAGTCCTATGCACACCACGCAGTGGACGCACTGCTGATCGCCT
ACTCCAAGATGGGCTACGATTCTTATCACAAGCTGCAGGGCGAGTTCATCGACTTTGAGAC
CGGCGAGATCCTGGATAGCCGCATGTGGGAGACAAATCTGGAGCCTGATATCCTGAAGGGC
TACCTGTATGGCCGGAAGTGGTCCGAGATCAGAGAGAACATCAAGATCGCCGAGTCTCGGG
TGAAGTACTGGCACATGACCAATAAGAAGTGCAACCGGAGCCTGTGCAACCAGACACTGTA
CGGCACCCGGACATATGACGGCAAGATCTACCAGATCAAGAAGATCAAGGATATCCGCACC
CCAGAGGGCCTGAAGACATTCAAGGACCTGGTGGATAAGAATAAGGGCGACCACCTGCTGA
TGGCCCGCAACGATCCAAAGACCTACGAGCAGATCCTGCAGATCTACCGGGACTATTCTGA
TGCCAAGAATCCCTTTCTGCAGTATGAGATGGAGACAGGCGACTGCATCAGAAAGTACAGC
AAGAAGCACAATGGCTCTAGGATCGTGAGCCTGAAGTATCACGACGGCGAGGTGAACTCTT
GTATCGATGTGAGCCACAAGTACGGCTTCGAGAAGGGCTCCCAGAAGGTGGTGCTGATGTC
TCTGAACCCATATCGGATGGACGTGTACAAGAATTGCAACGATGGCAAGTACTATCTGATC
GGCCTGAAGCAGTCCGACATCAAGTGTGAGGGCCGCCACTATGTGATCGATGAGGAGAAGT
ACGCCAAGGTGCTGGTGAATGAGAAGATGATCCAGCCTGGCCAGTCTCGGAAGGACCTGCC
AGATCTGGGCTATGAGTTCGTGATGAGCTTTTACAAGAACGAGATCATCCAGTATGAGAAG
GACGGCAAGTTCTACAAGGAGAGGTTTCTGAGCCGCACCAAGCCCGCCTCCCGCAATTACA
TCGAGACAAAGCCCGTGGATAAGCCTAACTTCGAGAAGCGGCACCAGATCGGCCTGGCCAA
GACCACCTTCATCAGGAAGATCCGCACCGACATCCTGGGCAACGAATACAACTGCGATAGA
GAGAAGTTTTCCTCCATCTGCAAAAGGCCGGCGGCCACGAAAAAGGCCGGCCAGGCAAAAA
AGAAAAAGGGATCCTACCCATACGATGTTCCAGATTACGCTTATCCCTACGACGTGCCTGA
TTATGCATACCCATATGATGTCCCCGACTATGCC
(SEQ ID NO: 2)

NLS (bold), can be substituted with different NLSs
Linker (underlined), can be removed or extended
3×HA tag (italics), can be substituted with different tags

In some embodiments, recombinant engineered non-naturally occurring human codon-optimized Cas9 comprises a nucleic acid sequence having at least 60%, 70%, 80%, 85%, 90%, 95%, 97%, 98%, 99% sequence identity to SEQ ID NO: 2.

Various species exhibit codon bias (i.e. differences in codon usage by organisms) which correlates with the efficiency of translation of messenger RNA (mRNA) by utilizing codons in mRNA that correspond with the abundance of tRNA species for that codon in a particular organism. Various methods in the art can be used for computer optimization, including for example through use of software. In some embodiments, codon optimization refers to modification of nucleic acid sequences for enhanced expression in the host cells of interest by replacing at least one codon (e.g. 1, 2, 3, 4, 5, 10, 15, 20, 25, 50 or more codons) of the native sequence with codons that are more frequently used or most frequently used in the genes of the host cell while maintaining the native amino acid sequence.

In some embodiments, the Cas9 protein described herein is codon optimized. This type of optimization is known in the art and entails the mutation of foreign-derived DNA to mimic the codon preferences of the intended host organism or cell while encoding the same protein. Thus, the codons are changed, but the encoded protein remains unchanged. Codon optimization improves soluble protein levels and increases activity and editing efficiency in a given species. Codon optimization also results in increased translation and protein expression.

In some embodiments, the Cas9 protein is codon optimized for expression in eukaryotic cells. In some embodiments, the Cas9 protein is codon optimized for expression in human cells.

### Protospacer Adjacent Motif (PAM)

Each Cas endonuclease binds to its target sequence only in the presence of a specific sequence, known as a protospacer adjacent motif (PAM), on the non-targeted i.e. complementary DNA strand. Cas nucleases isolated from different bacterial species recognize different PAM sequences. For example, the SpCas9 nuclease (from Staphylococcus pyogenes) cuts upstream of the PAM sequence 5′-NGG-3′ (where “N” can be any nucleotide base), SaCas9 (from Staphylococcus aureus) recognizes the PAM sequence 5′-NNGRR (N)-3′ in the target. Thus, the locations in the genome that can be targeted by different Cas proteins are limited by the locations of unique PAM sequences.

The Cas9 protein described herein recognizes a PAM sequence defined by the following sequence 5′-NNGNG-3′. In some embodiments, the target nucleic acid is 5Y or upstream of the PAM sequence. Accordingly, the Cas9 protein described herein exhibits activity, for example, binding, cleavage, modification, or altered gene expression in the presence of a PAM sequence comprising 5′-NNGNG-3′.

In some embodiments, the Cas9 protein described herein does not bind or exhibit activity, for example, with the PAM sequences of 5′-NGG-3′.

### RNA Guides

An RNA guide comprises a polynucleotide sequence with complementarity to a target sequence. The RNA guide hybridizes with the target nucleic acid sequence and directs sequence-specific binding of a CRISPR complex to the target nucleic acid. In some embodiments, an RNA guide has 50%, 60%, 70%, 75%, 80%, 85%, 90%, 95%, 96%, 97%, 98%, 99% or 100% complementarity to a target nucleic acid sequence.

In some embodiments, the RNA guides are about 5, 10, 11, 12, 13, 14, 15, 16, 17, 18, 19, 20, 21, 22, 23, 24, 25, 26, 27, 28, 29, 30, 35, 40, 45, 50, 75 or more nucleotides in length. In some embodiments, the RNA guides are about 18-24 nucleotides in length. In some embodiments, the RNA guide is complementary to about 18-24 nucleotides in the target nucleic acid sequence. For example, the RNA guide is complementary to about 18, 19, 20, 21, 22, 23, or 24 nucleotides in the target nucleic acid sequence. In some embodiments, the RNA guide is complementary to about 18-22 nucleotides. In some embodiments, the RNA guide is complementary to about 18-21 nucleotides. In some embodiments, the RNA guide is complementary to about 18-20 nucleotides. In some embodiments, the RNA guide is complementary to 20 nucleotides in the target nucleic acid sequence.

An RNA guide can be designed to target any target sequence. Optimal alignment is determined using any algorithm for aligning sequences, including the Needleman-Wunsch algorithm, Smith-Waterman algorithm, Burrows-Wheeler algorithm, ClustlW, ClustlX, BLAST, Novoalign, SOAP, Maq, and ELAND.

In some embodiments, an RNA guide is targeted to a unique target sequence within the genome of a cell. In some embodiments, an RNA guide is designed to lack a PAM sequence. In some embodiments, an RNA guide sequence is designed to have optimal secondary structure using a folding algorithm including mFold or Geneious. In some embodiments, expression of RNA guides may be under an inducible promoter, e.g. hormone inducible, tetracycline or doxycycline inducible, arabinose inducible, or light inducible.

In some embodiments, the CRISPR system includes one or more RNA guides e.g. crRNA, tracrRNA, and/or sgRNA. Accordingly, in some embodiments the RNA guide comprises a crRNA. In some embodiments, the RNA guide comprises a tracrRNA. In some embodiments, the RNA guide comprises a sgRNA. In some embodiments, the CRISPR system includes multiple RNA guides, comprising 1, 2, 3, 4, 5, 6, 7, 8, 9, 10, 15 or more RNA guides.

In some embodiments, the RNA guide includes a crRNA. In some embodiments, the CRISPR system includes multiple crRNAs comprising 2-15 crRNAs. In some embodiments, the crRNA is a precursor crRNA (pre-crRNA), which includes a direct repeat sequence, a spacer sequence and a direct repeat sequence. In some embodiments, the crRNA is a processed or mature crRNA which includes a truncated direct repeat sequence.

In some embodiments, a CRISPR associated protein cleaves the pre-crRNA to form processed or mature crRNA.

In some embodiments, a CRISPR associated protein forms a complex with the mature crRNA and the spacer sequence targets the complex to a complementary sequence in the target nucleic acid. In some embodiments, an RNA guide comprises a direct repeat sequence and a spacer sequence capable of hybridizing under appropriate conditions to a target nucleic acid.

In some embodiments, the spacer length of crRNAs can range from about 15 to 50 nucleotides. In some embodiments, the spacer length of an RNA guide is at least 16 nucleotides, at least 17 nucleotides, at least 18 nucleotides, at least 19 nucleotides, at least 20 nucleotides, at least 21 nucleotides, or at least 22 nucleotides. In some embodiments, the spacer length is from 15 to 17 nucleotides (e.g., 15, 16, or 17 nucleotides), from 17 to 20 nucleotides (e.g., 17, 18, 19, or 20 nucleotides), from 20 to 24 nucleotides (e.g., 20, 21, 22, 23, or 24 nucleotides), from 23 to 25 nucleotides (e.g., 23, 24, or 25 nucleotides), from 24 to 27 nucleotides, from 27 to 30 nucleotides, from 30 to 45 nucleotides (e.g., 30, 31, 32, 33, 34, 35, 36, 37, 38, 39, 40, 41, 42, 43, 44, or 45 nucleotides), from 30 or 35 to 40 nucleotides, from 41 to 45 nucleotides, from 45 to 50 nucleotides (e.g., 45, 46, 47, 48, 49, or 50 nucleotides), or longer.

In some embodiments, the RNA guide comprises a direct repeat (DR) sequence of between about 16 and 26 nucleotides long. For example, in some embodiments, the DR is about 16 nucleotides long. In some embodiments, the DR is about 17 nucleotides long. In some embodiments, the DR is about 18 nucleotides long. In some embodiments, the DR is about 19 nucleotides long. In some embodiments, the DR is about 20 nucleotides long. In some embodiments, the DR is about 21 nucleotides long. In some embodiments, the DR is about 22 nucleotides long. In some embodiments, the DR is about 23 nucleotides long. In some embodiments, the DR is about 24 nucleotides long. In some embodiments, the DR is about 25 nucleotides long. In some embodiments, the DR is about 26 nucleotides long.

In some embodiments, the crRNA comprises a nucleotide guide sequence and a DR sequence. The nucleotide guide sequence can be between about 18 and 24 nucleotides long. Accordingly, in some embodiments, the nucleotide guide sequence is about 18 nucleotides long. In some embodiments, the nucleotide guide sequence is about 19 nucleotides long. In some embodiments, the nucleotide guide sequence is about 20 nucleotides long. In some embodiments, the nucleotide guide sequence is about 21 nucleotides long. In some embodiments, the nucleotide guide sequence is about 22 nucleotides long. In some embodiments, the crRNA comprises a nucleotide guide sequence of about 22 nucleotides long and a direct repeat of about 22 nucleotides long.

In some embodiments, crRNA comprises a full length direct repeat sequence that has a sequence identity of about 80% identity to AUUUUAGUUCCUGGAUAAUUCAAGUUAGUGUAAAAC (SEQ ID NO: 3). In some embodiments, the full length direct repeat has about 80%, 81%, 82%, 83%, 84%, 85%, 86%, 87%, 88%, 89%, 90%, 91%, 92%, 93%, 94%, 95%, 96%, 97%, 98%, 99% or more identical to SEQ ID NO: 3. In some embodiments, the full length direct repeat has a sequence that is identical to SEQ ID NO: 3. In some embodiments, the crRNA comprises a DR sequence comprising a 22 nt direct repeat sequence that has about 80% sequence identity to AUUUUAGUUCCUGGAUAAUUCA (SEQ ID NO: 4). In some embodiments, the crRNA comprises a 22 nucleotide sequence that has 80%, 81%, 82%, 83%, 84%, 85%, 86%, 87%, 88%, 89%, 90%, 91%, 92%, 93%, 94%, 95%, 96%, 97%, 98%, 99% or more identical to SEQ ID NO: 4. In some embodiments, the crRNA comprises a 22 nucleotide sequence that is identical to SEQ ID NO: 4.

In some embodiments, mature crRNA comprises a sequence of

(SEQ ID NO: 5)
NNNNNNNNNNNNNNNNNNNNAUUUUAGUUCCUGGAUAAUUCA.

In some embodiments, the crRNA comprises a sequence that has 1, 2, 3, 4, 5, 6, 7, 8, or 9 nucleotide changes in comparison to SEQ ID NO: 5.

In some embodiments, the crRNA sequences can be modified to “dead crRNAs,” “dead guides,” or “dead guide sequences” that can form a complex with a CRISPR-associated protein and bind specific targets without any substantial nuclease activity.

In some embodiments, the crRNA may be chemically modified in the sugar phosphate backbone or base. In some embodiments, the crRNA maybe modified using 2′O-methyl, 2′-F or locked nucleic acids to improve nuclease resistance or base pairing. In some embodiments, the crRNA may contain modified bases such as 2-thiouridine or N6-methyladenosine.

In some embodiments, the crRNA is conjugated with other oligonucleotides, peptides, proteins, tags, dyes, or polyethylene glycol.

In some embodiments, the crRNA may include aptamer or riboswitch sequences that can bind specific target molecules due to their three-dimensional structure.

In some embodiments, a trans-activating RNA (tracrRNA) is associated with crRNA to facilitate formation of a complex with Cas9 protein. In some embodiments, the tracrRNA sequence is about or more than about 5, 6, 7, 8, 9, 10, 11, 12, 13, 14, 15, 16, 17, 18, 19, 20, 25, 30, 40, 50, 60, 70, 80, 90, 100 or more nucleotides in length. In some embodiments, the tracrRNA is about 70 nucleotides in length.

In some embodiments, the tracrRNA comprises a sequence that has about 80% sequence identity to UGAAUUAUUCAGACCAACUAAAACAAGGCUUUAUGCCGAAAUCAAGGACACC UUCGGGUGUCCUUUUUU (SEQ ID NO: 6). In some embodiments, the tracrRNA comprises a sequence that is about 80%, 81%, 82%, 83%, 84%, 85%, 86%, 87%, 88%, 89%, 90%, 91%, 92%, 93%, 94%, 95%, 96%, 97%, 98%, 99% or more identical to SEQ ID NO: 6. In some embodiments, the tracrRNA comprises a sequence that is identical to SEQ ID NO: 6.

In some embodiments, the tracrRNA and crRNA are contained in a single transcript called single guide RNA (sgRNA). In some embodiments, the sgRNA includes a loop between the tracrRNA and sgRNA.

In some embodiments, the loop forming sequences are 3, 4, 5 or more nucleotides in length. In some embodiments, the loop has the sequence GAAA, AAAG, CAAA and/or AAAC.

In some embodiments, the tracrRNA and crRNA form a hairpin loop. In some embodiments, sgRNA has at least two or more hairpins. In some embodiments, sgRNA has two, three, four or five hairpins.

In some embodiments, sgRNA includes a transcription termination sequence, which includes a polyT sequences comprising six nucleotides. In some embodiments, the sgRNA comprises a tracrRNA that has one or more point mutations to break a 6×T stretch which acts as a U6 termination signal. For example, in some embodiments, the sgRNA comprises a tracrRNA that has one point mutation. In some embodiments, the sgRNA comprises a tracr RNA that has two point mutations. In some embodiments, the sgRNA comprises a tracrRNA that has three point mutations. In some embodiments, the sgRNA comprises a tracrRNA that has four point mutations. In some embodiments, the sgRNA comprises a tracrRNA that has five point mutations. In some embodiments, the sgRNA comprises a tracrRNA that has five point mutations.

In some embodiments, the sgRNA comprises 6 U (6×U) in the tracrRNA which will act as a U6 termination sequence. In some embodiments, the sgRNA comprises 5 U (5×U) in the tracrRNA which will act as a termination sequence. In some embodiments the sgRNA comprises 6 U (6×U) in the tracrRNA which will act as a termination sequence. In some embodiments, the sgRNA comprises at least 6 U (6×U) in the tracrRNA which will act as a termination sequence. In some embodiments, the sgRNA does not comprise a termination signal. In some embodiments, the sgRNA comprises a cleavage sequence. In some embodiments, the cleavage sequence is placed at the 5′ or 3′ end of the sgRNA. In some embodiments, the cleavage sequence is placed at the 5′ end of the sgRNA. In some embodiments, the cleavage sequence is placed at the 3′ end of the sgRNA. In some embodiments, the cleavage sequence is placed between the 5′ and 3′ end of the sgRNA.

In some embodiments, the sgRNA comprises a sequence having at least 80% identity to

(SEQ ID NO: 7)
AUUUUAGUUCCUGGAUAUAAUUAUUCAGACCAACUAAAACAAGGCUUUA
UGCCGAAAUCAAGGACACCUUCGGGUGUCCUUUUUUCUUUUU.

wherein the direct repeat 22 nt crRNA is in bold, and the tetra loop connecting the direct repeat with the tracrRNA is underlined. In some embodiments, the sgRNA comprises a sequence having 80%, 81%, 82%, 83%, 84%, 85%, 86%, 87%, 88%, 89%, 90%, 91%, 92%, 93%, 94%, 95%, 96%, 97%, 98%, 99% or more identity to SEQ ID NO: 7. In some embodiments, the sgRNA comprises a sequence identical to SEQ ID NO: 7.

In some embodiments, the sgRNA comprises a sequence having at least 80% identity to: AUUUUAGUUCCUGGAUAAUUGAAAUGAAUUAUUCAGACCAACUAAAACAAGG CUUUAUGCCGAAAUCAAGGACACCUUCGGGUGUCCUUUUUU (SEQ ID NO: 13; sgRNA-1). In some embodiments, the sgRNA comprises a sequence having 80%, 81%, 82%, 83%, 84%, 85%, 86%, 87%, 88%, 89%, 90%, 91%, 92%, 93%, 94%, 95%, 96%, 97%, 98%, 99% or more identity to SEQ ID NO: 13. In some embodiments, the sgRNA comprises a sequence identical to SEQ ID NO: 13.

In some embodiments, the sgRNA comprises a sequence identity having at least 80% identity to: AUUUUAGUUCCUGGAUAAUUCAAAUUAUUCAGACCAACUAAAACAAGGCUUU AUGCCGAAAUCAAGGACACCUUCGGGUGUCCUUUUUUCUUUUUAAGGAGGAA UAG (SEQ ID NO: 14; sgRNA-2). In some embodiments, the sgRNA comprises a sequence having 80%, 81%, 82%, 83%, 84%, 85%, 86%, 87%, 88%, 89%, 90%, 91%, 92%, 93%, 94%, 95%, 96%, 97%, 98%, 99% or more identity to SEQ ID NO: 14. In some embodiments, the sgRNA comprises a sequence identical to SEQ ID NO: 14.

In some embodiments, the sgRNA comprises a sequence identity having at least 80% identity to: AUUUUAGUUCCUGGAUAAUUCAAAUUAUUCAGACCAACUAAAACAAGGCUUU AUGCCGAAAUCAAGGACACCUUCGGGUGUCCUUUGUUCUUUAUAAGGAGCAA UAG (SEQ ID NO: 15; sgRNA-3). In some embodiments, the sgRNA comprises a sequence having 80%, 81%, 82%, 83%, 84%, 85%, 86%, 87%, 88%, 89%, 90%, 91%, 92%, 93%, 94%, 95%, 96%, 97%, 98%, 99% or more identity to SEQ ID NO: 15. In some embodiments, the sgRNA comprises a sequence identical to SEQ ID NO: 15.

In some embodiments, the sgRNA comprises a sequence identity having at least 80% identity to: AUUUUAGUUCCUGGUAAUUCAGACCAACUAAAACAAGGCUUUAUGCCGAAAU CAAGGACACCUUCGGGUGUCCUUUUUUCUUUUU (SEQ ID NO: 16; sg-RNA-4). In some embodiments, the sgRNA comprises a sequence having 80%, 81%, 82%, 83%, 84%, 85%, 86%, 87%, 88%, 89%, 90%, 91%, 92%, 93%, 94%, 95%, 96%, 97%, 98%, 99% or more identity to SEQ ID NO: 16. In some embodiments, the sgRNA comprises a sequence identical to SEQ ID NO: 16.

In some embodiments, the sgRNA comprises a sequence identity having at least 80% identity to: AUUUUAGUUCCUGGUAAUUCAGACCAACUAAAACAAGGCUUUAUGCCGAAAU CAAGGACACCUUCGGGUGUCCUUCUUUCUUUUU (SEQ ID NO: 17; sgRNA-5). In some embodiments, the sgRNA comprises a sequence having 80%, 81%, 82%, 83%, 84%, 85%, 86%, 87%, 88%, 89%, 90%, 91%, 92%, 93%, 94%, 95%, 96%, 97%, 98%, 99% or more identity to SEQ ID NO: 17. In some embodiments, the sgRNA comprises a sequence identical to SEQ ID NO: 17.

In some embodiments, the sgRNA comprises a sequence identity having at least 80% identity to: AUUUUAGUUCCUGGAUAAUUGAAAAAUUAUUCAGACCAACUAAAACAAGGCU UUAUGCCGAAAUCAAGGACACCUUCGGGUGUCCUUUUUU (SEQ ID NO: 18; sgRNA-6). In some embodiments, the sgRNA comprises a sequence having 80%, 81%, 82%, 83%, 84%, 85%, 86%, 87%, 88%, 89%, 90%, 91%, 92%, 93%, 94%, 95%, 96%, 97%, 98%, 99% or more identity to SEQ ID NO: 18. In some embodiments, the sgRNA comprises a sequence identical to SEQ ID NO: 18.

In some embodiments, the sgRNA comprises a sequence identity having at least 80% identity to: AUUUUAGUUCCUGGAUAAUGAAAAUUAUUCAGACCAACUAAAACAAGGCUUU AUGCCGAAAUCAAGGACACCUUCGGGUGUCCUUUUUU (SEQ ID NO: 19; sgRNA-7). In some embodiments, the sgRNA comprises a sequence having 80%, 81%, 82%, 83%, 84%, 85%, 86%, 87%, 88%, 89%, 90%, 91%, 92%, 93%, 94%, 95%, 96%, 97%, 98%, 99% or more identity to SEQ ID NO: 19. In some embodiments, the sgRNA comprises a sequence identical to SEQ ID NO: 19.

In some embodiments, the sgRNA comprises a sequence identity having at least 80% identity to: AUUUUAGUUCCUGGAUAAGAAAUUAUUCAGACCAACUAAAACAAGGCUUUAU GCCGAAAUCAAGGACACCUUCGGGUGUCCUUUUUU (SEQ ID NO: 20; sgRNA-8).

In some embodiments, the sgRNA comprises a sequence having 80%, 81%, 82%, 83%, 84%, 85%, 86%, 87%, 88%, 89%, 90%, 91%, 92%, 93%, 94%, 95%, 96%, 97%, 98%, 99% or more identity to SEQ ID NO: 20. In some embodiments, the sgRNA comprises a sequence identical to SEQ ID NO: 20.

In some embodiments, the sgRNA comprises a sequence identity having at least 80% identity to: AUUUUAGUUCCUGGAUAGAAAUAUUCAGACCAACUAAAACAAGGCUUUAUGC CGAAAUCAAGGACACCUUCGGGUGUCCUUUUUU (SEQ ID NO: 21; sgRNA-9). In some embodiments, the sgRNA comprises a sequence having 80%, 81%, 82%/0, 83%, 84%, 85%, 86%, 87%, 88%, 89%, 90%, 91%, 92%, 93%, 94%, 95%, 96%, 97%, 98%, 99% or more identity to SEQ ID NO: 21. In some embodiments, the sgRNA comprises a sequence identical to SEQ ID NO: 21.

In some embodiments, the sgRNA comprises a sequence identity having at least 80% identity to: AUUUUAGUUCCUGGAUGAAAAUUCAGACCAACUAAAACAAGGCUUUAUGCCG AAAUCAAGGACACCUUCGGGUGUCCUUUUUU (SEQ ID NO: 22; sgRNA-10). In some embodiments, the sgRNA comprises a sequence having 80%, 81%, 82%, 83%, 84%, 85%, 86%, 87%, 88%, 89%, 90%, 91%, 92%, 93%, 94%, 95%, 96%, 97%, 98%, 99% or more identity to SEQ ID NO: 22. In some embodiments, the sgRNA comprises a sequence identical to SEQ ID NO: 22.

In some embodiments, the sgRNA comprises a sequence identity having at least 80% identity to: AUUUUAGUUCCUGGAGAAAUUCAGACCAACUAAAACAAGGCUUUAUGCCGAA AUCAAGGACACCUUCGGGUGUCCUUUUUU (SEQ ID NO: 23; sgRNA-11). In some embodiments, the sgRNA comprises a sequence having 80%, 81%, 82%, 83%, 84%, 85%, 86%, 87%, 88%, 89%, 90%, 91%, 92%, 93%, 94%, 95%, 96%, 97%, 98%, 99% or more identity to SEQ ID NO: 23. In some embodiments, the sgRNA comprises a sequence identical to SEQ ID NO: 23.

In some embodiments, the tracrRNA is a separate transcript, not contained with crRNA sequence in the same transcript. Cas9 Fusion Proteins

In some embodiments, the Cas9 enzyme is fused to one or more heterologous protein domains. In some embodiments, the Cas9 enzyme is fused to more than about 1, 2, 3, 4, 5, 6, 7, 8, 9, 10 or more protein domains. In some embodiments, the heterologous protein domain is fused to the C-terminus of the Cas9 enzyme. In some embodiments, the heterologous protein domain is fused to the N-terminus of the Cas9 enzyme. In some embodiments, the heterologous protein domain is fused internally, between the C-terminus and the N-terminus of the Cas9 enzyme. In some embodiments, the internal fusion is made within the Cas9 RuvCI, RuvC II, RuvCIII, HNH, REC I, or PAM interacting domain.

A Cas9 protein may be directly or indirectly linked to another protein domain. In some embodiments, a suitable CRISPR system contains a linker or spacer that joins a Cas9 protein and a heterologous protein. An amino acid linker or spacer is generally designed to be flexible or to interpose a structure, such as an alpha-helix, between the two protein moieties. A linker or spacer can be relatively short, or can be longer. Typically, a linker or spacer contains for example 1-100 (e.g., 1-100, 5-100, 10-100, 20-100 30-100, 40-100, 50-100, 60-100, 70-100, 80-100, 90-100, 5-55, 10-50, 10-45, 10-40, 10-35, 10-30, 10-25, 10-20) amino acids in length. In some embodiments, a linker or spacer is equal to or longer than 1, 2, 3, 4, 5, 6, 7, 8, 9, 10, 15, 20, 25, 30, 35, 40, 45, 50, 55, 60, 65, 70, 75, 80, 85, 90, 95, or 100 amino acids in length. Typically, a longer linker may decrease steric hindrance. In some embodiments, a linker will comprise a mixture of glycine and serine residues. In some embodiments, the linker may additionally comprise threonine, proline and/or alanine residues.

In some embodiments, a Cas9 protein is fused to cellular localization signals, epitope tags, reporter genes, and protein domains with enzymatic activity, epigenetic modifying activity, RNA cleavage activity, nucleic acid binding activity, transcription modulation activity. In some embodiments, the Cas9 protein is fused to a nuclear localization sequence (NLS), a FLAG tag, a HIS tag, and/or a HA tag.

Suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, demyristoylation activity, integrase activity, transposase activity, recombinase activity, polymerase activity, ligase activity, helicase activity, or nuclease activity, any of which can modify DNA or a DNA-associated polypeptide (e.g., a histone or DNA binding protein). In some embodiments, the Cas9 protein is fused to a histone demethylase, a transcriptional activator or a deaminase.

Further suitable fusion partners include, but are not limited to boundary elements (e.g., CTCF), proteins and fragments thereof that provide periphery recruitment (e.g., Lamin A, Lamin B, etc.), and protein docking elements (e.g., FKBP/FRB, Pill/Abyl, etc.).

In particular embodiments, a Cas9 is fused to a cytidine or adenosine deaminase domain, e.g., for use in base editing. In some embodiments, the terms “cytidine deaminase” and “cytosine deaminase” can be used interchangeably. In certain embodiments, the cytidine deaminase domain may have sequence identity of 70%, 75%, 80%, 85%, 90%, 91%, 92%, 93%, 94%, 95%, 96%, 97%, 98%, 99% or more to any cytidine deaminase described herein. In some embodiments, the cytidine deaminase domain has cytidine deaminase activity, (e.g., converting C to U). In certain embodiments, the adenosine deaminase domain may have sequence identity of 70%, 75%, 80%, 85%, 90%, 91%, 92%, 93%, 94%, 95%, 96%, 97%, 98%, 99% or more to any adenosine deaminase described herein. In some embodiments, the adenosine deaminase domain has adenosine deaminase activity, (e.g., converting A to I). In some embodiments, the terms “adenosine deaminase” and “adenine deaminase” can be used interchangeably.

In some embodiments, a cytidine deaminase can comprise all or a portion of an apolipoprotein B mRNA editing complex (APOBEC) family deaminase. APOBEC is a family of evolutionarily conserved cytidine deaminases. Members of this family are C-to-U editing enzymes. The N-terminal domain of APOBEC like proteins is the catalytic domain, while the C-terminal domain is a pseudocatalytic domain. More specifically, the catalytic domain is a zinc dependent cytidine deaminase domain and is important for cytidine deamination. APOBEC family members include APOBEC1, APOBEC2, APOBEC3A, APOBEC3B, APOBEC3C, APOBEC3D (“APOBEC3E” now refers to this), APOBEC3F, APOBEC3G, APOBEC3H, APOBEC4, and Activation-induced (cytidine) deaminase. In some embodiments, a deaminase incorporated into a fusion protein comprises all or a portion of an APOBEC1 deaminase. In some embodiments, a deaminase incorporated into a fusion protein comprises all or a portion of APOBEC2 deaminase. In some embodiments, a deaminase incorporated into a fusion protein comprises all or a portion of is an APOBEC3 deaminase. In some embodiments, a deaminase incorporated into a fusion protein comprises all or a portion of an APOBEC3A deaminase. In some embodiments, a deaminase incorporated into a fusion protein comprises all or a portion of APOBEC3B deaminase. In some embodiments, a deaminase incorporated into a fusion protein comprises all or a portion of APOBEC3C deaminase. In some embodiments, a deaminase incorporated into a fusion protein comprises all or a portion of APOBEC3D deaminase. In some embodiments, a deaminase incorporated into a fusion protein comprises all or a portion of APOBEC3E deaminase. In some embodiments, a deaminase incorporated into a fusion protein comprises all or a portion of APOBEC3F deaminase. In some embodiments, a deaminase incorporated into a fusion protein comprises all or a portion of APOBEC3G deaminase. In some embodiments, a deaminase incorporated into a fusion protein comprises all or a portion of APOBEC3H deaminase. In some embodiments, a deaminase incorporated into a fusion protein comprises all or a portion of APOBEC4 deaminase. In some embodiments, a deaminase incorporated into a fusion protein comprises all or a portion of activation-induced deaminase (AID). In some embodiments a deaminase incorporated into a fusion protein comprises all or a portion of cytidine deaminase 1 (CDA1). It should be appreciated that a fusion protein can comprise a deaminase from any suitable organism (e.g., a human or a rat). In some embodiments, a deaminase domain of a fusion protein is from a human, chimpanzee, gorilla, monkey, cow, dog, rat, or mouse. In some embodiments, the deaminase domain of the fusion protein is derived from rat (e.g., rat APOBEC1). In some embodiments, the deaminase domain is human APOBEC1. In some embodiments, the deaminase domain is pmCDA1.

In some embodiments, Lachnospira UBA3212 Cas9 comprises a D8A mutation (“LubCas9 (D8A)”). In some embodiments, the LubCas9 (D8A) comprises a ppAPOBEC1 cytidine deaminase fused to the N-terminus of LubCas9 (D8A). In some embodiments, the LubCas9 (D8A) Cas9 ppAPOBEC1 fusion further comprises a nuclear localization sequence (NLS), a linker sequence and a Uracil DNA glycosylase inhibitor (UGI) domain sequence. In some embodiments, the LubCas9 (D8A) ppAPOBEC1 fusion comprises a sequence at least 80% identical to the following sequence:

(SEQ ID NO: 24)
MPAAKRVKLDGTSEKGPSTGDPTLRRRIESWEFDVFYDPRELRKETCLL
YEIKWGMSRKIWRSSGKNTTNHVEVNFIKKFTSERRFHSSISCSITWFL
SWSPCWECSQAIREFLSQHPGVTLVIYVARLFWHMDQRNRQGLRDLVNS
GVTIQIMRASEYYHCWRNFVNYPPGDEAHWPQYPPLWMMLYALELHCII
LSLPPCLKISRRWQNHLAFFRLHLQNCHYQTIPPHILLATGLIHPSVTW
RLKSGGSSGGSSGSETPGTSESATPESSGGSSGGSPKKKRKVGSVNVGL
AIGIASVGVAVVDSESGEILEAVSDLFESAEANQNVDRRGFRQSRRLKR
RQYNRIHDFMKLWEEFGFVKPENINLNTVGLRVKSLTEQVTLDELYVIL
LSELKHRGISYLEDSEEVDGGSEYKEGLRINQRELQSKYPCEIQLERLK
IYGRYRGNFTVEIDGEKVGLSNVFTTGAYRKEIQQLLSIQKTYQSKLTD
DFINKYLEIFDRKRQYYVGPGNEKSRTDYGRYTTKKDAEGNYITDENIF
EKLIGKCSTYPEEMRAAGASYTAQEFNLLNDLNNLTIGGRKIEEEEKRA
IIETIKSSKVVNVEKIICKVTGEDAETITGARIDKDDKRIYHSFECYRK
LKKALETIEVKIEEYSREELDELARILTLNTEREGILGELEKSFLDLGE
EVIDCVIDFRRKNGPLFSKWQSFSLRLMNDIIPDMYEQPKEQMTLLTEM
GLMKSKKEIFKGMKYIPENVMRDDIYNPVVVRSVRIAVRALNAVIKKYG
EIDKVVIEMPRDRNTEEQKKRIDAENKRNREELPGIEKRILEEYGIKIT
SAHYRNHKQLGLKLKLWNEQGGICPYSGKTIDLERLLQNAGDYEVDHII
PLSISLDDSRNNKVLVYASENQKKGNQTPYAYLSSVQREWGWEQYRHYV
LSDLKKKKISSKKIENYLFMKDISKIDVVKGFIQRNLNDTRYASKVVLN
QIYRDYSDAKNPFLQYEMETGDCIRKYSKKHNGSRIVSLKYHDGEVNSC
ITLESFFKANEKETKVSVIRGSFTSLMRKNLKLDKSREESYAHHAVDAL
LIAYSKMGYDSYHKLQGEFIDFETGEILDSRMWETNLEPDILKGYLYGR
KWSEIRENIKIAESRVKYWHMTNKKCNRSLCNQTLYGTRTYDGKIYQIK
KIKDIRTPEGLKTFKDLVDKNKGDHLLMARNDPKTYEQILDVSHKYGFE
KGSQKVVLMSLNPYRMDVYKNCNDGKYYLIGLKQSDIKCEGRHYVIDEE
KYAKVLVNEKMIQPGQSRKDLPDLGYEFVMSFYKNEIIQYEKDGKFYKE
RFLSRTKPASRNYIETKPVDKPNFEKRHQIGLAKTTFIRKIRTDILGNE
YNCDREKFSSICKRPAATKKAGQAKKKKGSSGGSGGSGGSTNLSDIIEK
ETGKQLVIQESILMLPEEVEEVIGNKPESDILVHTAYDESTDENVMLLT
SDAPEYKPWALVIQDSNGENKIKMLSGGSGGSGGSTNLSDIIEKETGKQ
LVIQESILMLPEEVEEVIGNKPESDILVHTAYDESTDENVMLLTSDAPE
YKPWALVIQDSNGENKIKML
NLS (bold, no underline or italics)

ppAPOBEC1 (italics and underlined, no bolding)
Linker (bold and underlined, no italics)
D8A mutation (bold and italics, no underlining)
UGI (italics, no underline or bolding)
LubCas9 (no bold, no italics or underlining)

Sequences of exemplary cytidine deaminases are provided below.

pmCDA1 (Petromyzon marinus)
(SEQ ID NO: 25)
MTDAEYVRIHEKLDIYTFKKQFFNNKKSVSHRCYVLFELKRRGERRACFWG
YAVNKPQSGTERGIHAEIFSIRKVEEYLRDNPGQFTINWYSSWSPCADCAE
KILEWYNQELRGNGHTLKIWACKLYYEKNARNQIGLWNLRDNGVGLNVMVS
EHYQCCRKIFIQSSHNQLNENRWLEKTLKRAEKRRSELSIMIQVKILHTTK
SPAV
Human AID:
(SEQ ID NO: 26)
MDSLLMNRRKFLYQFKNVRWAKGRRETYLCYVVKRRDSATSFSLDFGYLRN
KNGCHVELLFLRYISDWDLDPGRCYRVTWFTSWSPCYDCARHVADFLRGNP
NLSLRIFTARLYFCEDRKAEPEGLRRLHRAGVQIAIMTFKAPV
Human AID:
(SEQ ID NO: 27)
MDSLLMNRRKFLYQFKNVRWAKGRRETYLCYVVKRRDSATSFSLDFGYLRN
KNGCHVELLFLRYISDWDLDPGRCYRVTWFTSWSPCYDCARHVADFLRGNP
NLSLRIFTARLYFCEDRKAEPEGLRRLHRAGVQIAIMTFKDYFYCWNTFVE
NHERTFKAWEGLHENSVRLSRQLRRILLPLYEVDDLRDAFRTLGL
(underline: nuclear localization sequence; double
underline: nuclear export signal)
Mouse AID:
(SEQ ID NO: 28)
MDSLLMKQKKFLYHFKNVRWAKGRHETYLCYVVKRRDSATSCSLDFGHLRN
KSGCHVELLFLRYISDWDLDPGRCYRVTWFTSWSPCYDCARHVAEFLRWNP
NLSLRIFTARLYFCEDRKAEPEGLRRLHRAGVQIGIMTFKDYFYCWNTFVE
NRERTFKAWEGLHENSVRLTRQLRRILLPLYEVDDLRDAFRMLGF
(underline: nuclear localization sequence; double
underline: nuclear export signal)
Canine AID:
(SEQ ID NO: 29)
MDSLLMKQRKFLYHFKNVRWAKGRHETYLCYVVKRRDSATSFSLDFGHLRN
KSGCHVELLFLRYISDWDLDPGRCYRVTWFTSWSPCYDCARHVADFLRGYP
NLSLRIFAARLYFCEDRKAEPEGLRRLHRAGVQIAIMTFKDYFYCWNTFVE
NREKTFKAWEGLHENSVRLSRQLRRILLPLYEVDDLRDAFRTLGL
(underline: nuclear localization sequence; double
underline: nuclear export signal)
Bovine AID:
(SEQ ID NO: 30)
MDSLLKKQRQFLYQFKNVRWAKGRHETYLCYVVKRRDSPTSFSLDFGHLRN
KAGCHVELLFLRYISDWDLDPGRCYRVTWFTSWSPCYDCARHVADFLRGYP
NLSLRIFTARLYFCDKERKAEPEGLRRLHRAGVQIAIMTFKDYFYCWNTFV
ENHERTFKAWEGLHENSVRLSRQLRRILLPLYEVDDLRDAFRILGL
(underline: nuclear localization sequence; double
underline: nuclear export signal)
Rat AID:
(SEQ ID NO: 31)
MAVGSKPKAALVGPHWERERIWCFLCSTGLGTQQTGQTSRWLRPAATQDPV
SPPRSLLMKQRKFLYHFKNVRWAKGRHETYLCYVVKRRDSATSFSLDFGYL
RNKSGCHVELLFLRYISDWDLDPGRCYRVTWFTSWSPCYDCARHVADFLRG
NPNLSLRIFTARLTGWGALPAGLMSPARPSDYFYCWNTFVENHERTFKAWE
GLHENSVRLSRRLRRILLPLYEVDDLRDAFRTLGL
(underline: nuclear localization sequence; double
underline: nuclear export signal)
clAID (Canis lupus familiaris):
(SEQ ID NO: 32)
MDSLLMKQRKFLYHFKNVRWAKGRHETYLCYVVKRRDSATSFSLDFGHLRN
KSGCHVELLFLRYISDWDLDPGRCYRVTWFTSWSPCYDCARHVADFLRGYP
NLSLRIFAARLYFCEDRKAEPEGLRRLHRAGVQIAIMTFKDYFYCWNTFVE
NREKTFKAWEGLHENSVRLSRQLRRILLPLYEVDDLRDAFRTLGL
btAID (Bos Taurus):
(SEQ ID NO: 33)
MDSLLKKQRQFLYQFKNVRWAKGRHETYLCYVVKRRDSPTSFSLDFGHLRN
KAGCHVELLFLRYISDWDLDPGRCYRVTWFTSWSPCYDCARHVADFLRGYP
NLSLRIFTARLYFCDKERKAEPEGLRRLHRAGVQIAIMTFKDYFYCWNTFV
ENHERTFKAWEGLHENSVRLSRQLRRILLPLYEVDDLRDAFRTLGL
mAID (Mus musculus):
(SEQ ID NO: 34)
MDSLLMNRRKFLYQFKNVRWAKGRRETYLCYVVKRRDSATSFSLDFGYLRN
KNGCHVELLFLRYISDWDLDPGRCYRVTWFTSWSPCYDCARHVADFLRGNP
NLSLRIFTARLYFCEDRKAEPEGLRRLHRAGVQIAIMTFKDYFYCWNTFVE
NHERTFKAWEGLHENSVRLSRQLRRILLPLYEVDDLRDAFRTLGL
rAPOBEC-1 (Rattus norvegicus):
(SEQ ID NO: 35)
MSSETGPVAVDPTLRRRIEPHEFEVFFDPRELRKETCLLYEINWGGRHSIW
RHTSQNTNKHVEVNFIEKFTTERYFCPNTRCSITWFLSWSPCGECSRAITE
FLSRYPHVTLFIYIARLYHHADPRNRQGLRDLISSGVTIQIMTEQESGYCW
RNFVNYSPSNEAHWPRYPHLWVRLYVLELYCIILGLPPCLNILRRKQPQLT
FFTIALQSCHYQRLPPHILWATGLK
maAPOBEC-1 (Mesocricetus auratus):
(SEQ ID NO: 36)
MSSETGPVVVDPTLRRRIEPHEFDAFFDQGELRKETCLLYEIRWGGRHNIW
RHTGQNTSRHVEINFIEKFTSERYFYPSTRCSIVWFLSWSPCGECSKAITE
FLSGHPNVTLFIYAARLYHHTDQRNRQGLRDLISRGVTIRIMTEQEYCYCW
RNFVNYPPSNEVYWPRYPNLWMRLYALELYCIHLGLPPCLKIKRRHQYPLT
FFRLNLQSCHYQRIPPHILWATGFI
ppAPOBEC-1 (Pongo pygmaeus):
(SEQ ID NO: 37)
MTSEKGPSTGDPTLRRRIESWEFDVFYDPRELRKETCLLYEIKWGMSRKIW
RSSGKNTINHVEVNFIKKFTSERRFHSSISCSITWFLSWSPCWECSQAIRE
FLSQHPGVTLVIYVARLFWHMDQRNRQGLRDLVNSGVTIQIMRASEYYHCW
RNFVNYPPGDEAHWPQYPPLWMMLYALELHCIILSLPPCLKISRRWQNHLA
FFRLHLQNCHYQTIPPHILLATGLIHPSVTWR
ocAPOBEC1 (Oryctolagus cuniculus):
(SEQ ID NO: 38)
MASEKGPSNKDYTLRRRIEPWEFEVFFDPQELRKEACLLYEIKWGASSKTW
RSSGKNTINHVEVNFLEKLTSEGRLGPSTCCSITWFLSWSPCWECSMAIRE
FLSQHPGVTLIIFVARLFQHMDRRNRQGLKDLVTSGVTVRVMSVSEYCYCW
ENFVNYPPGKAAQWPRYPPRWMLMYALELYCIILGLPPCLKISRRHQKQLT
FFSLTPQYCHYKMIPPYILLATGLLQPSVPWR
mdAPOBEC-1 (Monodelphis domestica):
(SEQ ID NO: 39)
MNSKTGPSVGDATLRRRIKPWEFVAFFNPQELRKETCLLYEIKWGNQNIW
RHSNQNTSQHAEINFMEKFTAERHFNSSVRCSITWFLSWSPCWECSKAIR
KFLDHYPNVTLAIFISRLYWHMDQQHRQGLKELVHSGVTIQIMSYSEYHY
CWRNFVDYPQGEEDYWPKYPYLWIMLYVLELHCHILGLPPCLKISGSHSN
QLALFSLDLQDCHYQKIPYNVLVATGLVQPFVTWR
ppAPOBEC-2 (Pongo pygmaeus):
(SEQ ID NO: 40)
MAQKEEAAAATEAASQNGEDLENLDDPEKLKELIELPPFEIVTGERLPANF
FKFQFRNVEYSSGRNKTFLCYVVEAQGKGGQVQASRGYLEDEHAAAHAEEA
FFNTILPAFDPALRYNVTWYVSSSPCAACADRUIKTLSKTKNLRLLILVGR
LFMWEELEIQDALKKLKEAGCKLRIMKPQDFEYVWQNFVEQEEGESKAFQP
WEDIQENFLYYEEKLADILK
btAPOBEC-2 (Bos Taurus):
(SEQ ID NO: 41)
MAQKEEAAAAAEPASQNGEEVENLEDPEKLKELIELPPFEIVTGERLPAHY
FKFQFRNVEYSSGRNKTFLCYVVEAQSKGGQVQASRGYLEDEHATNHAEEA
FFNSIMPTFDPALRYMVTWYVSSSPCAACADRIVKTLNKTKNLRLLILVGR
LFMWEEPEIQAALRKLKEAGCRLRIMKPQDFEYIWQNFVEQEEGESKAFEP
WEDIQENFLYYEEKLADILK
mAPOBEC-3-(1) (Mus musculus):
(SEQ ID NO: 42)
MQPQRLGPRAGMGPFCLGCSHRKCYSPIRNLISQETFKFHFKNLGYAKGRK
DTFLCYEVTRKDCDSPVSLHHGVFKNKDNIHAEICFLYWFHDKVLKVLSPR
EEFKITWYMSWSPCFECAEQIVRFLATHHNLSLDIFSSRLYNVQDPETQQN
LCRLVQEGAQVAAMDLYEFKKCWKKFVDNGGRRFRPWKRLLTNFRYQDSKL
QEILRPCYISVPSSSSSTLSNICLTKGLPETRFWVEGRRMDPLSEEEFYSQ
FYNQRVKHLCYYHRMKPYLCYQLEQFNGQAPLKGCLLSEKGKQHAEILFLD
KIRSMELSQVTITCYLTWSPNCAWQLAAFKRDRPDLILHIYTSRLYFHWKR
PFQKGLCSLWQSGILVDVMDLPQFTDCWTNFVNPKRPFWPWKGLEIISRRT
QRRLRRIKESWGLQDLVNDFGNLQLGPPMS
Mouse APOBEC-3-(2):
(SEQ ID NO: 43)
MGPFCLGCSHRKCYSPIRNLISQETFKFHFKNLGYAKGRKDTFLCYEVTRK
DCDSPVSLHHGVFKNKDNIHAEICFLYWFHDKVLKVLSPREEFKITWYMSW
SPCFECAEQIVRFLATHHNLSLDIFSSRLYNVQDPETQQNLCRLVQEGAQV
AAMDLYEFKKCWKKFVDNGGRRFRPWKRLLTNFRYQDSKLQEILRPCYIPV
PSSSSSTLSNICLTKGLPETRFCVEGRRMDPLSEEEFYSQFYNQRVKHLCY
YHRMKPYLCYQLEQFNGQAPLKGCLLSEKGKQHAEILFLDKIRSMELSQVT
ITCYLTWSPCPNCAWQLAAFKRDRPDLILHIYTSRLYFHWKRPFQKGLCSL
WQSGILVDVDLPQFTDCWTNFVNPKRPFWPWKGLEIISRRTQRRLRRIMKE
SWGLQDLVNDFGNLQLGPPMS
(italic: nucleic acid editing domain)
Rat APOBEC-3:
(SEQ ID NO: 44)
MGPFCLGCSHRKCYSPIRNLISQETFKFHFKNRLRYAIDRKDTFLCYEVTR
KDCDSPVSLHHGVFKNKDNIHAEICFLYWFHDKVLKVLSPREEFKITWYMS
WSPCFECAEQVLRFLATHHNLSLDIFSSRLYNIRDPENQQNLCRLVQEGAQ
VAAMDLYEFKKCWKKFVDNGGRRFRPWKKLLTNFRYQDSKLQEILRPCYIP
VPSSSSSTLSNICLTKGLPETRFCVERRRVHLLSEEEFYSQFYNQRVKHLC
YYHGVKPYLCYQLEQFNGQAPLKGCLLSEKGKQHAEILFLDKIRSMEKSQV
IITCYLTWSPCPNCAWQLAAFKRDRPDLILHIYTSRLYFHWKRPFQKGLCS
LWQSGILVDVMDLPQFTDCWTNFVNPKRPFWPWKGLEIISRRTQRRLHRIK
ESWGLQDLVNDFGNLQLGPPMS
(italic: nucleic acid editing domain)
hAPOBEC-3A (Homo sapiens):
(SEQ ID NO: 45)
MEASPASGPRHLMDPHIFTSNFNNGIGRHKTYLCYEVERLDNGTSVKMDQH
RGFLHNQAKNLLCGFYGRHAELRFLDLVPSLQLDPAQIYRVTWFISWSPCF
SWGCAGEVRAFLQENTHVRLRIFAARIYDYDPLYKEALQMLRDAGAQVSIM
TYDEFKHCWDTFVDHQGCPFQPWDGLDEHSQALSGRLRAILQNQGN
hAPOBEC-3F (Homo sapiens):
(SEQ ID NO: 46)
MKPHFRNTVERMYRDTFSYNFYNRPILSRRNTVWLCYEVKTKGPSRPRLDA
KIFRGQVYSQPEHHAEMCFLSWFCGNQLPAYKCFQITWFVSWTPCPDCVAK
LAEFLAEHPNVTLTISAARLYYYWERDYRRALCRLSQAGARVKIMDDEEFA
YCWENFVYSEGQPFMPWYKFDDNYAFLHRTLKEILRNPMEAMYPHIFYFHF
KNLRKAYGRNESWLCFTMEVVKHHSPVSWKRGVFRNQVDPETHCHAERCFL
SWFCDDILSPNTNYEVTWYTSWSPCPECAGEVAEFLARHSNVNLTIFTARL
YYFWDTDYQEGLRSLSQEGASVEIMGYKDFKYCWENFVYNDDEPFKPWKGL
KYNFLFLDSKLQEILE
Rhesus macaque APOBEC-3G:
(SEQ ID NO: 47)
MVEPMDPRTFVSNFNNRPILSGLNTVWLCCEVKTKDPSGPPLDAKIFQGKV
YSKAKYHPEMRFLRWFHKWRQLHHDQEYKVTWYVSWSPCTRCANSVATFLA
KDPKVTLTIFVARLYYFWKPDYQQALRILCQKRGGPHATMKIMNYNEFQDC
WNKFVDGRGKPFKPRNNLPKHYTLLQATLGELLRHLMDPGTFTSNFNNKPW
VSGQHETYLCYKVERLHNDTWVPLNQHRGFLRNQAPNIHGFPKGRHAELCF
LDLIPFWKLDGQQYRVTCFTSWSPCFSCAQEMAKFISNNEHVSLCIFAARI
YDDQGRYQEGLRALHRDGAKIAMMNYSEFEYCWDTFVDRQGRPFQPWDGLD
EHSQALSGRLRAI
(italic: nucleic acid editing domain; underline:
cytoplasmic localization signal)
Chimpanzee APOBEC-3G:
(SEQ ID NO: 48)
MKPHFRNPVERMYQDTFSDNFYNRPILSHRNTVWLCYEVKTKGPSRPPLDA
KIFRGQVYSKLKYHPEMRFFHWFSKWRKLHRDQEYEVTWYISWSPCTKCTR
DVATFLAEDPKVTLTIFVARLYYFWDPDYQEALRSLCQKRDGPRATMKIMN
YDEFQHCWSKFVYSQRELFEPWNNLPKYYILLHIMLGEILRHSMDPPTFTS
NFNNELWVRGRHETYLCYEVERLHNDTWVLLNQRRGFLCNQAPHKHGFLEG
RHAELCFLDVIPFWKLDLHQDYRVTCFTSWSPCFSCAQEMAKFISNNKHVS
LCIFAARIYDDQGRCQEGLRTLAKAGAKISIMTYSEFKHCWDTFVDHQGCP
FQPWDGLEEHSQALSGRLRAILQNQGN
(italic: nucleic acid editing domain; underline:
cytoplasmic localization signal)
Green monkey APOBEC-3G:
(SEQ ID NO: 49)
MNPQIRNMVEQMEPDIFVYYFNNRPILSGRNTVWLCYEVKTKDPSGPPLDA
NIFQGKLYPEAKDHPEMKFLHWFRKWRQLHRDQEYEVTWYVSWSPCTRCAN
SVATFLAEDPKVTLTIFVARLYYFWKPDYQQALRILCQERGGPHATMKIMN
YNEFQHCWNEFVDGQGKPFKPRKNLPKHYTLLHATLGELLRHVMDPGTFTS
NFNNKPWVSGQRETYLCYKVERSHNDTWVLLNQHRGFLRNQAPDRHGFPKG
RHAELCFLDLIPFWKLDDQQYRVTCFTSWSPCFSCAQKMAKFISNNKHVSL
CIFAARIYDDQGRCQEGLRTLHRDGAKIAVMNYSEFEYCWDTFVDRQGRPF
QPWDGLDEHSQALSGRLRAI
(italic: nucleic acid editing domain; underline:
cytoplasmic localization signal)
Human APOBEC-3G:
(SEQ ID NO: 50)
MKPHFRNTVERMYRDTFSYNFYNRPILSRRNTVWLCYEVKTKGPSRPPLDA
KIFRGQVYSELKYHPEMRFFHWFSKWRKLHRDQEYEVTWYISWSPCTKCTR
DMATFLAEDPKVTLTIFVARLYYFWDPDYQEALRSLCQKRDGPRATMKIMN
YDEFQHCWSKFVYSQRELFEPWNNLPKYYILLHIMLGEILRHSMDPPTFTF
NFNNEPWVRGRHETYLCYEVERMHNDTWVLLNQRRGFLCNQAPHKHGFLEG
RHAELCFLDVIPFWKLDLDQDYRVTCFTSWSPCFSCAQEMAKFISKNKHVS
LCIFTARIYDDQGRCQEGLRTLAEAGAKISIMTYSEFKHCWDTFVDHQGCP
FQPWDGLDEHSQDLSGRLRAILQNQEN
(italic: nucleic acid editing domain; underline:
cytoplasmic localization signal)
Human APOBEC-3F:
(SEQ ID NO: 51)
MKPHFRNTVERMYRDTFSYNFYNRPILSRRNTVWLCYEVKTKGPSRPRLDA
KIFRGQVYSQPEHHAEMCFLSWFCGNQLPAYKCFQITWFVSWTPCPDCVAK
LAEFLAEHPNVTLTISAARLYYYWERDYRRALCRLSQAGARVKIMDDEEFA
YCWENFVYSEGQPFMPWYKFDDNYAFLHRTLKEILRNPMEAMYPHIFYFHF
KNLRKAYGRNESWLCFTMEVVKHHSPVSWKRGVFRNQVDPETHCHAERCFL
SWFCDDILSPNTNYEVTWYTSWSPCPECAGEVAEFLARHSNVNLTIFTARL
YYFWDTDYQEGLRSLSQEGASVEIMGYKDFKYCWENFVYNDDEPFKPWKGL
KYNFLFLDSKLQEILE
(italic: nucleic acid editing domain)
Human APOBEC-3B:
(SEQ ID NO: 52)
MNPQIRNPMERMYRDTFYDNFENEPILYGRSYTWLCYEVKIKRGRSNLLWD
TGVFRGQVYFKPQYHAEMCFLSWFCGNQLPAYKCFQITWFVSWTPCPDCVA
KLAEFLSEHPNVTLTISAARLYYYWERDYRRALCRLSQAGARVTIMDYEEF
AYCWENFVYNEGQQFMPWYKFDENYAFLHRTLKEILRYLMDPDTFTFNFNN
DPLVLRRRQTYLCYEVERLDNGTWVLMDQHMGFLCNEAKNLLCGFYGRHAE
LRFLDLVPSLQLDPAQIYRVTWFISWSPCFSWGCAGEVRAFLQENTHVRLR
IFAARIYDYDPLYKEALQMLRDAGAQVSIMTYDEFEYCWDTFVYRQGCPFQ
PWDGLEEHSQALSGRLRAILQNQGN
(italic: nucleic acid editing domain)
Rat APOBEC-3B:
(SEQ ID NO: 53)
MQPQGLGPNAGMGPVCLGCSHRRPYSPIRNPLKKLYQQTFYFHFKNVRYAW
GRKNNFLCYEVNGMDCALPVPLRQGVFRKQGHIHAELCFIYWFHDKVLRVL
SPMEEFKVTWYMSWSPCSKCAEQVARFLAAHRNLSLAIFSSRLYYYLRNPN
YQQKLCRLIQEGVHVAAMDLPEFKKCWNKFVDNDGQPFRPWMRLRINFSFY
DCKLQEIFSRMNLLREDVFYLQFNNSHRVKPVQNRYYRRKSYLCYQLERAN
GQEPLKGYLLYKKGEQHVEILFLELCTLWRSGIHVDVMDLPQFADCWTNFV
NPQRPFRPWNELEKNSWRIQRRLRRIKESWGL
Bovine APOBEC-3B:
(SEQ ID NO: 54)
MDGWEVAFRSGTVLKAGVLGVSMTEGWAGSGHPGQGACVWTPGTRNTMNLL
REVLFKQQFGNQPRVPAPYYRRKTYLCYQLKQRNDLTLDRGCFRNKKQRHA
ERFIDKINSLDLNPSQSYKIICYITWSPCPNCANELVNFITRNNHLKLEIF
ASRLYFHWIKSFKMGLQDLQNAGISVAVMTHTEFEDCWEQFVDNQSRPFQP
WDKLEQYSASIRRRLQRILTAPI
Chimpanzee APOBEC-3B:
(SEQ ID NO: 56)
MNPQIRNPMEWMYQRTFYYNFENEPILYGRSYTWLCYEVKIRRGHSNLLWD
TGVFRGQMYSQPEHHAEMCFLSWFCGNQLSAYKCFQITWFVSWTPCPDCVA
KLAKFLAEHPNVTLTISAARLYYYWERDYRRALCRLSQAGARVKIMDDEEF
AYCWENFVYNEGQPFMPWYKFDDNYAFLHRTLKEIIRHLMDPDTFTFNFNN
DPLVLRRHQTYLCYEVERLDNGTWVLMDQHMGFLCNEAKNLLCGFYGRHAE
LRFLDLVPSLQLDPAQIYRVTWFISWSPCFSWGCAGQVRAFLQENTHVRLR
IFAARIYDYDPLYKEALQMLRDAGAQVSIMTYDEFEYCWDTFVYRQGCPFQ
PWDGLEEHSQALSGRLRAILQVRASSLCMVPHRPPPPPQSPGPCLPLCSEP
PLGSLLPTGRPAPSLPFLLTASFSFPPPASLPPLPSLSLSPGHLPVPSFHS
LTSCSIQPPCSSRIRETEGWASVSKEGRDLG
Human APOBEC-3C:
(SEQ ID NO: 57)
MNPQIRNPMKAMYPGTFYFQFKNLWEANDRNETWLCFTVEGIKRRSVVSWK
TGVFRNQVDSETHCHAERCFLSWFCDDILSPNTKYQVTWYTSWSPCPDCAG
EVAEFLARHSNVNLTIFTARLYYFQYPCYQEGLRSLSQEGVAVEIMDYEDF
KYCWENFVYNDNEPFKPWKGLKTNFRLLKRRLRESLQ
(italic: nucleic acid editing domain)
Gorilla APOBEC-3C
(SEQ ID NO: 58)
MNPQIRNPMKAMYPGTFYFQFKNLWEANDRNETWLCFTVEGIKRRSVVSWK
TGVFRNQVDSETHCHAERCFLSWECDDILSPNTNYQVTWYTSWSPCPECAG
EVAEFLARHSNVNLTIFTARLYYFQDTDYQEGLRSLSQEGVAVKIMDYKDF
KYCWENFVYNDDEPFKPWKGLKYNFRFLKRRLQEILE
(italic: nucleic acid editing domain)
Human APOBEC-3A:
(SEQ ID NO: 59)
MEASPASGPRHLMDPHIFTSNFNNGIGRHKTYLCYEVERLDNGTSVKMDQH
RGFLHNQAKNLLCGFYGRHAELRFLDLVPSLQLDPAQIYRVTWFISWSPCF
SWGCAGEVRAFLQENTHVRLRIFAARIYDYDPLYKEALQMLRDAGAQVSIM
TYDEFKHCWDTFVDHQGCPFQPWDGLDEHSQALSGRLRAILQNQGN
(italic: nucleic acid editing domain)
Rhesus macaque APOBEC-3A:
(SEQ ID NO: 60)
MDGSPASRPRHLMDPNTFTFNFNNDLSVRGRHQTYLCYEVERLDNGTWVPM
DERRGFLCNKAKNVPCGDYGCHVELRFLCEVPSWQLDPAQTYRVTWFISWS
PCFRRGCAGQVRVFLQENKHVRLRIFAARIYDYDPLYQEALRTLRDAGAQV
SIMTYEEFKHCWDTFVDRQGRPFQPWDGLDEHSQALSGRLRAILQNQGN
(italic: nucleic acid editing domain)
Bovine APOBEC-3A:
(SEQ ID NO: 61)
MDEYTFTENFNNQGWPSKTYLCYEMERLDGDATIPLDEYKGFVRNKGLDQP
EKPCHAELYFLGKIHSWNLDRNQHYRLTCFISWSPCYDCAQKLTTFLKENH
HISLHILASRIYTHNRFGCHQSGLCELQAAGARITIMTFEDFKHCWETFVD
HKGKPFQPWEGLNVKSQALCTELQAILKTQQN
(italic: nucleic acid editing domain)
Human APOBEC-3H:
(SEQ ID NO: 62)
MALLTAETFRLQFNNKRRLRRPYYPRKALLCYQLTPQNGSTPTRGYFENKK
KCHAEICFINEIKSMGLDETQCYQVTCYLTWSPCSSCAWELVDFIKAHDHL
NLGIFASRLYYHWCKPQQKGLRLLCGSQVPVEVMGFPKFADCWENFVDHEK
PLSFNPYKMLEELDKNSRAIKRRLERIKIPGVRAQGRYMDILCDAEV
(italic: nucleic acid editing domain)
Rhesus macaque APOBEC-3H:
(SEQ ID NO: 63)
MALLTAKTFSLQFNNKRRVNKPYYPRKALLCYQLTPQNGSTPTRGHLKNKK
KDHAEIRFINKIKSMGLDETQCYQVTCYLTWSPCPSCAGELVDFIKAHRHL
NLRIFASRLYYHWRPNYQEGLLLLCGSQVPVEVMGLPEFTDCWENFVDHKE
PPSFNPSEKLEELDKNSQAIKRRLERIKSRSVDVLENGLRSLQLGPVTPSS
SIRNSR
Human APOBEC-3D:
(SEQ ID NO: 64)
MNPQIRNPMERMYRDTFYDNFENEPILYGRSYTWLCYEVKIKRGRSNLLWD
TGVFRGPVLPKRQSNHRQEVYFRFENHAEMCFLSWFCGNRLPANRRFQITW
FVSWNPCLPCVVKVTKFLAEHPNVTLTISAARLYYYRDRDWRWVLLRLHKA
GARVKIMDYEDFAYCWENFVCNEGQPFMPWYKFDDNYASLHRTLKEILRNP
MEAMYPHIFYFHFKNLLKACGRNESWLCFTMEVTKHHSAVFRKRGVFRNQV
DPETHCHAERCFLSWFCDDILSPNTNYEVTWYTSWSPCPECAGEVAEFLAR
HSNVNLTIFTARLCYFWDTDYQEGLCSLSQEGASVKIMGYKDFVSCWKNFV
YSDDEPFKPWKGLQTNFRLLKRRLREILQ
(italic: nucleic acid editing domain)
Human APOBEC-1:
(SEQ ID NO: 65)
MTSEKGPSTGDPTLRRRIEPWEFDVFYDPRELRKEACLLYEIKWGMSRKIW
RSSGKNTINHVEVNFIKKFTSERDFHPSMSCSITWFLSWSPCWECSQAIRE
FLSRHPGVTLVIYVARLFWHMDQQNRQGLRDLVNSGVTIQIMRASEYYHCW
RNFVNYPPGDEAHWPQYPPLWMMLYALELHCHILSLPPCLKISRRWQNHLT
FFRLHLQNCHYQTIPPHILLATGLIHPSVAWR
Mouse APOBEC-1:
(SEQ ID NO: 66)
MSSETGPVAVDPTLRRRIEPHEFEVFFDPRELRKETCLLYEINWGGRHSVW
RHTSQNTSNHVEVNFLEKFTTERYFRPNTRCSITWFLSWSPCGECSRAITE
FLSRHPYVTLFIYIARLYHHTDQRNRQGLRDLISSGVTIQIMTEQEYCYCW
RNFVNYPPSNEAYWPRYPHLWVKLYVLELYCHILGLPPCLKILRRKQPQLT
FFTITLQTCHYQRIPPHLLWATGLK
Rat APOBEC-1:
(SEQ ID NO: 67)
MSSETGPVAVDPTLRRRIEPHEFEVFFDPRELRKETCLLYEINWGGRHSIW
RHTSQNTNKHVEVNFIEKFTTERYFCPNTRCSITWFLSWSPCGECSRAITE
FLSRYPHVTLFIYIARLYHHADPRNRQGLRDLISSGVTIQIMTEQESGYCW
RNFVNYSPSNEAHWPRYPHLWVRLYVLELYCIILGLPPCLNILRRKQPQLT
FFTIALQSCHYQRLPPHILWATGLK
Human APOBEC-2:
(SEQ ID NO: 68)
MAQKEEAAVATEAASQNGEDLENLDDPEKLKELIELPPFEIVTGERLPANF
FKFQFRNVEYSSGRNKTFLCYVVEAQGKGGQVQASRGYLEDEHAAAHAEEA
FFNTILPAFDPALRYNVTWYVSSSPCAACADRIIKTLSKTKNLRLLILVGR
LFMWEEPEIQAALKKLKEAGCKLRIMKPQDFEYVWQNFVEQEEGESKAFQP
WEDIQENFLYYEEKLADILK
Mouse APOBEC-2:
(SEQ ID NO: 69)
MAQKEEAAEAAAPASQNGDDLENLEDPEKLKELIDLPPFEIVTGVRLPVNF
FKFQFRNVEYSSGRNKTFLCYVVEVQSKGGQAQATQGYLEDEHAGAHAEEA
FFNTILPAFDPALKYNVTWYVSSSPCAACADRILKTLSKTKNLRLLILVSR
LFMWEEPEVQAALKKLKEAGCKLRIMKPQDFEYIWQNFVEQEEGESKAFEP
WEDIQENFLYYEEKLADILK
Rat APOBEC-2:
(SEQ ID NO: 70)
MAQKEEAAEAAAPASQNGDDLENLEDPEKLKELIDLPPFEIVTGVRLPVNF
FKFQFRNVEYSSGRNKTFLCYVVEAQSKGGQVQATQGYLEDEHAGAHAEEA
FFNTILPAFDPALKYNVTWYVSSSPCAACADRILKTLSKTKNLRLLILVSR
LFMWEEPEVQAALKKLKEAGCKLRIMKPQDFEYLWQNFVEQEEGESKAFEP
WEDIQENFLYYEEKLADILK
Bovine APOBEC-2:
(SEQ ID NO: 71)
MAQKEEAAAAAEPASQNGEEVENLEDPEKLKELIELPPFEIVTGERLPAHY
FKFQFRNVEYSSGRNKTFLCYVVEAQSKGGQVQASRGYLEDEHATNHAEEA
FFNSIMPTFDPALRYMVTWYVSSSPCAACADRIVKTLNKTKNLRLLILVGR
LFMWEEPEIQAALRKLKEAGCRLRIMKPQDFEYIWQNFVEQEEGESKAFEP
WEDIQENFLYYEEKLADILK
Petromyzon marinus CDA1 (pmCDA1):
(SEQ ID NO: 72)
MTDAEYVRIHEKLDIYTFKKQFFNNKKSVSHRCYVLFELKRRGERRACFWG
YAVNKPQSGTERGIHAEIFSIRKVEEYLRDNPGQFTINWYSSWSPCADCAE
KILEWYNQELRGNGHTLKIWACKLYYEKNARNQIGLWNLRDNGVGLNVMVS
EHYQCCRKIFIQSSHNQLNENRWLEKTLKRAEKRRSELSFMIQVKILHTTK
SPAV
Human APOBEC3G D316R D317R:
(SEQ ID NO: 73)
MKPHFRNTVERMYRDTFSYNFYNRPILSRRNTVWLCYEVKTKGPSRPPLDA
KIFRGQVYSELKYHPEMRFFHWFSKWRKLHRDQEYEVTWYISWSPCTKCTR
DMATFLAEDPKVTLTIFVARLYYFWDPDYQEALRSLCQKRDGPRATMKFNY
DEFQHCWSKFVYSQRELFEPWNNLPKYYILLHFMLGEILRHSMDPPTFTFN
FNNEPWVRGRHETYLCYEVERMHNDTWVLLNQRRGFLCNQAPHKHGFLEGR
HAELCFLDVIPFWKLDLDQDYRVTCFTSWSPCFSCAQEMAKFISKKHVSLC
IFTARIYRRQGRCQEGLRTLAEAGAKISFTYSEFKHCWDTFVDHQGCPFQP
WDGLDEHSQDLSGRLRAILQNQEN
Human APOBEC3G chain A:
(SEQ ID NO: 74)
MDPPTFTFNFNNEPWWGRHETYLCYEVERMHNDTWVLLNQRRGFLCNQAPH
KHGFLEGRHAELCFLDVIPFWKLDLDQDYRVTCFTSWSPCFSCAQEMAKFI
SKNKHVSLCIFTARIYDDQGRCQEGLRTLAEAGAKISFTYSEFKHCWDTFV
DHQGCPFQPWDGLDEHSQDLSGRLRAILQ
Human APOBEC3G chain A D120R D121R:
(SEQ ID NO: 75)
MDPPTFTFNFNNEPWVRGRHETYLCYEVERMHNDTWVLLNQRRGFLCNQAP
HKHGFLEGRHAELCFLDVIPFWKLDLDQDYRVTCFTSWSPCFSCAQEMAKF
ISKNKHVSLCIFTARIYRRQGRCQEGLRTLAEAGAKISFMTYSEFKHCWDT
FVDHQGCPFQPWDGLDEHSQDLSGRLRAILQ
hAPOBEC-4 (Homo sapiens):
(SEQ ID NO: 76)
MEPIYEEYLANHGTIVKPYYWLSFSLDCSNCPYHIRTGEEARVSLTEFCQI
FGFPYGTTFPQTKHLTFYELKTSSGSLVQKGHASSCTGNYIHPESMLFEMN
GYLDSAIYNNDSIRHIILYSNNSPCNEANHCCISKMYNFLITYPGITLSIY
FSQLYHTEMDFPASAWNREALRSLASLWPRVVLSPISGGIWHSVLHSFISG
VSGSHVFQPILTGRALADRHNAYEINAITGVKPYFTDVLLQTKRNPNTKAQ
EALESYPLNNAFPGQFFQMPSGQLQPNLPPDLRAPVVFVLVPLRDLPPMHM
GQNPNKPRNIVRHLNMPQMSFQETKDLGRLPTGRSVEIVEITEQFASSKEA
DEKKKKKGKK
mAPOBEC-4 (Mus musculus):
(SEQ ID NO: 77)
MDSLLMKQKKFLYHFKNVRWAKGRHETYLCYVVKRRDSATSCSLDFGHLRN
KSGCHVELLFLRYISDWDLDPGRCYRVTWFTSWSPCYDCARHVAEFLRWNP
NLSLRIFTARLYFCEDRKAEPEGLRRLHRAGVQIGIMTFKDYFYCWNTFVE
NRERTFKAWEGLHENSVRLTRQLRRILLPLYEVDDLRDAFRMLGF
rAPOBEC-4 (Rattus norvegicus):
(SEQ ID NO: 78)
MEPLYEEYLTHSGTIVKPYYWLSVSLNCTNCPYHIRTGEEARVPYTEFHQT
FGFPWSTYPQTKHLTFYELRSSSGNLIQKGLASNCTGSHTHPESMLFERDG
YLDSLIFHDSNIRHIILYSNNSPCDEANHCCISKMYNFLMNYPEVTLSVFF
SQLYHTENQFPTSAWNREALRGLASLWPQVTLSAISGGIWQSILETFVSGI
SEGLTAVRPFTAGRTLTDRYNAYEINCITEVKPYFTDALHSWQKENQDQKV
WAASENQPLHNTTPAQWQPDMSQDCRTPAVFMLVPYRDLPPIHVNPSPQKP
RTVVRHLNTLQLSASKVKALRKSPSGRPVKKEEARKGSTRSQEANETNKSK
WKKQTLFIKSNICHLLEREQKKIGILSSWSV
mfAPOBEC-4 (Macaca fascicularis):
(SEQ ID NO: 79)
MEPTYEEYLANHGTIVKPYYWLSFSLDCSNCPYHIRTGEEARVSLTEFCQI
FGFPYGTTYPQTKHLTFYELKTSSGSLVQKGHASSCTGNYIHPESMLFEMN
GYLDSAIYNNDSIRHIILYCNNSPCNEANHCCISKVYNFLITYPGITLSIY
FSQLYHTEMDFPASAWNREALRSLASLWPRVVLSPISGGIWHSVLHSFVSG
VSGSHVFQPILTGRALTDRYNAYEINAITGVKPFFTDVLLHTKRNPNTKAQ
MALESYPLNNAFPGQSFQMTSGIPPDLRAPVVFVLLPLRDLPPMHMGQDPN
KPRNIIRHLNMPQMSFQETKDLERLPTRRSVETVEITERFASSKQAEEKTK
KKKGKK
pmCDA-1 (Petromyzon marinus):
(SEQ ID NO: 80)
MAGYECVRVSEKLDFDIFEFQFENLHYATERHRTYVIFDVKPQSAGGRSRR
LWGYIINNPNVCHAELILMSMIDRHLESNPGVYAMTWYMSWSPCANCSSKL
NPWLKNLLEEQGHTLTMHFSRIYDRDREGDHRGLRGLKHVSNSFRMGVVGR
AEVKECLAEYVEASRRTLTWLDTTESMAAKMRRKLFCILVRCAGMRESGIP
LHLFTLQTPLLSGRVVWWRV
pmCDA-2 (Petromyzon marinus):
(SEQ ID NO: 81)
MELREVVDCALASCVRHEPLSRVAFLRCFAAPSQKPRGTVILFYVEGAGRG
VTGGHAVNYNKQGTSIHAEVLLLSAVRAALLRRRRCEDGEEATRGCTLHCY
STYSPCRDCVEYIQEFGASTGVRVVIHCCRLYELDVNRRRSEAEGVLRSLS
RLGRDFRLMGPRDAIALLLGGRLANTADGESGASGNAWVTETNVVEPLVDM
TGFGDEDLHAQVQRNKQIREAYANYASAVSLMLGELHVDPDKFPFLAEFLA
QTSVEPSGTPRETRGRPRGASSRGPEIGRQRPADFERALGAYGLFLHPRIV
SREADREEIKRDLIVVMRKHNYQGP
pmCDA-5 (Petromyzon marinus):
(SEQ ID NO: 82)
MAGDENVRVSEKLDFDTFEFQFENLHYATERHRTYVIFDVKPQSAGGRSRR
LWGYIINNPNVCHAELILMSMIDRHLESNPGVYAMTWYMSWSPCANCSSKL
NPWLKNLLEEQGHTLMMHFSRIYDRDREGDHRGLRGLKHVSNSFRMGVVGR
AEVKECLAEYVEASRRTLTWLDTTESMAAKMRRKLFCILVRCAGMRESGMP
LHLFT
yCD (Saccharomyces cerevisiae):
(SEQ ID NO: 83)
MVTGGMASKWDQKGMDIAYEEAALGYKEGGVPIGGCLINNKDGSVLGRGHN
MRFQKGSATLHGEISTLENCGRLEGKVYKDTTLYTTLSPCDMCTGAIIMYG
IPRCVVGENVNFKSKGEKYLQTRGHEVVVVDDERCKKIMKQFIDERPQDWF
EDIGE
rAPOBEC-1 (delta 177-186):
(SEQ ID NO: 84)
MSSETGPVAVDPTLRRRIEPHEFEVFFDPRELRKETCLLYEINWGGRHSIW
RHTSQNTNKHVEVNFIEKFTTERYFCPNTRCSITWFLSWSPCGECSRAITE
FLSRYPHVTLFIYIARLYHHADPRNRQGLRDLISSGVTIQIMTEQESGYCW
RNFVNYSPSNEAHWPRYPHLWVRGLPPCLNILRRKQPQLTFFTIALQSCHY
QRLPPHILWATGLK
rAPOBEC-1 (delta 202-213):
(SEQ ID NO: 85)
MSSETGPVAVDPTLRRRIEPHEFEVFFDPRELRKETCLLYEINWGGRHSIW
RHTSQNTNKHVEVNFIEKFTTERYFCPNTRCSITWFLSWSPCGECSRAITE
FLSRYPHVTLFIYIARLYHHADPRNRQGLRDLISSGVTIQIMTEQESGYCW
RNFVNYSPSNEAHWPRYPHLWVRLYVLELYCIILGLPPCLNILRRKQPQHY
QRLPPHILWATGLK
Mouse APOBEC-3:
(SEQ ID NO: 86)
MGPFCLGCSHRKCYSPIRNLISQETFKFHFKNLGYAKGRKDTFLCYEVTRK
DCDSPVSLHHGVFKNKDNIHAEICFLYWFHDKVLKVLSPREEFKITWYMSW
SPCFECAEQIVRFLATHHNLSLDIFSSRLYNVQDPETQQNLCRLVQEGAQV
AAMDLYEFKKCWKKFVDNGGRRFRPWKRLLTNFRYQDSKLQEILRPCYIPV
PSSSSSTLSNICLTKGLPETRFCVEGRRMDPLSEEEFYSQFYNQRVKHLCY
YHRMKPYLCYQLEQFNGQAPLKGCLLSEKGKQHAEILFLDKIRSMELSQVT
ITCYLTWSPCPNCAWQLAAFKRDRPDLILHIYTSRLYFHWKRPFQKGLCSL
WQSGILVDVMDLPQFTDCWTNFVNPKRPFWPWKGLEIISRRTQRRLRRIKE
SWGLQDLVNDFGNLQLGPPMS
(italic: nucleic acid editing domain)

In some embodiments, an adenosine deaminase can comprise all or a portion of an adenosine deaminase ADAR (e.g., ADAR1 or ADAR2). In another embodiment, an adenosine deaminase can comprise all or a portion of an adenosine deaminase ADAT. In some embodiments, an adenosine deaminase can comprise all or a portion of an ADAT from Escherichia coli (EcTadA) comprising one or more of the following mutations: D108N, A106V, D147Y, E155V, L84F, H123Y, I157F. or a corresponding mutation in another adenosine deaminase. The adenosine deaminase can be derived from any suitable organism (e.g., E. coli). In some embodiments, the adenosine deaminase is from Escherichia coli. Staphylococcus aureus, Salmonella typhi, Shewanella putrefaciens, Haemophilus influenzae, Caulobacter crescentus, or Bacillus subtilis. In some embodiments, the adenosine deaminase is from E. coli. In some embodiments, the adenine deaminase is a naturally-occurring adenosine deaminase that includes one or more mutations corresponding to any of the mutations provided herein (e.g., mutations in ecTadA). The corresponding residue in any homologous protein can be identified by e.g., sequence alignment and determination of homologous residues. The mutations in any naturally-occurring adenosine deaminase (e.g., having homology to ecTadA) that corresponds to any of the mutations described herein (e.g., any of the mutations identified in ecTadA) can be generated accordingly. In particular embodiments, the TadA is any one of the TadA described in PCT/US2017/045381 (WO 2018/027078), which is incorporated herein by reference in its entirety. Mutations were identified through rounds of evolution and selection (e.g., TadA*7.10=variant 10 from seventh round of evolution) having desirable adenosine deaminase activity on single stranded DNA as shown in Table 3.

TABLE 3
Genotypes of TadA Variants

| TadA | 23 | 26 | 36 | 37 | 48 | 49 | 51 | 72 | 84 | 87 | 105 | 108 | 123 | 125 | 142 | 145 | 147 | 152 | 155 | 156 | 157 | 16 |
|---|---|---|---|---|---|---|---|---|---|---|---|---|---|---|---|---|---|---|---|---|---|---|
| 0.1 | W | R | H | N | P | | R | N | L | S | A | D | H | G | A | S | D | R | E | I | K | K |
| 0.2 | W | R | H | N | P | | R | N | L | S | A | D | H | G | A | S | D | R | E | I | K | K |
| 1.1 | W | R | H | N | P | | R | N | L | S | A | N | H | G | A | S | D | R | E | I | K | K |
| 1.2 | W | R | H | N | P | | R | N | L | S | V | N | H | G | A | S | D | R | E | I | K | K |
| 2.1 | W | R | H | N | P | | R | N | L | S | V | N | H | G | A | S | Y | R | V | I | K | K |
| 2.2 | W | R | H | N | P | | R | N | L | S | V | N | H | G | A | S | Y | R | V | I | K | K |
| 2.3 | W | R | H | N | P | | R | N | L | S | V | N | H | G | A | S | Y | R | V | I | K | K |
| 2.4 | W | R | H | N | P | | R | N | L | S | V | N | H | G | A | S | Y | R | V | I | K | K |
| 2.5 | W | R | H | N | P | | R | N | L | S | V | N | H | G | A | S | Y | R | V | I | K | K |
| 2.6 | W | R | H | N | P | | R | N | L | S | V | N | H | G | A | S | Y | R | V | I | K | K |
| 2.7 | W | R | H | N | P | | R | N | L | S | V | N | H | G | A | S | Y | R | V | I | K | K |
| 2.8 | W | R | H | N | P | | R | N | L | S | V | N | H | G | A | S | Y | R | V | I | K | K |
| 2.9 | W | R | H | N | P | | R | N | L | S | V | N | H | G | A | S | Y | R | V | I | K | K |
| 2.10 | W | R | H | N | P | | R | N | L | S | V | N | H | G | A | S | Y | R | V | I | K | K |
| 2.11 | W | R | H | N | P | | R | N | L | S | V | N | H | G | A | S | Y | R | V | I | K | K |
| 2.12 | W | R | H | N | P | | R | N | L | S | V | N | H | G | A | S | Y | R | V | I | K | K |
| 3.1 | W | R | H | N | P | | R | N | F | S | V | N | Y | G | A | S | Y | R | V | F | K | K |
| 3.2 | W | R | H | N | P | | R | N | F | S | V | N | Y | G | A | S | Y | R | V | F | K | K |
| 3.3 | W | R | H | N | P | | R | N | F | S | V | N | Y | G | A | S | Y | R | V | F | K | K |
| 3.4 | W | R | H | N | P | | R | N | F | S | V | N | Y | G | A | S | Y | R | V | F | K | K |
| 3.5 | W | R | H | N | P | | R | N | F | S | V | N | Y | G | A | S | Y | R | V | F | K | K |
| 3.6 | W | R | H | N | P | | R | N | F | S | V | N | Y | G | A | S | Y | R | V | F | K | K |
| 3.7 | W | R | H | N | P | | R | N | F | S | V | N | Y | G | A | S | Y | R | V | F | K | K |
| 3.8 | W | R | H | N | P | | R | N | F | S | V | N | Y | G | A | S | Y | R | V | F | K | K |
| 4.1 | W | R | H | N | P | | R | N | L | S | V | N | H | G | N | S | Y | R | V | I | K | K |
| 4.2 | W | G | H | N | P | | R | N | L | S | V | N | H | G | N | S | Y | R | V | I | K | K |
| 4.3 | W | R | H | N | P | | R | N | F | S | V | N | Y | G | N | S | Y | R | V | F | K | K |
| 5.1 | W | R | L | N | P | | L | N | F | S | V | N | Y | G | A | C | Y | R | V | F | N | K |
| 5.2 | W | R | H | S | P | | R | N | F | S | V | N | Y | G | A | S | Y | R | V | F | K | T |
| 5.3 | W | R | L | N | P | | L | N | I | S | V | N | Y | G | A | C | Y | R | V | I | N | K |
| 5.4 | W | R | H | S | P | | R | N | F | S | V | N | Y | G | A | S | Y | R | V | F | K | T |
| 5.5 | W | R | L | N | P | | L | N | F | S | V | N | Y | G | A | C | Y | R | V | F | N | K |
| 5.6 | W | R | L | N | P | | L | N | F | S | V | N | Y | G | A | C | Y | R | V | F | N | K |
| 5.7 | W | R | L | N | P | | L | N | F | S | V | N | Y | G | A | C | Y | R | V | F | N | K |
| 5.8 | W | R | L | N | P | | L | N | F | S | V | N | Y | G | A | C | Y | R | V | F | N | K |
| 5.9 | W | R | L | N | P | | L | N | F | S | V | N | Y | G | A | C | Y | R | V | F | N | K |
| 5.10 | W | R | L | N | P | | L | N | F | S | V | N | Y | G | A | C | Y | R | V | F | N | K |
| 5.11 | W | R | L | N | P | | L | N | F | S | V | N | Y | G | A | C | Y | R | V | F | N | K |
| 5.12 | W | R | L | N | P | | L | N | F | S | V | N | Y | G | A | C | Y | R | V | F | N | K |
| 5.13 | W | R | H | N | P | | L | D | F | S | V | N | Y | A | A | S | Y | R | V | F | K | K |
| 5.14 | W | R | H | N | S | | L | N | F | C | V | N | Y | G | A | S | Y | R | V | F | K | K |
| 6.1 | W | R | H | N | S | | L | N | F | S | V | N | Y | G | N | S | Y | R | V | F | K | K |
| 6.2 | W | R | H | N | T | V | L | N | F | S | V | N | Y | G | N | S | Y | R | V | F | N | K |
| 6.3 | W | R | L | N | S | | L | N | F | S | V | N | Y | G | A | C | Y | R | V | F | N | K |
| 6.4 | W | R | L | N | S | | L | N | F | S | V | N | Y | G | N | C | Y | R | V | F | N | K |
| 6.5 | W | R | L | N | I | V | L | N | F | S | V | N | Y | G | A | C | Y | R | V | F | N | K |
| 6.6 | W | R | L | N | T | V | L | N | F | S | V | N | Y | G | N | C | Y | R | V | F | N | K |
| 7.1 | W | R | L | N | A | | L | N | F | S | V | N | Y | G | A | C | Y | R | V | F | N | K |
| 7.2 | W | R | L | N | A | | L | N | F | S | V | N | Y | G | N | C | Y | R | V | F | N | K |
| 7.3 | I | R | L | N | A | | L | N | F | S | V | N | Y | G | A | C | Y | R | V | F | N | K |
| 7.4 | R | R | L | N | A | | L | N | F | S | V | N | Y | G | A | C | Y | R | V | F | N | K |
| 7.5 | W | R | L | N | A | | L | N | F | S | V | N | Y | G | A | C | Y | H | V | F | N | K |
| 7.6 | W | R | L | N | A | | L | N | I | S | V | N | Y | G | A | C | Y | P | V | I | N | K |
| 7.7 | L | R | L | N | A | | L | N | F | S | V | N | Y | G | A | C | Y | P | V | F | N | K |
| 7.8 | I | R | L | N | A | | L | N | F | S | V | N | Y | G | N | C | Y | R | V | F | N | K |
| 7.9 | L | R | L | N | A | | L | N | F | S | V | N | Y | G | N | C | Y | P | V | F | N | K |
| 7.10 | R | R | L | N | A | | L | N | F | S | V | N | Y | G | A | C | Y | P | V | F | N | K |

In some embodiments, the TadA is provided as a monomer or dimer (e.g., a heterodimer of wild-type E. coli TadA and an engineered TadA variant). In some embodiments, the adenosine deaminase is an eighth generation TadA*8 variant as shown in Table 4 below.

TABLE 4
TadA8* Adenosine Deaminase Variants

| Adenosine | |
|---|---|
| Deaminase | Adenosine Deaminase Description |
| TadA*8.1 | Monomer_TadA*7.10 + Y147T |
| TadA*8.2 | Monomer_TadA*7.10 + Y147R |
| TadA*8.3 | Monomer_TadA*7.10 + Q154S |
| TadA*8.4 | Monomer_TadA*7.10 + Y123H |
| TadA*8.5 | Monomer_TadA*7.10 + V82S |
| TadA*8.6 | Monomer_TadA*7.10 + T166R |
| TadA*8.7 | Monomer_TadA*7.10 + Q154R |
| TadA*8.8 | Monomer_TadA*7.10 + Y147R_Q154R_Y123H |
| TadA*8.9 | Monomer_TadA*7.10 + Y147R_Q154R_I76Y |
| TadA*8.10 | Monomer_TadA*7.10 + Y147R_Q154R_T166R |
| TadA*8.11 | Monomer_TadA*7.10 + Y147T_Q154R |
| TadA*8.12 | Monomer_TadA*7.10 + Y147T_Q154S |
| TadA*8.13 | Monomer_TadA*7.10 + H123H_Y147R_Q154R_I76Y |
| TadA*8.14 | Heterodimer_(WT) + (TadA*7.10 + Y147T) |
| TadA*8.15 | Heterodimer_(WT) + (TadA*7.10 + Y147R) |
| TadA*8.16 | Heterodimer_(WT) + (TadA*7.10 + Q154S) |
| TadA*8.17 | Heterodimer_(WT) + (TadA*7.10 + Y123H) |
| TadA*8.18 | Heterodimer_(WT) + (TadA*7.10 + V82S) |
| TadA*8.19 | Heterodimer_(WT) + (TadA*7.10 + T166R) |
| TadA*8.20 | Heterodimer_(WT) + (TadA*7.10 + Q154R) |
| TadA*8.21 | Heterodimer_(WT) + (TadA*7.10 + |
| | Y147R_Q154R_Y123H) |
| TadA*8.22 | Heterodimer_(WT) + (TadA*7.10 + |
| | Y147R_Q154R_I76Y) |
| TadA*8.23 | Heterodimer_(WT) + (TadA*7.10 + |
| | Y147R_Q154R_T166R) |
| TadA*8.24 | Heterodimer_(WT) + (TadA*7.10 + |
| | Y147T_Q154R) |
| TadA*8.25 | Heterodimer_(WT) + (TadA*7.10 + |
| | Y147T_Q154S) |
| TadA*8.26 | Heterodimer_(WT) + (TadA*7.10 + |
| | H123H_Y147T_Q154R_I76Y) |

In some embodiments, the adenosine deaminase is a ninth generation TadA*9 variant containing an alteration at an amino acid position selected from the following: 21, 23, 25, 38, 51, 54, 70, 71, 72, 72, 94, 124, 133, 138, 139, 146, and 158 of a TadA variant as shown in the reference sequence below:

```
(SEQ ID NO: 87)
                10 20 30 40
MSEVEFSHEY WMRHALTLAK RARDEREVPV GAVLVLNNRV
                50 60 70 80
IGEGWNRAIG LHDPTAHAEI MALRQGGLVM QNYRLIDATL
                90 100 110 120
YVTFEPCVMC AGAMIHSRIG RVVFGVRNAK TGAAGSLMDV
              130 140 150 160
LHYPGMNHRV EITEGILADE CAALLCYFFR MPRQVFNAQK
KAQSSTD
```

In one embodiment, the adenosine deaminase variant contains alterations at two or more amino acid positions selected from the following: 21, 23, 25, 38, 51, 54, 70, 71, 72, 94, 124, 133, 138, 139, 146, and 158 of the TadA reference sequence above. In another embodiment, the adenosine deaminase variant contains one or more (e.g., 2, 3, 4) alterations selected from the following: R21N, R23H, E25F, N38G, L51W, P54C, M70V, Q71M, N72K, Y73S, M94V, P124W, T133K, D139L, D139M, C146R, and A158K of SEQ ID NO. 1. In other embodiments, the adenosine deaminase variant further contains one or more of the following alterations: Y147T, Y147R, Q154S, Y123H, and Q154R. In still other embodiments, the adenosine deaminase variant contains a combination of alterations relative to the above TadA reference sequence selected from the following:
E25F+V82S+Y123H, T133K+Y147R+Q154R;
E25F+V82S+Y123H+Y147R+Q154R; L51W+V82S+Y123H+C146R+Y147R+Q154R;
Y73S+V82S+Y123H+Y147R+Q154R;
P54C+V82S+Y123H+Y147R+Q154R;
N38G+V82T+Y123H+Y147R+Q154R;
N72K+V82S+Y123H+D139L+Y147R+Q154R;
E25F+V82S+Y123H+D139M+Y147R+Q154R;
Q71M+V82S+Y123H+Y147R+Q154R;
E25F+V82S+Y123H+T133K+Y147R+Q154R;
E25F+V82S+Y123H+Y147R+Q154R;
V82S+Y123H+P124W+Y147R+Q154R;
L51W+V82S+Y123H+C146R+Y147R+Q154R;
P54C+V82S+Y123H+Y147R+Q154R;
Y73S+V82S+Y123H+Y147R+Q154R;
N38G+V82T+Y123H+Y147R+Q154R;
R23H+V82S+Y123H+Y147R+Q154R;
R21N+V82S+Y123H+Y147R+Q154R;
V82S+Y123H+Y147R+Q154R+A158K;
N72K+V82S+Y123H+D139L+Y147R+Q154R;
E25F+V82S+Y123H+D139M+Y147R+Q154R;
M70V+V82S+M94V+Y123H+Y147R+Q154R;
Q71M+V82S+Y123H+Y147R+Q154R; E25F+I76Y+V82S+Y123H+Y147R+Q154R; I76Y+V82T+Y123H+Y147R+Q154R; N38G+I76Y+V82S+Y123H+Y147R+Q154R;
R23H+I76Y+V82S+Y123H+Y147R+Q154R;
P54C+I76Y+V82S+Y123H+Y147R+Q154R;
R21N+I76Y+V82S+Y123H+Y147R+Q154R;
I76Y+V82S+Y123H+D138M+Y147R+Q154R;
Y72S+I76Y+V82S+Y123H+Y147R+Q154R; E25F+I76Y+V82S+Y123H+Y147R+Q154R;
I76Y+V82T+Y123H+Y147R+Q154R;
N38G+I76Y+V82S+Y123H+Y147R+Q154R;
R23H+I76Y+V82S+Y123H+Y147R+Q154R;
P54C+I76Y+V82S+Y123H+Y147R+Q154R;
R21N+I76Y+V82S+Y123H+Y147R+Q154R;
I76Y+V82S+Y123H+D138M+Y147R+Q154R;
Y72S+I76Y+V82S+Y123H+Y147R+Q154R; and
V82S+Q154R;
N72K+V82S+Y123H+Y147R+Q154R;
Q71M+V82S+Y123H+Y147R+Q154R;
V82S+Y123H+T133K+Y147R+Q154R;
V82S+Y123H+T133K+Y147R+Q154R+A158K;
M70V+Q71M+N72K+V82S+Y123H+Y147R+Q154R;
N72K V82S+Y123H+Y147R+Q154R;
Q71M_V82S+Y123H+Y147R+Q154R; M70V+V82S+M94V+Y123H+Y147R+Q154R;
V82S+Y123H+T133K+Y147R+Q154R;
V82S+Y123H+T133K+Y147R+Q154R+A158K; and
M70V+Q71M+N72K+V82S+Y123H+Y147R+Q154R.
   In some embodiments, the deaminase or other polypeptide sequence lacks a methionine, for example when included as a component of a fusion protein. This can alter the numbering of positions. However, the skilled person will understand that such corresponding mutations refer to the same mutation, e.g., Y73S and Y72S and D139M and D138M.

In some embodiments, Cas9 is fused to nuclear localization sequences, including an NLS of the SV40 large T antigen, nucleoplasmin, c-myc, hRNPA1 M9, IBB domain from importin-alpha, NLS of myoma T protein, human p53, c-abl IV, influenza virus NS1, hepatitis virus delta antigen, mouse Mx1, human poly(ADP-ribose) polymerase, steroid hormone receptor (human) glucocorticoid.

In some embodiments, a Cas9 protein is fused to epitope tags including, but not limited to hemagglutinin (HA) tags, histidine (His) tags, FLAG tags, Myc tags, V5 tags, VSV-G tags, SNAP tags, thioredoxin (Trx) tags.

In some embodiments, Cas9 is fused to reporter genes including, but not limited to glutathione-S-transferase (GST), horseradish peroxidase (HRP), chloramphenicol transferase (CAT), HcRed, DsRed, cyan fluorescent protein, yellow fluorescent protein and blue fluorescent protein, green fluorescent protein (GFP), including enhanced versions or superfolded GFP, as well as other modified versions of reporter genes.

In some embodiments, serum half-life of an engineered Cas9 protein is increased by fusion with heterologous proteins such as a human serum albumin protein, transferrin protein, human IgG and/or sialylated peptide, such as the carboxy-terminal peptide (CTP, of chorionic gonadotropin β chain).

In some embodiments, serum half-life of an engineered Cas9 protein is decreased by fusion with destabilizing domains, including but not limited to geminin, ubiquitin, FKBP12-L106P, and/or dihydrofolate reductase.

Suitable fusion partners that provide for increased or decreased stability include, but are not limited to degron sequences. Degrons are readily understood by one of ordinary skill in the art to be amino acid sequences that control the stability of the protein of which they are part. For example, the stability of a protein comprising a degron sequence is controlled at least in part by the degron sequence. In some cases, a suitable degron is constitutive such that the degron exerts its influence on protein stability independent of experimental control (i.e., the degron is not drug inducible, temperature inducible, etc.) In some cases, the degron provides the variant Cas9 polypeptide with controllable stability such that the variant Cas9 polypeptide can be turned “on” (i.e., stable) or “off (i.e., unstable, degraded) depending on the desired conditions. For example, if the degron is a temperature sensitive degron, the variant Cas9 polypeptide may be functional (i.e., “on”, stable) below a threshold temperature (e.g., 42° C., 41° C., 40° C., 39° C., 38° C. 37° C., 36° C., 35° C. 34° C., 33° C., 32° C. 31° C., 30° C., etc.) but non-functional (i.e., “off, degraded) above the threshold temperature. As another example, if the degron is a drug inducible degron, the presence or absence of drug can switch the protein from an “off (i.e., unstable) state to an “on” (i.e., stable) state or vice versa. An exemplary drug inducible degron is derived from the FKBP12 protein. The stability of the degron is controlled by the presence or absence of a small molecule that binds to the degron.

Examples of suitable degrons include, but are not limited to those degrons controlled by Shield-1, DHFR, auxins, and/or temperature. Non-limiting examples of suitable degrons are known in the art (e.g., Dohmen et al., Science, 1994. 263(5151): p. 1273-1276: Heat-inducible degron: a method for constructing temperature-sensitive mutants; Schoeber et al., Am J Physiol Renal Physiol. 2009 January; 296(1):F204-11: Conditional fast expression and function of multimeric TRPV5 channels using Shield-1; Chu et al., Bioorg Med Chem Lett. 2008 Nov. 15; 18(22):5941-4: Recent progress with FKBP-derived destabilizing domains; Kanemaki, Pflugers Arch. 2012 Dec. 28: Frontiers of protein expression control with conditional degrons; Yang et al., Mol Cell. 2012 Nov. 30:48(4):487-8; Titivated for destruction: the methyl degron; Barbour et al., Biosci Rep. 2013 Jan. 18; 33(1). Characterization of the bipartite degron that regulates ubiquitin-independent degradation of thymidylate synthase; and Greussing et al., J Vis Exp. 2012 Nov. 10; (69): Monitoring of ubiquitin-proteasome activity in living cells using a Degron (dgn)-destabilized green fluorescent protein (GFP)-based reporter protein; all of which are hereby incorporated in their entirety by reference).

Exemplary degron sequences have been well-characterized and tested in both cells and animals. Thus, fusing dead Cas9 to a degron sequence produces a “tunable” and “inducible” dead Cas9 polypeptide.

Any of the fusion partners described herein can be used in any desirable combination. As one non-limiting example to illustrate this point, a Cas9 fusion protein can comprise a YFP sequence for detection, a degron sequence for stability, and transcription activator sequence to increase transcription of the target DNA. Furthermore, the number of fusion partners that can be used in a dCas9 fusion protein is unlimited. In some cases, a Cas9 fusion protein comprises one or more (e.g. two or more, three or more, four or more, or five or more) heterologous sequences.

### Target Nucleic Acids

A target nucleic acid is a DNA molecule, RNA molecule, which is single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases either deoxyribonucleotides, ribonucleotides, or analogs thereof. Target nucleic acids may have three-dimensional structure, may include coding or non-coding regions, may include exons, introns, mRNA, tRNA, rRNA, siRNA, shRNA, miRNA, ribozymes, cDNA, plasmids, vectors, exogenous sequences, endogenous sequences. A target nucleic acid can comprise modified nucleotides, include methylated nucleotides, or nucleotide analogs. In some embodiments, a target nucleic acid may be interspersed with non-nucleic acid components.

A target nucleic acid is recognized by CRISPR-Cas9 system and binds Cas9. In some embodiments, it is modified or cleaved or has altered expression due to the binding of Cas9. A target nucleic acid contains a specific recognizable PAM motif, for example, 5′-NNGNG-3′.

### Recombinant Gene Technology

In accordance with the present disclosure, there may be employed conventional molecular biology, microbiology, and recombinant DNA techniques within the skill of the art. Such techniques are described in the literature (see, e.g., Sambrook, Fritsch & Maniatis, Molecular Cloning: A Laboratory Manual, Second Edition (1989) Cold Spring Harbor Laboratory Press, Cold Spring Harbor, N.Y.; DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells and Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994).

Recombinant expression of a gene, such as a nucleic acid encoding a polypeptide, such as an engineered Cas9 enzyme described herein, can include construction of an expression vector containing a nucleic acid that encodes the polypeptide. Once a polynucleotide has been obtained, a vector for the production of the polypeptide can be produced by recombinant DNA technology using techniques known in the art. Known methods can be used to construct expression vectors containing polypeptide coding sequences and appropriate transcriptional and translational control signals. These methods include, for example, in vitro recombinant DNA techniques, synthetic techniques, and in vivo genetic recombination.

An expression vector can be transferred to a host cell by conventional techniques, and the transfected cells can then be cultured by conventional techniques to produce polypeptides.

In some embodiments, a nucleotide sequence encoding a DNA-targeting RNA and/or Cas9 protein is operably linked to a control element, e.g., a transcriptional control element, such as a promoter. The transcriptional control element may be functional in either a eukaryotic cell, e.g., a mammalian cell; or a prokaryotic cell (e.g., bacterial or archaeal cell). In some embodiments, the eukaryotic cell is a human cell. In some embodiments, a nucleotide sequence encoding a DNA-targeting RNA and/or a novel Cas9 protein is operably linked to multiple control elements that allow expression of the encoded nucleotide sequence in both prokaryotic and eukaryotic cells.

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein), it may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31(17)), and/or a human HI promoter (HI).

Examples of inducible promoters include, but are not limited to T7 RNA polymerase promoter, T3 RNA polymerase promoter, Isopropyl-beta-D-thiogalactopyranoside (IPTG)-regulated promoter, lactose induced promoter, heat shock promoter, Tetracycline-regulated promoter (e.g., Tet-ON, Tet-OFF, etc.), Steroid-regulated promoter, Metal-regulated promoter, estrogen receptor-regulated promoter, etc. Inducible promoters can therefore be regulated by molecules including, but not limited to, doxycycline, RNA polymerase, e.g., T7 RNA polymerase, an estrogen receptor and/or an estrogen receptor fusion.

In some embodiments, the promoter is a spatially restricted promoter (i.e., cell type specific promoter, tissue specific promoter, etc.) such that in a multi-cellular organism, the promoter is active (i.e., “ON”) in a subset of specific cells. Spatially restricted promoters may also be referred to as enhancers, transcriptional control elements, control sequences, etc. Any convenient spatially restricted promoter may be used and the choice of suitable promoter (e.g., a brain specific promoter, a promoter that drives expression in a subset of neurons, a promoter that drives expression in the germline, a promoter that drives expression in the lungs, a promoter that drives expression in muscles, a promoter that drives expression in islet cells of the pancreas, etc.) will depend on the organism. Thus, a spatially restricted promoter can be used to regulate the expression of a nucleic acid encoding a subject site-directed polypeptide in a wide variety of different tissues and cell types, depending on the organism. Some spatially restricted promoters are also temporally restricted such that the promoter is in the “ON” state or “OFF” state during specific stages of embryonic development or during specific stages of a biological process (e.g., hair follicle cycle).

For illustration purposes, examples of spatially restricted promoters include, but are not limited to, neuron-specific promoters, adipocyte-specific promoters, cardiomyocyte-specific promoters, smooth muscle-specific promoters, photoreceptor-specific promoters, etc. Neuron-specific spatially restricted promoters include, but are not limited to, a neuron-specific enolase (NSE) promoter, an aromatic amino acid decarboxylase (AADC) promoter, a neurofilament promoter, a synapsin promoter, a thy-1 promoter, a serotonin receptor promoter, a tyrosine hydroxylase promoter (TH), a GnRH promoter, an L7 promoter, a DNMT promoter, an enkephalin promoter, a myelin basic protein (MBP) promoter, a Ca²⁺-calmodulin-dependent protein kinase 11-alpha (CamKIIa) promoter and/or a CMV enhancer/platelet-derived growth factor-O promoter.

Adipocyte-specific spatially restricted promoters include, but are not limited to aP2 gene promoter/enhancer, e.g., a region from −5.4 kb to +21 bp of a human aP2 gene, a glucose transporter-4 (GLUT4) promoter, a fatty acid translocase (FAT/CD36) promoter, a stearoyl-CoA desaturase-1 (SCD1) promoter, a leptin promoter, and an adiponectin promoter, an adipsin promoter and/or a resistin promoter.

Cardiomyocyte-specific spatially restricted promoters include, but are not limited to control sequences derived from the following genes: myosin light chain-2, a-myosin heavy chain, AE3, cardiac troponin C, and/or cardiac actin.

Smooth muscle-specific spatially restricted promoters include, but are not limited to an SM22a promoter, a smoothelin promoter, and/or an a-smooth muscle actin promoter.

Photoreceptor-specific spatially restricted promoters include, but are not limited to, a rhodopsin promoter, a rhodopsin kinase promoter, a beta phosphodiesterase gene promoter, a retinitis pigmentosa gene promoter, an interphotoreceptor retinoid-binding protein (IRBP) gene enhancer, and/or an IRBP gene promoter.

### Gene Editing Uses of CRISPR-Cas9

The CRISPR-Cas9 system described herein can be used for gene editing, which can result in a gene silencing event, or an alteration of the expression (e.g., an increase or a decrease) in the expression of a desired target gene. Accordingly, in some embodiments, the CRISPR-Cas9 system described herein is used in a method of altering the expression of a target nucleic acid. In some embodiments the CRISPR-Cas9 system described herein is used in a method of modifying a target nucleic acid in a desired target cell. In some embodiments, the invention provides methods for site-specific modification of a target nucleic acid in eukaryotic cells to effectuate a desired modification in gene expression.

In some embodiments, the invention provides an engineered, non-naturally occurring CRISPR-Cas system comprising: an RNA guide or a nucleic acid encoding the RNA guide, wherein the RNA guide comprises a direct repeat sequence and a spacer sequence capable of hybridizing to a target nucleic acid; and a codon-optimized CRISPR-associated (Cas) protein having at least 80% sequence identity to SEQ ID NO: 1, and wherein the Cas protein is capable of binding to the RNA guide and of causing a break in the target nucleic acid sequence complementary to the RNA guide.

In some embodiments, the invention provides engineered, non-naturally occurring CRISPR-Cas system comprising: an RNA guide or a nucleic acid encoding the RNA guide, wherein the RNA guide comprises a direct repeat sequence and a spacer sequence capable of hybridizing to a target nucleic acid; and a codon-optimized CRISPR-associated (Cas) protein having at least 80% sequence identity to SEQ ID NO: 1; wherein the Cas protein is fused to a deaminase, and wherein the Cas protein fusion is capable of binding to the RNA guide and of editing the target nucleic acid sequence complementary to the RNA guide.

In some embodiments, the invention provides a method of altering expression of a target nucleic acid in a eukaryotic cell comprising: contacting the cell with a Cas9 described herein, and an RNA guide or a nucleic acid encoding the RNA guide, wherein the RNA guide comprises a direct repeat sequence and a spacer sequence capable of hybridizing to the target nucleic acid, and wherein the Cas9 protein is capable of binding to the RNA guide and of causing a break in the target nucleic acid sequence complementary to the RNA guide.

In some embodiments, the invention provides a method of altering expression of a target nucleic acid in a eukaryotic cell comprising: contacting the cell with a Cas9 described herein, and an RNA guide or a nucleic acid encoding the RNA guide, wherein the RNA guide comprises a direct repeat sequence and a spacer sequence capable of hybridizing to the target nucleic acid, and wherein the Cas9 protein is capable of binding to the RNA guide and editing the target nucleic acid sequence complementary to the RNA guide.

In some embodiments, the invention provides a method of modifying a target nucleic acid in a eukaryotic cell comprising: contacting the cell with a Cas9 described herein, and an RNA guide or a nucleic acid encoding the RNA guide, wherein the RNA guide comprises a direct repeat sequence and a spacer sequence capable of hybridizing to the target nucleic acid, and wherein the Cas9 protein is capable of binding to the RNA guide and editing the target nucleic acid sequence complementary to the RNA guide.

Accordingly, in some embodiments, the Cas protein has about 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 1. In some embodiments, the Cas protein is identical to SEQ ID NO: 1.

Suitable guide RNA, Cas9 mutations and fusion proteins for use in the CRISPR-Cas9 system and method are as described throughout this disclosure.

In one aspect, the method comprises binding of the CRISPR-Cas9 to a target nucleic acid and effecting cleavage of a target nucleic acids. In some embodiments, the CRISPR-Cas9 system cleaves target DNA or RNA duplexes by introducing double-stranded breaks. In some embodiments, the CRISPR-Cas9 system cleaves target DNA or RNA by introducing single-stranded breaks or nicks.

In some embodiments, the CRISPR-Cas9 method or system comprises a fusion protein with an effector that modifies target DNA in a site-specific manner, where the modifying activity includes methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, demyristoylation activity, integrase activity, transposase activity, recombinase activity, polymerase activity, ligase activity, helicase activity, or nuclease activity, any of which can modify DNA or a DNA-associated polypeptide (e.g., a histone or DNA binding protein).

In some embodiments, the CRISPR-Cas9 method or system comprises a fusion protein with enzymes that can edit DNA sequences by chemically modifying nucleotide bases, including deaminase enzymes that can modify adenosine or cytosine bases and function as site-specific base editors. For example, APOBEC1 cytidine deaminase, which usually uses RNA as a substrate, can be targeted to single-stranded and double-stranded DNA when it is fused to Cas9, converting cytidine to uridine directly, and ADAR enzymes deaminate adenosine to inosine. Thus, ‘base editing’ using deaminases enables programmable conversion of one target DNA base into another. Various base editors are known in the art and can be used in the method and systems described herein. Exemplary base editors are described in, for example, Rees and Liu Nature Review Genetics, 2018, 19(12): 770-788, the contents of which are incorporated herein. Accordingly, in some embodiments, the Lachnospira UBA3212 Cas9 (LubCas9) described herein is a component of a nucleobase editor. In some embodiments, the base editor is the adenine deaminase TadA8 or TadA9.

In some embodiments, base editing results in the introduction of stop codons to silence genes. In some embodiments, base editing results in altered protein function by altering amino acid sequences.

In some embodiments, the CRISPR-Cas9 method or system comprises epigenetic modification of target DNA by fusion with a histone. In some embodiments, the CRISPR-Cas9 system comprises epigenetic modification of target DNA by fusion with an epigenetic modifying enzyme such as a reader, writer or eraser protein. In some embodiments, the CRISPR-Cas9 system comprises fusion with a histone modifying enzyme to alter the histone modification pattern in a selected region of target DNA. Histone modifications can occur in many different ways including methylation, acetylation, ubiquitination, phosphorylation, and in many different combinations, leading to structural changes in DNA. In some embodiments, histone modification leads to transcriptional repression or activation.

In some embodiments, the CRISPR-Cas9 method or system modulates transcription of target DNA by increasing or decreasing transcription through fusion with transcriptional activator proteins or transcriptional repressor proteins, small molecule/drug-responsive ytamscriptional regulators, inducible transcription regulators. In some embodiments, the CRISPR-Cas9 system is used to control the expression of a target coding mRNA (i.e. a protein encoding gene) where binding results in increased or decreased gene expression.

In some embodiments, the CRISPR-Cas9 method or system is used to control gene regulation by editing genetic regulatory elements such as promoters or enhancers.

In some embodiments, the CRISPR-Cas9 method or system is used to control the expression of a target non-coding RNA, including tRNA, rRNA, snoRNA, siRNA, miRNA, and long ncRNA.

In some embodiments, the CRISPR-Cas9 method or system is used for targeted engineering of chromatin loop structures. Targeted engineering of chromatin loops between regulatory genomic regions provides a means to manipulate endogenous chromatin structures and enable the formation of new enhancer-promoter connections to overcome genetic deficiencies or inhibit aberrant enhancer-promoter connections.

In some embodiments, CRISPR-Cas9 is used for live cell imaging. Fluorescently labelled Cas9 is targeted to repetitive genomic regions such as centromeres and telomeres to track native chromatin loci throughout the cell cycle and determine differential positioning of transcriptionally active and inactive regions in the 3D nuclear space.

In some embodiments, the CRISPR-Cas9 method or system is used for correction of pathogenic mutations by insertion of beneficial clinical variants or suppressor mutations.

### Nucleobase Editors

Disclosed herein, are novel base editors or nucleobase editors for editing, modifying or altering a target nucleotide sequence of a polynucleotide comprising a Lachnospira UBA3212 Cas9 (LubCas9). Described herein is a nucleobase editor or a base editor comprising a polynucleotide programmable nucleotide binding domain (e.g., LubCas9) and a nucleobase editing domain (e.g., adenosine deaminase). A polynucleotide programmable nucleotide binding domain (e.g., LubCas9), when in conjunction with a bound guide polynucleotide (e.g., gRNA), can specifically bind to a target polynucleotide sequence (i.e., via complementary base pairing between bases of the bound guide nucleic acid and bases of the target polynucleotide sequence) and thereby localize the base editor to the target nucleic acid sequence desired to be edited. In some embodiments, the target polynucleotide sequence comprises single-stranded DNA or double-stranded DNA. In some embodiments, the target polynucleotide sequence comprises RNA. In some embodiments, the target polynucleotide sequence comprises a DNA-RNA hybrid. As most of the known genetic variations associated with human disease are point mutations, methods that can more efficiently and cleanly make precise point mutations are needed. Base editing systems as provided herein provide a new way to provide genome editing without generating double-strand DNA breaks, without requiring a donor DNA template, and without inducing an excess of stochastic insertions and deletions.

The base editors provided herein are capable of modifying a specific nucleotide base without generating a significant proportion of indels. The term “indel(s)”, as used herein, refers to the insertion or deletion of a nucleotide base within a nucleic acid. Such insertions or deletions can lead to frame shift mutations within a coding region of a gene. In some embodiments, it is desirable to generate base editors that efficiently modify (e.g., mutate or deaminate) a specific nucleotide within a nucleic acid, without generating a large number of insertions or deletions (i.e., indels) in the target nucleotide sequence. In certain embodiments, any of the base editors provided herein are capable of generating a greater proportion of intended modifications (e.g., point mutations or deaminations) versus indels.

In some embodiments, any of base editor systems provided herein result in less than 50%, less than 40%0, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% indel formation in the target polynucleotide sequence.

Some aspects of the disclosure are based on the recognition that any of the base editors provided herein are capable of efficiently generating an intended mutation, such as a point mutation, in a nucleic acid (e.g., a nucleic acid within a genome of a subject) without generating a significant number of unintended mutations, such as unintended point mutations. In some embodiments, any of the base editors provided herein are capable of generating at least 0.01% of intended mutations (i.e. at least 0.01% base editing efficiency). In some embodiments, any of the base editors provided herein are capable of generating at least 0.01%, 1%, 2%, 3%, 4%, 5%, 10%, 15%, 20%, 25%, 30%, 40%, 45%, 50%, 60%, 70%, 80%, 90%, 95%, or 99% of intended mutations.

In some embodiments, the base editors provided herein are capable of generating a ratio of intended point mutations to indels that is greater than 1:1. In some embodiments, the base editors provided herein are capable of generating a ratio of intended point mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 8.5:1, at least 9:1, at least 10:1, at least 11:1, at least 12:1, at least 13:1, at least 14:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more.

The number of intended mutations and indels can be determined using any suitable method, for example, as described in International PCT Application Nos. PCT/2017/045381 (WO2018/027078) and PCT/US2016/058344 (WO2017/070632); Komor, A. C., et al., “Programmable editing of a target base in genomic DNA without double-stranded DNA cleavage” Nature 533, 420-424 (2016); Gaudelli, N. M., et al., “Programmable base editing of A⋅T to G⋅C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017); the entire contents of which are hereby incorporated by reference.

In some embodiments, to calculate indel frequencies, sequencing reads are scanned for exact matches to two 10-bp sequences that flank both sides of a window in which indels can occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively. In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor.

The number of indels formed at a target nucleotide region can depend on the amount of time a nucleic acid (e.g., a nucleic acid within the genome of a cell) is exposed to a base editor. In some embodiments, the number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing the target nucleotide sequence (e.g., a nucleic acid within the genome of a cell) to a base editor. It should be appreciated that the characteristics of the base editors as described herein can be applied to any of the fusion proteins, or methods of using the fusion proteins provided herein.

### Therapeutic Applications

The CRISPR-Cas9 methods or systems described herein can have various therapeutic applications. Accordingly, in some embodiments, a method of treating a disorder or a disease in a subject in need thereof is provided, the method comprising administering to the subject a CRISPR-Cas9 system comprising a Cas9 as described herein, wherein the guide RNA is complementary to at least 10 nucleotides of a target nucleic acid associated with the condition or disease; wherein the Cas protein associates with the guide RNA; wherein the guide RNA binds to the target nucleic acid; wherein the Cas protein causes a break in the target nucleic acid, optionally wherein the Cas9 is an inactive Cas9 (dCas9) fused to a deaminase and results in one or more base edits in the target nucleic acid, thereby treating the disorder or disease.

In some embodiments, the CRISPR-Cas9 methods or systems can be used to treat various diseases and disorders, e.g., genetic disorders (e.g., monogenetic diseases), diseases that can be treated by nuclease activity, and various cancers, etc.

In some embodiments, the CRISPR methods or systems described herein can be used to edit a target nucleic acid to modify the target nucleic acid (e.g., by inserting, deleting, or mutating one or more nucleic acid residues). For example, in some embodiments the CRISPR systems described herein comprise an exogenous donor template nucleic acid (e.g., a DNA molecule or a RNA molecule), which comprises a desirable nucleic acid sequence. Upon resolution of a cleavage event induced with the CRISPR system described herein, the molecular machinery of the cell will utilize the exogenous donor template nucleic acid in repairing and/or resolving the cleavage event. Alternatively, the molecular machinery of the cell can utilize an endogenous template in repairing and/or resolving the cleavage event. In some embodiments, the CRISPR systems described herein may be used to alter a target nucleic acid resulting in an insertion, a deletion, and/or a point mutation). In some embodiments, the insertion is a scarless insertion (i.e., the insertion of an intended nucleic acid sequence into a target nucleic acid resulting in no additional unintended nucleic acid sequence upon resolution of the cleavage event). Donor template nucleic acids may be double stranded or single stranded nucleic acid molecules (e.g., DNA or RNA). In some embodiments, the CRISPR methods or systems described herein comprise a nucleobase editor. For example, in some embodiments, the Lachnospira UBA3212 Cas9 (LubCas9) described herein is fused to a polypeptide having nucleobase editing activity.

In one aspect, the CRISPR methods or systems described herein can be used for treating a disease caused by overexpression of RNAs, toxic RNAs, and/or mutated RNAs (e.g., splicing defects or truncations).

In some embodiments, the CRISPR methods or systems described herein can also target trans-acting mutations affecting RNA-dependent functions that cause various diseases.

In some embodiments, the CRISPR methods or systems described herein can also be used to target mutations disrupting the cis-acting splicing codes that can cause splicing defects and diseases.

The CRISPR methods or systems described herein can further be used for antiviral activity, in particular against RNA viruses. The CRISPR-associated proteins can target the viral RNAs using suitable RNA guides selected to target viral RNA sequences.

The CRISPR methods or systems described herein can also be used to treat a cancer in a subject (e.g., a human subject). For example, the CRISPR-associated proteins described herein can be programmed with crRNA targeting a RNA molecule that is aberrant (e.g., comprises a point mutation or are alternatively-spliced) and found in cancer cells to induce cell death in the cancer cells (e.g., via apoptosis).

Further, the CRISPR methods or systems described herein can also be used to treat an infectious disease in a subject. For example, the CRISPR-associated proteins described herein can be programmed with crRNA targeting a RNA molecule expressed by an infectious agent (e.g., a bacteria, a virus, a parasite or a protozoan) in order to target and induce cell death in the infectious agent cell. The CRISPR systems may also be used to treat diseases where an intracellular infectious agent infects the cells of a host subject. By programming the CRISPR-associated protein to target a RNA molecule encoded by an infectious agent gene, cells infected with the infectious agent can be targeted and cell death induced.

Furthermore, in vitro RNA sensing assays can be used to detect specific RNA substrates. The CRISPR-associated proteins can be used for RNA-based sensing in living cells. Examples of applications are diagnostics by sensing of, for examples, disease-specific RNAs.

In applications in which it is desirable to insert a polynucleotide sequence into a target DNA sequence, a polynucleotide comprising a donor sequence to be inserted is also provided to the cell. By a “donor sequence” or “donor polynucleotide” it is meant a nucleic acid sequence to be inserted at the cleavage site induced by a site-directed modifying polypeptide. The donor polynucleotide will contain sufficient homology to a genomic sequence at the cleavage site, e.g. 70%, 80%, 85%, 90%, 95%, or 100% homology with the nucleotide sequences flanking the cleavage site, e.g. within about 50 bases or less of the cleavage site, e.g. within about 30 bases, within about 15 bases, within about 10 bases, within about 5 bases, or immediately flanking the cleavage site, to support homology-directed repair between it and the genomic sequence to which it bears homology. Approximately 25, 50, 100, or 200 nucleotides, or more than 200 nucleotides, of sequence homology between a donor and a genomic sequence (or any integral value between 10 and 200 nucleotides, or more) will support homology-directed repair. Donor sequences can be of any length, e.g. 10 nucleotides or more, 50 nucleotides or more, 100 nucleotides or more, 250 nucleotides or more, 500 nucleotides or more, 1000 nucleotides or more, 5000 nucleotides or more, etc.

The donor sequence is typically not identical to the genomic sequence that it replaces. Rather, the donor sequence may contain at least one or more single base changes, insertions, deletions, inversions or rearrangements with respect to the genomic sequence, so long as sufficient homology is present to support homology-directed repair. In some embodiments, the donor sequence comprises a non-homologous sequence flanked by two regions of homology, such that homology-directed repair between the target DNA region and the two flanking sequences results in insertion of the non-homologous sequence at the target region. Donor sequences may also comprise a vector backbone containing sequences that are not homologous to the DNA region of interest and that are not intended for insertion into the DNA region of interest. Generally, the homologous region(s) of a donor sequence will have at least 50% sequence identity to a genomic sequence with which recombination is desired. In certain embodiments, 60%, 70%, 80%, 90%, 95%, 98%, 99%, or 99.9% sequence identity is present. Any value between 1% and 100% sequence identity can be present, depending upon the length of the donor polynucleotide.

The donor sequence may comprise certain sequence differences as compared to the genomic sequence, e.g. restriction sites, nucleotide polymorphisms, selectable markers (e.g., drug resistance genes, fluorescent proteins, enzymes etc.), etc., which may be used to assess for successful insertion of the donor sequence at the cleavage site or in some cases may be used for other purposes (e.g., to signify expression at the targeted genomic locus). In some cases, if located in a coding region, such nucleotide sequence differences will not change the amino acid sequence, or will make silent amino acid changes (i.e., changes which do not affect the structure or function of the protein). Alternatively, these sequences differences may include flanking recombination sequences such as FLPs, loxP sequences, or the like, that can be activated at a later time for removal of the marker sequence.

The donor sequence may be provided to the cell as single-stranded DNA, single-stranded RNA, double-stranded DNA, or double-stranded RNA. It may be introduced into a cell in linear or circular form. If introduced in linear form, the ends of the donor sequence may be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. Additional methods for protecting exogenous polynucleotides from degradation include, but are not limited to, addition of terminal amino group(s) and the use of modified internucleotide linkages such as, for example, phosphorothioates, phosphor amidates, and O-methyl ribose or deoxyribose residues. As an alternative to protecting the termini of a linear donor sequence, additional lengths of sequence may be included outside of the regions of homology that can be degraded without impacting recombination. A donor sequence can be introduced into a cell as part of a vector molecule having additional sequences such as, for example, replication origins, promoters and genes encoding antibiotic resistance. Moreover, donor sequences can be introduced as naked nucleic acid, as nucleic acid complexed with an agent such as a liposome or poloxamer, or can be delivered by viruses (e.g., adenovirus, AAV), as described above for nucleic acids encoding a DNA-targeting RNA and/or site-directed modifying polypeptide and/or donor polynucleotide.

Following the methods described above, a DNA region of interest may be cleaved and modified, i.e. “genetically modified”, ex vivo. In some embodiments, as when a selectable marker has been inserted into the DNA region of interest, the population of cells may be enriched for those comprising the genetic modification by separating the genetically modified cells from the remaining population. Prior to enriching, the “genetically modified” cells may make up only about 1% or more (e.g., 2% or more, 3% or more, 4% or more, 5% or more, 6% or more, 7% or more, 8% or more, 9% or more, 10% or more, 15% or more, or 20% or more) of the cellular population. Separation of “genetically modified” cells may be achieved by any convenient separation technique appropriate for the selectable marker used. For example, if a fluorescent marker has been inserted, cells may be separated by fluorescence activated cell sorting, whereas if a cell surface marker has been inserted, cells may be separated from the heterogeneous population by affinity separation techniques, e.g. magnetic separation, affinity chromatography, “panning” with an affinity reagent attached to a solid matrix, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide). Any technique may be employed which is not unduly detrimental to the viability of the genetically modified cells. Cell compositions that are highly enriched for cells comprising modified DNA are achieved in this manner. By “highly enriched”, it is meant that the genetically modified cells will be 70% or more, 75% or more, 80% or more, 85% or more, 90% or more of the cell composition, for example, about 95% or more, or 98% or more of the cell composition. In other words, the composition may be a substantially pure composition of genetically modified cells.

Genetically modified cells produced by the methods described herein may be used immediately. Alternatively, the cells may be frozen at liquid nitrogen temperatures and stored for long periods of time, being thawed and capable of being reused. In such cases, the cells will usually be frozen in 10% dimethylsulfoxide (DMSO), 50% serum, 40% buffered medium, or some other such solution as is commonly used in the art to preserve cells at such freezing temperatures, and thawed in a manner as commonly known in the art for thawing frozen cultured cells.

The genetically modified cells may be cultured in vitro under various culture conditions. The cells may be expanded in culture. i.e. grown under conditions that promote their proliferation. Culture medium may be liquid or semi-solid, e.g. containing agar, methylcellulose, etc. The cell population may be suspended in an appropriate nutrient medium, such as Iscove's modified DMEM or RPMI 1640, normally supplemented with fetal calf serum (about 5-10%),

L-glutamine, a thiol, particularly 2-mercaptoethanol, and antibiotics, e.g. penicillin and streptomycin. The culture may contain growth factors to which the regulatory T cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

Cells that have been genetically modified in this way may be transplanted to a subject for purposes such as gene therapy, e.g. to treat a disease or as an antiviral, antipathogenic, or anticancer therapeutic, for the production of genetically modified organisms in agriculture, or for biological research. The subject may be a neonate, a juvenile, or an adult. Of particular interest are mammalian subjects. Mammalian species that may be treated with the present methods include canines and felines; equines; bovines; ovines; etc. and primates, particularly humans. Animal models, particularly small mammals (e.g. mouse, rat, guinea pig, hamster, lagomorpha (e.g., rabbit), etc.) may be used for experimental investigations.

Cells may be provided to the subject alone or with a suitable substrate or matrix, e.g. to support their growth and/or organization in the tissue to which they are being transplanted. Usually, at least 1×10³ cells will be administered, for example 5×10³ cells, 1×10⁴ cells, 5×10⁴ cells, 1×10⁵ cells, 1×10⁶ cells or more. The cells may be introduced to the subject via any of the following routes: parenteral, subcutaneous, intravenous, intracranial, intraspinal, intraocular, or into spinal fluid. The cells may be introduced by injection, catheter, or the like. Cells may also be introduced into an embryo (e.g., a blastocyst) for the purpose of generating a transgenic animal (e.g., a transgenic mouse).

The number of administrations of treatment to a subject may vary. Introducing the genetically modified cells into the subject may be a one-time event; but in certain situations, such treatment may elicit improvement for a limited period of time and require an on-going series of repeated treatments. In other situations, multiple administrations of the genetically modified cells may be required before an effect is observed. The exact protocols depend upon the disease or condition, the stage of the disease and parameters of the individual subject being treated.

In other aspects of the invention, the DNA-targeting RNA and/or site-directed modifying polypeptide and/or donor polynucleotide are employed to modify cellular DNA in vivo, again for purposes such as gene therapy, e.g. to treat a disease or as an antiviral, antipathogenic, or anticancer therapeutic, for the production of genetically modified organisms in agriculture, or for biological research. In these in vivo embodiments, a DNA-targeting RNA and/or site-directed modifying polypeptide and/or donor polynucleotide are administered directly to the individual. A DNA-targeting RNA and/or site-directed modifying polypeptide and/or donor polynucleotide may be administered by any of a number of well-known methods in the art for the administration of peptides, small molecules and nucleic acids to a subject. A DNA-targeting RNA and/or site-directed modifying polypeptide and/or donor polynucleotide can be incorporated into a variety of formulations. More particularly, a DNA-targeting RNA and/or site-directed modifying polypeptide and/or donor polynucleotide of the present invention can be formulated into pharmaceutical compositions by combination with appropriate pharmaceutically acceptable carriers or diluents.

Pharmaceutical preparations are compositions that include one or more a DNA-targeting RNA and/or site-directed modifying polypeptide and/or donor polynucleotide present in a pharmaceutically acceptable vehicle. “Pharmaceutically acceptable vehicles” may be vehicles approved by a regulatory agency of the Federal or a state government or listed in the U.S.

Pharmacopeia or other generally recognized pharmacopeia for use in mammals, such as humans. The term “vehicle” refers to a diluent, adjuvant, excipient, or carrier with which a compound of the invention is formulated for administration to a mammal. Such pharmaceutical vehicles can be lipids, e.g. liposomes, e.g. liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semisolid, liquid or gaseous forms, such as tablets, capsules, powders, granules, ointments, solutions, suppositories, injections, inhalants, gels, microspheres, and aerosols. As such, administration of the a DNA-targeting RNA and/or site-directed modifying polypeptide and/or donor polynucleotide can be achieved in various ways, including oral, buccal, rectal, parenteral, intraperitoneal, intradermal, transdermal, intratracheal, intraocular, etc., administration. The active agent may be systemic after administration or may be localized by the use of regional administration, intramural administration, or use of an implant that acts to retain the active dose at the site of implantation. The active agent may be formulated for immediate activity or it may be formulated for sustained release.

For some conditions, particularly central nervous system conditions, it may be necessary to formulate agents to cross the blood-brain barrier (BBB). One strategy for drug delivery through the blood-brain barrier (BBB) entails disruption of the BBB, either by osmotic means such as mannitol or leukotrienes, or biochemically by the use of vasoactive substances such as bradykinin. The potential for using BBB opening to target specific agents to brain tumors is also an option. A BBB disrupting agent can be co-administered with the therapeutic compositions of the invention when the compositions are administered by intravascular injection. Other strategies to go through the BBB may entail the use of endogenous transport systems, including Caveolin-1 mediated transcytosis, carrier-mediated transporters such as glucose and amino acid carriers, receptor-mediated transcytosis for insulin or transferrin, and active efflux transporters such as p-glycoprotein. Active transport moieties may also be conjugated to the therapeutic compounds for use in the invention to facilitate transport across the endothelial wall of the blood vessel.

Alternatively, drug delivery of therapeutics agents behind the BBB may be by local delivery, for example by intrathecal delivery.

Typically, an effective amount of a DNA-targeting RNA and/or site-directed modifying polypeptide and/or donor polynucleotide are provided. As discussed above with regard to ex vivo methods, an effective amount or effective dose of a DNA-targeting RNA and/or site-directed modifying polypeptide and/or donor polynucleotide in vivo is the amount to induce a 2 fold increase or more in the amount of recombination observed between two homologous sequences relative to a negative control, e.g. a cell contacted with an empty vector or irrelevant polypeptide. The amount of recombination may be measured by any convenient method, e.g. as described above and known in the art. The calculation of the effective amount or effective dose of a DNA-targeting RNA and/or site-directed modifying polypeptide and/or donor polynucleotide to be administered is within the skill of one of ordinary skill in the art, and will be routine to those persons skilled in the art. The final amount to be administered will be dependent upon the route of administration and upon the nature of the disorder or condition that is to be treated.

The effective amount given to a particular patient will depend on a variety of factors, several of which will differ from patient to patient. A competent clinician will be able to determine an effective amount of a therapeutic agent to administer to a patient to halt or reverse the progression the disease condition as required. Utilizing LD50 animal data, and other information available for the agent, a clinician can determine the maximum safe dose for an individual, depending on the route of administration. For instance, an intravenously administered dose may be more than an intrathecally administered dose, given the greater body of fluid into which the therapeutic composition is being administered. Similarly, compositions which are rapidly cleared from the body may be administered at higher doses, or in repeated doses, in order to maintain a therapeutic concentration. Utilizing ordinary skill, the competent clinician will be able to optimize the dosage of a particular therapeutic in the course of routine clinical trials.

For inclusion in a medicament, a DNA-targeting RNA and/or site-directed modifying polypeptide and/or donor polynucleotide may be obtained from a suitable commercial source. As a general proposition, the total pharmaceutically effective amount of the a DNA-targeting RNA and/or site-directed modifying polypeptide and/or donor polynucleotide administered parenterally per dose will be in a range that can be measured by a dose response curve.

Therapies based on a DNA-targeting RNA and/or site-directed modifying polypeptide and/or donor polynucleotides, i.e. preparations of a DNA-targeting RNA and/or site-directed modifying polypeptide and/or donor polynucleotide to be used for therapeutic administration, must be sterile. Sterility is readily accomplished by filtration through sterile filtration membranes (e.g., 0.2μηι membranes). Therapeutic compositions generally are placed into a container having a sterile access port, for example, an intravenous solution bag or vial having a stopper pierceable by a hypodermic injection needle. The therapies based on a DNA-targeting RNA and/or site-directed modifying polypeptide and/or donor polynucleotide may be stored in unit or multi-dose containers, for example, sealed ampules or vials, as an aqueous solution or as a lyophilized formulation for reconstitution. As an example of a lyophilized formulation, 10-mL vials are filled with 5 ml of sterile-filtered 1% (w/v) aqueous solution of compound, and the resulting mixture is lyophilized. The infusion solution is prepared by reconstituting the lyophilized compound using bacteriostatic Water-for-Injection.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, and enhance solubility or uptake). Examples of such modifications or complexing agents include sulfate, gluconate, citrate and phosphate. The nucleic acids or polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Therapies that exhibit large therapeutic indices are preferred.

The data obtained from cell culture and/or animal studies can be used in formulating a range of dosages for humans. The dosage of the active ingredient typically lines within a range of circulating concentrations that include the ED50 with low toxicity. The dosage can vary within this range depending upon the dosage form employed and the route of administration utilized.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

### Delivery Systems

The CRISPR systems described herein, or components thereof, nucleic acid molecules thereof, and/or nucleic acid molecules encoding or providing components thereof, CRISPR-associated proteins, or RNA guides, can be delivered by various delivery systems such as vectors, e.g., plasmids and delivery vectors. Exemplary embodiments are described below. The CRISPR systems (e.g., including the Cas9 comprising nucleobase editor described herein) can be encoded on a nucleic acid that is contained in a viral vector. Viral vectors can include lentivirus, Adenovirus, Retrovirus, and Adeno-associated viruses (AAVs). Viral vectors can be selected based on the application. For example, AAVs are commonly used for gene delivery in vivo due to their mild immunogenicity. Adenoviruses are commonly used as vaccines because of the strong immunogenic response they induce. Packaging capacity of the viral vectors can limit the size of the base editor that can be packaged into the vector. For example, the packaging capacity of the AAVs is ˜4.5 kb including two 145 base inverted terminal repeats (ITRs).

AAV is a small, single-stranded DNA dependent virus belonging to the parvovirus family. The 4.7 kb wild-type (wt) AAV genome is made up of two genes that encode four replication proteins and three capsid proteins, respectively, and is flanked on either side by 145-bp inverted terminal repeats (ITRs). The virion is composed of three capsid proteins, Vp1, Vp2, and Vp3, produced in a 1:1:10 ratio from the same open reading frame but from differential splicing (Vp1) and alternative translational start sites (Vp2 and Vp3, respectively). Vp3 is the most abundant subunit in the virion and participates in receptor recognition at the cell surface defining the tropism of the virus. A phospholipase domain, which functions in viral infectivity, has been identified in the unique N terminus of Vp1.

Similar to wt AAV, recombinant AAV (rAAV) utilizes the cis-acting 145-bp ITRs to flank vector transgene cassettes, providing up to 4.5 kb for packaging of foreign DNA. Subsequent to infection, rAAV can express a fusion protein of the invention and persist without integration into the host genome by existing episomally in circular head-to-tail concatemers. Although there are numerous examples of rAAV success using this system, in vitro and in vivo, the limited packaging capacity has limited the use of AAV-mediated gene delivery when the length of the coding sequence of the gene is equal or greater in size than the wt AAV genome.

The small packaging capacity of AAV vectors makes the delivery of a number of genes that exceed this size and/or the use of large physiological regulatory elements challenging. These challenges can be addressed, for example, by dividing the protein(s) to be delivered into two or more fragments, wherein the N-terminal fragment is fused to a split intein-N and the C-terminal fragment is fused to a split intein-C. These fragments are then packaged into two or more AAV vectors. As used herein, “intein” refers to a self-splicing protein intron (e.g., peptide) that ligates flanking N-terminal and C-terminal exteins (e.g., fragments to be joined). The use of certain inteins for joining heterologous protein fragments is described, for example, in Wood et al., J. Biol. Chem. 289(21); 14512-9 (2014). For example, when fused to separate protein fragments, the inteins IntN and IntC recognize each other, splice themselves out and simultaneously ligate the flanking N- and C-terminal exteins of the protein fragments to which they were fused, thereby reconstituting a full-length protein from the two protein fragments. Other suitable inteins will be apparent to a person of skill in the art.

In some embodiments, the CRISPR system of the invention can vary in length. In some embodiments, a protein fragment ranges from 2 amino acids to about 1000 amino acids in length. In some embodiments, a protein fragment ranges from about 5 amino acids to about 500 amino acids in length. In some embodiments, a protein fragment ranges from about 20 amino acids to about 200 amino acids in length. In some embodiments, a protein fragment ranges from about 10 amino acids to about 100 amino acids in length. Suitable protein fragments of other lengths will be apparent to a person of skill in the art.

In some embodiments, a portion or fragment of a nuclease (e.g., Cas9) is fused to an intein. The nuclease can be fused to the N-terminus or the C-terminus of the intein. In some embodiments, a portion or fragment of a fusion protein is fused to an intein and fused to an AAV capsid protein. The intein, nuclease and capsid protein can be fused together in any arrangement (e.g., nuclease-intein-capsid, intein-nuclease-capsid, capsid-intein-nuclease, etc.). In some embodiments, the N-terminus of an intein is fused to the C-terminus of a fusion protein and the C-terminus of the intein is fused to the N-terminus of an AAV capsid protein.

In one embodiment, dual AAV vectors are generated by splitting a large transgene expression cassette in two separate halves (5′ and 3′ ends, or head and tail), where each half of the cassette is packaged in a single AAV vector (of <5 kb). The re-assembly of the full-length transgene expression cassette is then achieved upon co-infection of the same cell by both dual AAV vectors followed by: (1) homologous recombination (HR) between 5′ and 3′ genomes (dual AAV overlapping vectors); (2) ITR-mediated tail-to-head concatemerization of 5′ and 3′ genomes (dual AAV trans-splicing vectors); or (3) a combination of these two mechanisms (dual AAV hybrid vectors). The use of dual AAV vectors in vivo results in the expression of full-length proteins. The use of the dual AAV vector platform represents an efficient and viable gene transfer strategy for transgenes of >4.7 kb in size.

The disclosed strategies for designing CRISPR systems including the Cas9 described herein can be useful for generating CRISPR systems capable of being packaged into a viral vector. The use of RNA or DNA viral based systems for the delivery of a base editor takes advantage of highly evolved processes for targeting a virus to specific cells in culture or in the host and trafficking the viral payload to the nucleus or host cell genome. Viral vectors can be administered directly to cells in culture, patients (in vivo), or they can be used to treat cells in vitro, and the modified cells can optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SiV), human immuno deficiency virus (HIV), and combinations thereof (See, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700).

Retroviral vectors, especially lentiviral vectors, can require polynucleotide sequences smaller than a given length for efficient integration into a target cell. For example, retroviral vectors of length greater than 9 kb can result in low viral titers compared with those of smaller size. In some aspects, a CRISPR system (e.g., including the Cas9 disclosed herein) of the present disclosure is of sufficient size so as to enable efficient packaging and delivery into a target cell via a retroviral vector. In some cases, a Cas9 is of a size so as to allow efficient packing and delivery even when expressed together with a guide nucleic acid and/or other components of a targetable nuclease system.

In applications where transient expression is preferred, adenoviral based systems can be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors can also be used to transduce cells with target nucleic acids, e.g., in the in vitro production of nucleic acids and peptides, and for in vivo and ex vivo gene therapy procedures (See. e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). The construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

A CRISPR system (e.g., including the Cas9 disclosed herein) described herein can therefore be delivered with viral vectors. One or more components of the base editor system can be encoded on one or more viral vectors. For example, a base editor and guide nucleic acid can be encoded on a single viral vector. In other cases, the base editor and guide nucleic acid are encoded on different viral vectors. In either case, the base editor and guide nucleic acid can each be operably linked to a promoter and terminator.

The combination of components encoded on a viral vector can be determined by the cargo size constraints of the chosen viral vector.

### Non-Viral Delivery of Base Editors

Non-viral delivery approaches for CRISPR are also available. One important category of non-viral nucleic acid vectors are nanoparticles, which can be organic or inorganic. Nanoparticles are well known in the art. Any suitable nanoparticle design can be used to deliver genome editing system components or nucleic acids encoding such components. For instance, organic (e.g. lipid and/or polymer) nanoparticles can be suitable for use as delivery vehicles in certain embodiments of this disclosure. Exemplary lipids for use in nanoparticle formulations, and/or gene transfer are shown in Table 5 (below).

TABLE 5
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N-[1-(2,3-Dioleyloxy)prophyl]N,N,N-trimethylammonium | DOTMA | Cationic |
| chloride | | |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)-2,4,6-trimethylpyridinium | 2Oc | Cationic |
| 2,3-Dioleyloxy-N-[2(sperminecarboxamido-ethyl]-N,N- | DOSPA | Cationic |
| dimethyl-1-propanaminium trifluoroacetate | | |
| 1,2-Dioleyl-3-trimethylammonium-propane | DOPA | Cationic |
| N-(2-Hydroxyethyl)-N,N-dimethyl-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-(N′,N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyl- | CLIP-6 | Cationic |
| oxymethyloxy)ethyl]trimethylammoniun bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylpho sphocholine | DSEPC | Cationic |
| N-Palmitoyl D-erythro-sphingosyl carbamoyl-spermine | CCS | Cationic |
| N-t-Butyl-N0-tetradecyl-3-tetradecylaminopropionamidine | diC14-amidine | Cationic |
| Octadecenolyoxy[ethyl-2-heptadecenyl-3 hydroxyethyl] | DOTIM | Cationic |
| imidazolinium chloride | | |
| N1-Cholesteryloxycarbonyl-3,7-diazanonane-1,9-diamine | CDAN | Cationic |
| 2-(3-[Bis(3-amino-propyl)-amino]propylamino)-N- | RPR209120 | Cationic |
| ditetradecylcarbamoylme-ethyl-acetamide | | |
| 1,2-dilinoleyloxy-3-dimethylaminopropane | DLinDMA | Cationic |
| 2,2-dilinoleyl-4-dimethylaminoethyl-[1,3]-dioxolane | DLin-KC2-DMA | Cationic |
| dilinoleyl-methyl-4-dimethylaminobutyrate | DLin-MC3-DMA | Cationic |

Table 6 lists exemplary polymers for use in gene transfer and/or nanoparticle formulations.

TABLE 6
Polymers Used for Gene Transfer

| Polymer | Abbreviation |
|---|---|
| Poly(ethylene)glycol | PEG |
| Polyethylenimine | PEI |
| Dithiobis (succinimidylpropionate) | DSP |
| Dimethyl-3,3′-dithiobispropionimidate | DTBP |
| Poly(ethylene imine)biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amidoethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

Table 7 summarizes delivery methods for a polynucleotide encoding a Cas9 described herein.

TABLE 7

| | | Delivery into | | | Type of |
|---|---|---|---|---|---|
| | | Non-Dividing | Duration of | Genome | Molecule |
| Delivery | Vector/Mode | Cells | Expression | Integration | Delivered |
| Physical | (e.g., | YES | Transient | NO | Nucleic Acids |
| | electroporation, | | | | and Proteins |
| | particle gun, | | | | |
| | Calcium | | | | |
| | Phosphate | | | | |
| | transfection | | | | |
| Viral | Retrovirus | NO | Stable | YES | RNA |
| | Lentivirus | YES | Stable | YES/NO with | RNA |
| | | | | modification | |
| | Adenovirus | YES | Transient | NO | DNA |
| | Adeno- | YES | Stable | NO | DNA |
| | Associated | | | | |
| | Virus (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic Acids |
| | Liposomes | | | what is | and Proteins |
| | | | | delivered | |
| | Polymeric | YES | Transient | Depends on | Nucleic Acids |
| | Nanoparticles | | | what is | and Proteins |
| | | | | delivered | |
| Biological | Attenuated | YES | Transient | NO | Nucleic Acids |
| Non-Viral | Bacteria | | | | |
| Delivery | Engineered | YES | Transient | NO | Nucleic Acids |
| Vehicles | Bacteriophages | YES | Transient | NO | Nucleic Acids |
| | Mammalian | | | | |
| | Virus-like | | | | |
| | Particles | | | | |
| | Biological | YES | Transient | NO | Nucleic Acids |
| | liposomes: | | | | |
| | Erythrocyte | | | | |
| | Ghosts and | | | | |
| | Exosomes | | | | |

In another aspect, the delivery of genome editing system components or nucleic acids encoding such components, for example, a nucleic acid binding protein such as, for example, Cas9 or variants thereof, optionally fused to a polypeptide having biological activity (e.g., a nucleobase editor), and a gRNA targeting a genomic nucleic acid sequence of interest, may be accomplished by delivering a ribonucleoprotein (RNP) to cells. The RNP comprises the nucleic acid binding protein, e.g., Cas9, in complex with the targeting gRNA. RNPs may be delivered to cells using known methods, such as electroporation, nucleofection, or cationic lipid-mediated methods, for example, as reported by Zuris, J. A. et al., 2015, Nat. Biotechnology, 33(1):73-80. RNPs are advantageous for use in CRISPR base editing systems, particularly for cells that are difficult to transfect, such as primary cells. In addition. RNPs can also alleviate difficulties that may occur with protein expression in cells, especially when eukaryotic promoters, e.g., CMV or EF1A, which may be used in CRISPR plasmids, are not well-expressed. Advantageously, the use of RNPs does not require the delivery of foreign DNA into cells. Moreover, because an RNP comprising a nucleic acid binding protein and gRNA complex is degraded over time, the use of RNPs has the potential to limit off-target effects. In a manner similar to that for plasmid based techniques, RNPs can be used to deliver binding protein (e.g., Cas9 variants) and to direct homology directed repair (HDR).

A promoter used to drive the CRISPR system (e.g., including the Cas9 described herein) can include AAV ITR. This can be advantageous for eliminating the need for an additional promoter element, which can take up space in the vector. The additional space freed up can be used to drive the expression of additional elements, such as a guide nucleic acid or a selectable marker. ITR activity is relatively weak, so it can be used to reduce potential toxicity due to over expression of the chosen nuclease.

Any suitable promoter can be used to drive expression of the Cas9 and, where appropriate, the guide nucleic acid. For ubiquitous expression, promoters that can be used include CMV, CAG, CBh, PGK, SV40, Ferritin heavy or light chains, etc. For brain or other CNS cell expression, suitable promoters can include: SynapsinI for all neurons, CaMKIIalpha for excitatory neurons, GAD67 or GAD65 or VGAT for GABAergic neurons, etc. For liver cell expression, suitable promoters include the Albumin promoter. For lung cell expression, suitable promoters can include SP-B. For endothelial cells, suitable promoters can include ICAM. For hematopoietic cells suitable promoters can include IFNbeta or CD45. For Osteoblasts suitable promoters can include OG-2.

In some cases, a Cas9 of the present disclosure is of small enough size to allow separate promoters to drive expression of the base editor and a compatible guide nucleic acid within the same nucleic acid molecule. For instance, a vector or viral vector can comprise a first promoter operably linked to a nucleic acid encoding the base editor and a second promoter operably linked to the guide nucleic acid.

The promoter used to drive expression of a guide nucleic acid can include: Pol III promoters such as U6 or H1 Use of Pol II promoter and intronic cassettes to express gRNA Adeno Associated Virus (AAV).

A Cas9 described herein with or without one or more guide nucleic can be delivered using adeno associated virus (AAV), lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus. AAV and adenovirus. For example, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses can be based on or extrapolated to an average 70 kg individual (e.g. a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific base editing, the expression of the base editor and optional guide nucleic acid can be driven by a cell-type specific promoter.

For in vivo delivery, AAV can be advantageous over other viral vectors. In some cases, AAV allows low toxicity, which can be due to the purification method not requiring ultra-centrifugation of cell particles that can activate the immune response. In some cases, AAV allows low probability of causing insertional mutagenesis because it doesn't integrate into the host genome.

AAV has a packaging limit of 4.5 or 4.75 Kb. Constructs larger than 4.5 or 4.75 Kb can lead to significantly reduced virus production. For example. SpCas9 is quite large, the gene itself is over 4.1 Kb, which makes it difficult for packing into AAV. Therefore, embodiments of the present disclosure include utilizing a disclosed Cas9 which is shorter in length than conventional Cas9.

An AAV can be AAV1, AAV2, AAV5 or any combination thereof. One can select the type of AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. A tabulation of certain AAV serotypes as to these cells can be found in Grimm, D. et al, J. Virol. 82: 5887-5911 (2008)).

Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

Lentiviruses can be prepared as follows. After cloning pCasES10 (which contains a lentiviral transfer plasmid backbone), HEK293FT at low passage (p=5) were seeded in a T-75 flask to 50% confluence the day before transfection in DMEM with 10% fetal bovine serum and without antibiotics. After 20 hours, media is changed to OptiMEM (serum-free) media and transfection was done 4 hours later. Cells are transfected with 10 μg of lentiviral transfer plasmid (pCasES10) and the following packaging plasmids: 5 μg of pMD2.G (VSV-g pseudotype), and 7.5 μg of psPAX2 (gag/pol/rev/tat). Transfection can be done in 4 mL OptiMEM with a cationic lipid delivery agent (50 μl Lipofectamine 2000 and 100 ul Plus reagent). After 6 hours, the media is changed to antibiotic-free DMEM with 10% fetal bovine serum. These methods use serum during cell culture, but serum-free methods are preferred.

Lentivirus can be purified as follows. Viral supernatants are harvested after 48 hours. Supernatants are first cleared of debris and filtered through a 0.45 μm low protein binding (PVDF) filter. They are then spun in an ultracentrifuge for 2 hours at 24,000 rpm. Viral pellets are resuspended in 50 μl of DMEM overnight at 4° C. They are then aliquoted and immediately frozen at −80° C.

In another embodiment, minimal non-primate lentiviral vectors based on the equine infectious anemia virus (EIAV) are also contemplated. In another embodiment, RetinoStat®, an equine infectious anemia virus-based lentiviral gene therapy vector that expresses angiostatic proteins endostatin and angiostatin that is contemplated to be delivered via a subretinal injection. In another embodiment, use of self-inactivating lentiviral vectors is contemplated.

Any RNA of the systems, for example a guide RNA or a Cas9-encoding mRNA, can be delivered in the form of RNA. Cas9 encoding mRNA can be generated using in vitro transcription. For example, Cas9 mRNA can be synthesized using a PCR cassette containing the following elements: T7 promoter, optional kozak sequence (GCCACC), nuclease sequence, and 3′ UTR such as a 3′ UTR from beta globin-polyA tail. The cassette can be used for transcription by T7 polymerase. Guide polynucleotides (e.g., gRNA) can also be transcribed using in vitro transcription from a cassette containing a T7 promoter, followed by the sequence “G”, and guide polynucleotide sequence.

To enhance expression and reduce possible toxicity, the Cas9 sequence and/or the guide nucleic acid can be modified to include one or more modified nucleoside e.g. using pseudo-U or 5-Methyl-C.

The disclosure in some embodiments comprehends a method of modifying a cell or organism. The cell can be a prokaryotic cell or a eukaryotic cell. The cell can be a mammalian cell. The mammalian cell many be a non-human primate, bovine, porcine, rodent or mouse cell. The modification introduced to the cell by the base editors, compositions and methods of the present disclosure can be such that the cell and progeny of the cell are altered for improved production of biologic products such as an antibody, starch, alcohol or other desired cellular output. The modification introduced to the cell by the methods of the present disclosure can be such that the cell and progeny of the cell include an alteration that changes the biologic product produced.

The system can comprise one or more different vectors. In an aspect, the Cas9 is codon optimized for expression the desired cell type, preferentially a eukaryotic cell, preferably a mammalian cell or a human cell.

In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/(visited Jul. 9, 2002), and these tables can be adapted in a number of ways. See, Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen: Jacobus, Pa.), are also available. In some embodiments, one or more codons (e.g. 1, 2, 3, 4, 5, 10, 15, 20, 25, 50, or more, or all codons) in a sequence encoding an engineered nuclease correspond to the most frequently used codon for a particular amino acid.

Packaging cells are typically used to form virus particles that are capable of infecting a host cell. Such cells include 293 cells, which package adenovirus, and psi.2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA can be packaged in a cell line, which contains a helper plasmid encoding the other AAV genes, namely rep and cap, but lacking ITR sequences. The cell line can also be infected with adenovirus as a helper. The helper virus can promote replication of the AAV vector and expression of AAV genes from the helper plasmid. The helper plasmid in some cases is not packaged in significant amounts due to a lack of ITR sequences. Contamination with adenovirus can be reduced by, e.g., heat treatment to which adenovirus is more sensitive than AAV.

### Pharmaceutical Compositions

Other aspects of the present disclosure relate to pharmaceutical compositions comprising CRISPR system (e.g., including Cas9 disclosed herein). The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g., for specific delivery, increasing half-life, or other therapeutic compounds).

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.).

Some nonlimiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient,” “carrier,” “pharmaceutically acceptable carrier,” “vehicle,” or the like are used interchangeably herein.

Pharmaceutical compositions can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions can also contain one or more osmotic modulating agents, i.e., a compound that modulates the osmotic properties (e.g, tonicity, osmolality, and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

In some embodiments, the pharmaceutical composition is formulated for delivery to a subject, e.g., for gene editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseous, periocular, intratumoral, intracerebral, and intracerebroventricular administration.

In some embodiments, the pharmaceutical composition described herein is administered locally to a diseased site. In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump can be used (See. e.g., Langer, 1990, Science 249; 1527-1533; Sefton, 1989, CRC Crit. Ref. Biomed. Eng. 14:201; Buchwald et al. 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228: 190; During et al., 1989, Ann. Neurol. 25:351; Howard et ah, 1989, J. Neurosurg. 71: 105.) Other controlled release systems are discussed, for example, in Langer, supra.

In some embodiments, the pharmaceutical composition is formulated in accordance with routine procedures as a composition adapted for intravenous or subcutaneous administration to a subject. e.g., a human. In some embodiments, pharmaceutical composition for administration by injection are solutions in sterile isotonic use as solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration can be a liquid, e.g., sterile saline, lactated Ringer's or Hank's solution. In addition, the pharmaceutical composition can be in solid forms and re-dissolved or suspended immediately prior to use. Lyophilized forms are also contemplated. The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid diolcoylphosphatidylethanolamine (DOPE), low levels (5-10 mol %) of cationic lipid, and stabilized by a polyethyleneglycol (PEG) coating (Zhang Y. P. et ah, Gene Ther. 1999, 6: 143847). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See. e.g., U.S. Pat. Nos. 4,880,635; 4,906,477; 4,911,928; 4,917,951; 4,920,016; and 4,921,757; each of which is incorporated herein by reference.

The pharmaceutical composition described herein can be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile used for reconstitution or dilution of the lyophilized compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers can be formed from a variety of materials such as glass or plastic. In some embodiments, the container holds a composition that is effective for treating a disease described herein and can have a sterile access port. For example, the container can be an intravenous solution bag or a vial having a stopper pierceable by a hypodermic injection needle. The active agent in the composition is a compound of the invention. In some embodiments, the label on or associated with the container indicates that the composition is used for treating the disease of choice. The article of manufacture can further comprise a second container comprising a pharmaceutically-acceptable buffer, such as phosphate-buffered saline, Ringer's solution, or dextrose solution. It can further include other materials desirable from a commercial and user standpoint, including other buffers, diluents, filters, needles, syringes, and package inserts with instructions for use.

In some embodiments, the CRISPR system (e.g., including the Cas9 described herein) are provided as part of a pharmaceutical composition. In some embodiments, the pharmaceutical composition comprises any of the fusion proteins provided herein (e.g., including the nucleobase editor described herein comprising LubCas9). In some embodiments, the pharmaceutical composition comprises any of the complexes provided herein. In some embodiments, the pharmaceutical composition comprises a ribonucleoprotein complex comprising an RNA-guided nuclease (e.g., Cas9) that forms a complex with a gRNA and a cationic lipid. In some embodiments pharmaceutical composition comprises a gRNA, a nucleic acid programmable DNA binding protein, a cationic lipid, and a pharmaceutically acceptable excipient. Pharmaceutical compositions can optionally comprise one or more additional therapeutically active substances.

### Kits

In one aspect, the invention provides kits containing any one or more of the elements disclosed in the above methods and compositions. In some embodiments, the kit comprises a vector system and instructions for using the kit. In some embodiments, the vector system comprises one or more insertion sites for inserting a guide sequence, wherein when expressed, the guide sequence directs sequence-specific binding of a CRISPR complex to a target sequence in a eukaryotic cell, wherein the CRISPR complex comprises a CRISPR enzyme complexed with (1) the guide sequence that is hybridized to the target sequence, and (2) a sequence that is hybridized to the tracr sequence; and/or (b) a second regulatory element operably linked to an enzyme-coding sequence encoding said CRISPR enzyme comprising a nuclear localization sequence. Elements may be provide individually or in combinations, and may be provided in any suitable container, such as a vial, a bottle, or a tube. In some embodiments, the kit includes instructions in one or more languages, for example in more than one language.

In some embodiments, the kit comprises a nucleobase editor. For example, in some embodiments, the kit includes a nucleobase editor comprising the Lachnospira UBA3212 Cas9 (LubCas9) described herein.

In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more oligonucleotides corresponding to a guide sequence for insertion into a vector so as to operably link the guide sequence and a regulatory element. In some embodiments, the kit comprises a homologous recombination template polynucleotide.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

### EXAMPLES

The following examples describe some of the preferred modes of making and practicing the present invention. However, it should be understood that these examples are for illustrative purposes only and are not meant to limit the scope of the invention.

### Example 1. Screening for Novel Cas9 Enzymes, Discovery and Optimization of a Novel Cas9 from Lachnospira Bacterium

This example describes a screen for the discovery of novel Cas9 enzymes. As described herein, using this screen a novel Cas9 from Lachnospira bacterium was isolated and optimized.

In a search to discover new Cas9 enzymes which recognize novel PAM sequences, a bioinformatics screen was used to search for additional enzymes to expand CRISPR's targeting range. The screen utilized seed sequences of Cas9 from the S. pyogenes, S. aureus, S. thermophilus, and F. novicida. Bioinformatics was carried out using the tblastn variant of BLAST with an e-value threshold of 1e-6 for considering BLAST hits. Briefly, loci selected for testing were loci that remained intact in the presence of Cas9 proteins from other species. Loci were selected that had greater than three spacers within the CRISPR array and greater than 1 kb endogenous sequence 5′ of Cas9 and greater than 300 nt 3′ of the CRISPR array. Using this approach, a novel Cas9 enzyme was identified from Lachnospira species and codon optimized for expression in human cells. This novel engineered Cas9 was then recombinantly produced and tested.

### Example 2. Identifying 3° PAM Consensus Motif for Lachnospira UBA3212 Cas9

This example illustrates the identification of the protospacer adjacent motif (PAM) sequence for human codon-optimized Lachnospira UBA3212 Cas9 originally isolated from Lachnospira species.

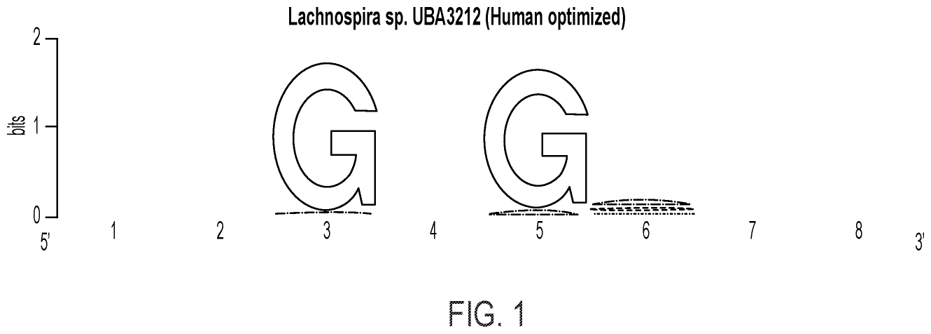

The human, codon-optimized Cas9 was tested for its recognition of a PAM sequence using an in vitro PAM identification assay. A library of plasmids bearing randomized PAM sequences were incubated with Lachnospira UBA3212 Cas9. Uncleaved plasmid was purified and sequenced to identify specific PAM motifs that were cleaved. The consensus PAM sequence recognized by Lachnospira UBA3212 Cas9 was identified as 5′-NNGNG-3′ (FIG. 1).

### Example 3. RNA Folding Structure of crRNA, tracrRNA and sgRNA for Lachnospira UBA3212 Cas9

This example demonstrates the predicted RNA folding structure of exemplary crRNA, tracrRNA, and sgRNA for use with Lachnospira UBA3212 Cas9. This example also shows various tested sgRNAs (sgRNAs 1-11) used with Lachnospira UBA3212 Cas9.

Small RNA sequencing was carried out on RNA derived from an E. coli strain heterologously expressing Lachnospira UBA3212 Cas9 Crispr loci. Briefly, RNA was isolated from stationary phase bacteria by first resuspending the E. coli in Trizol, then homogenizing the bacteria with zirconia/silica beads in a homogenizer for three 1 min cycles. Total RNA was purified from homogenized samples, DNAse treated and 3′ dephosphorylated with T4 polynucleotide kinase and rRNA was removed. RNA libraries were prepared from rRNA-depleted RNA, and size selected for small RNA.

For RNA sequencing, transcripts were poly-A tailed with E. coli Poly (A) polymerase, ligated with 5′ RNA adapters using T4 RNA ligase 1 and reverse transcribed, followed by PCR amplification of cDNA with barcoded primers, and sequencing on a MiSeq. Reads from each sample were identified on the basis of their associated barcode and aligned to a reference sequence using BWA. Paired-end alignments were used to extract transcript sequences using Picard tools and the sequences were analyzed using Geneious software.

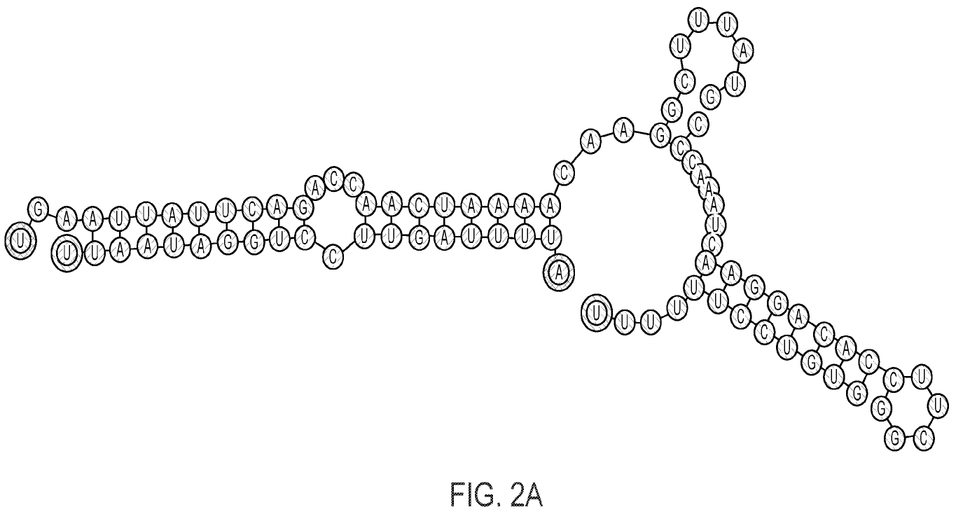

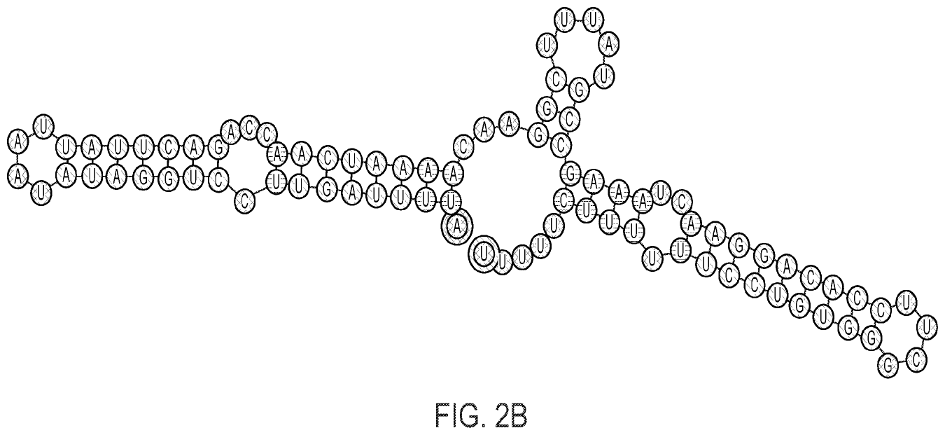

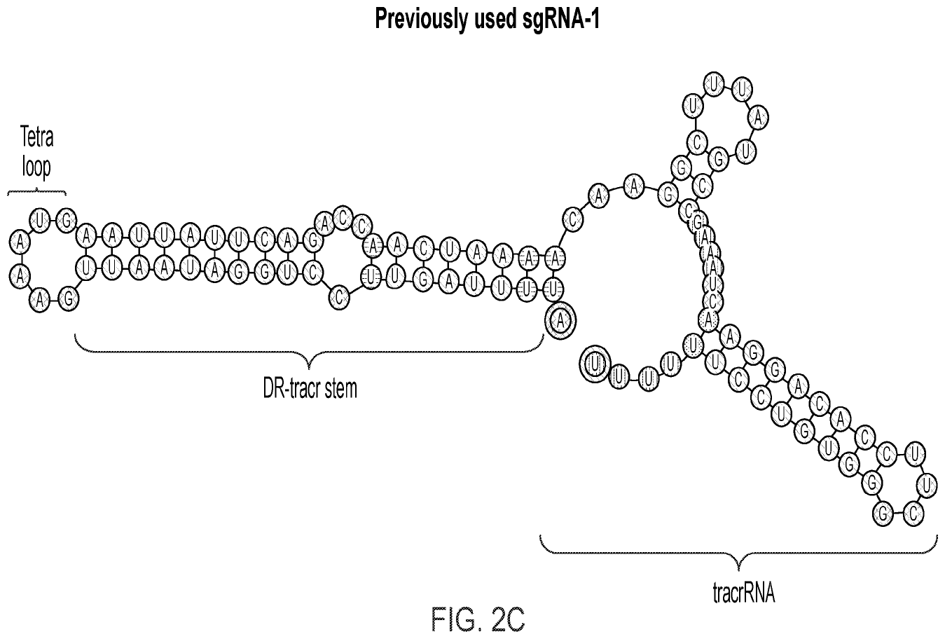

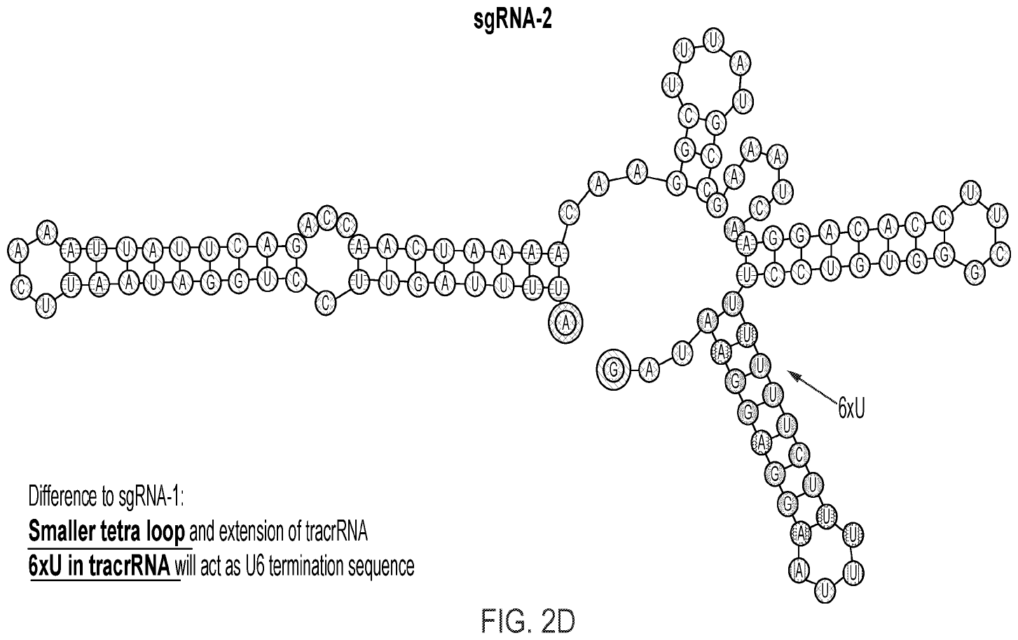

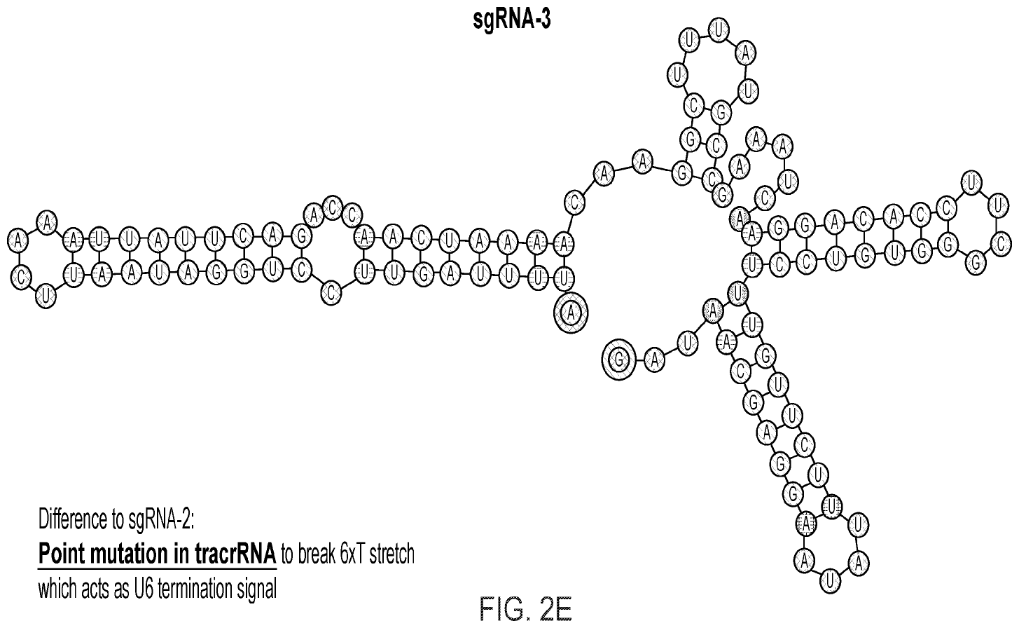

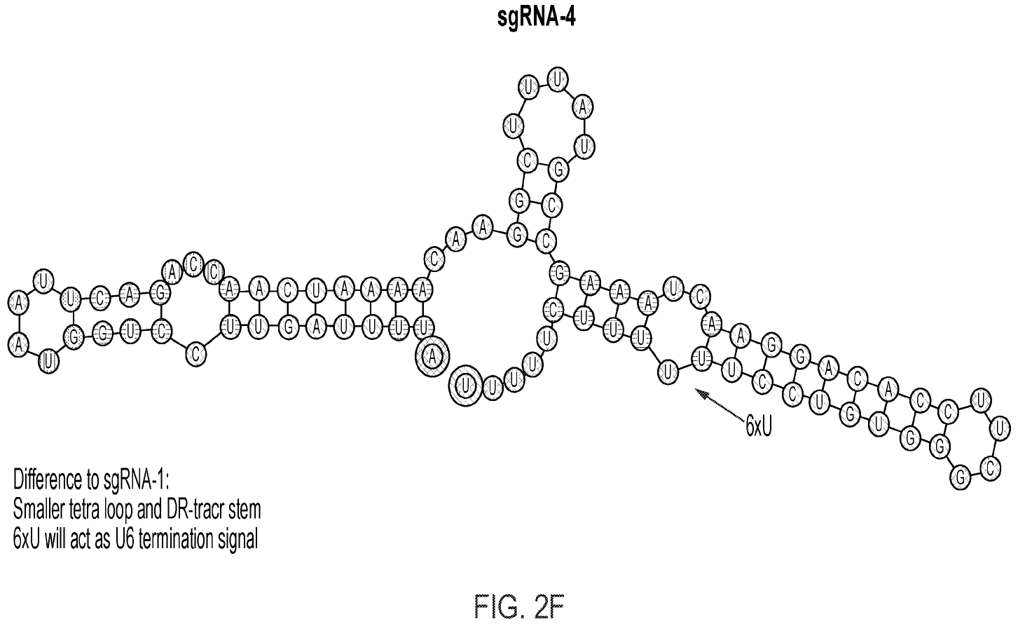

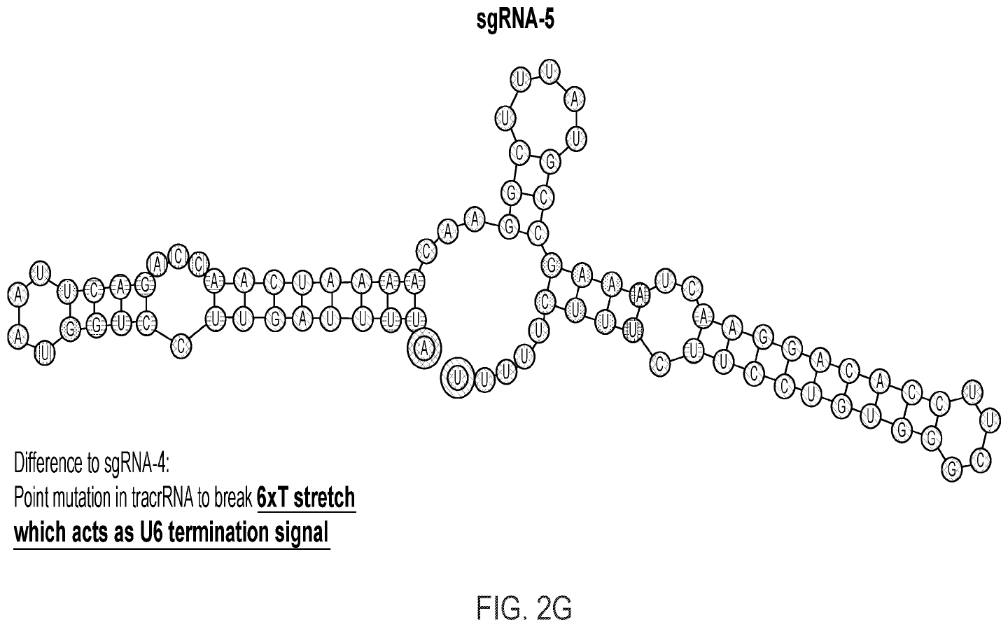

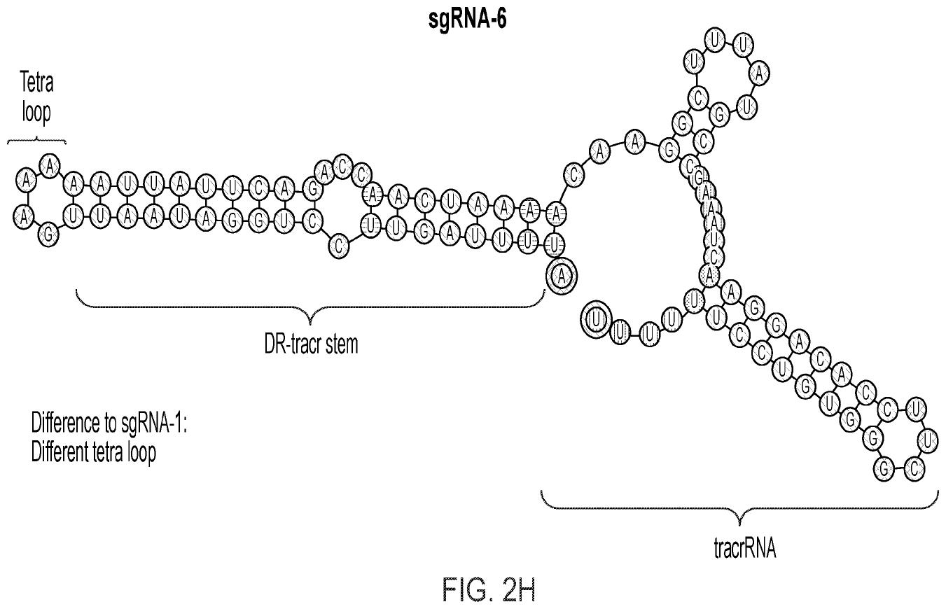

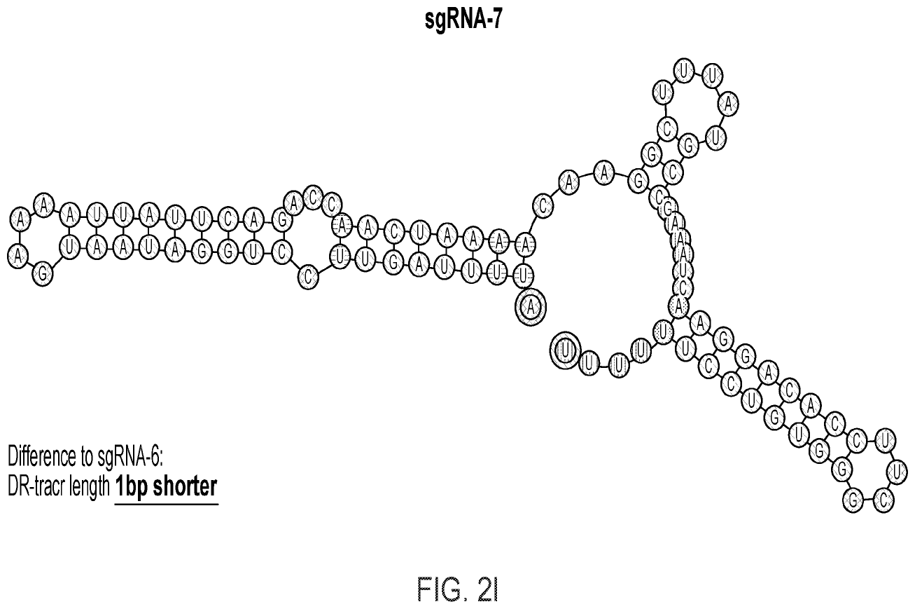

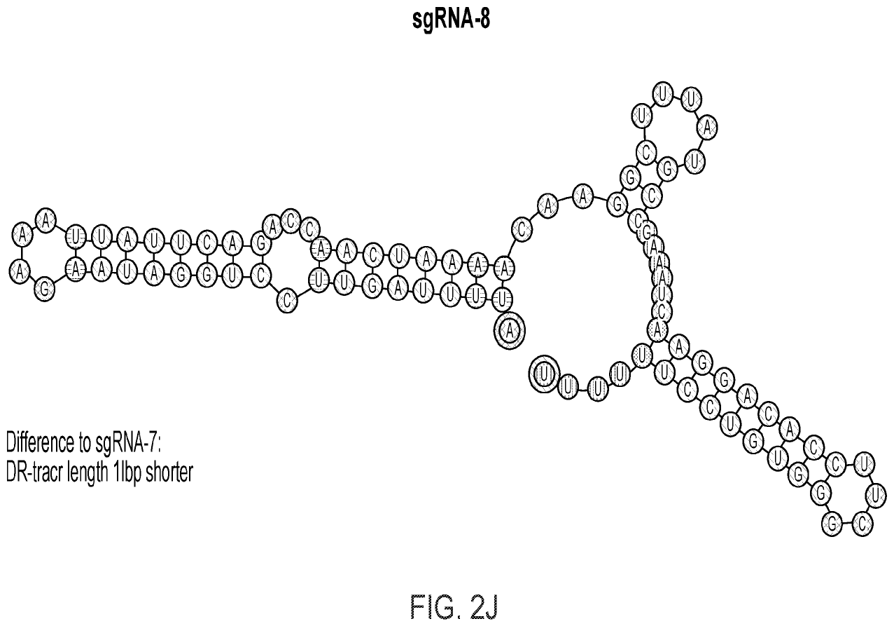

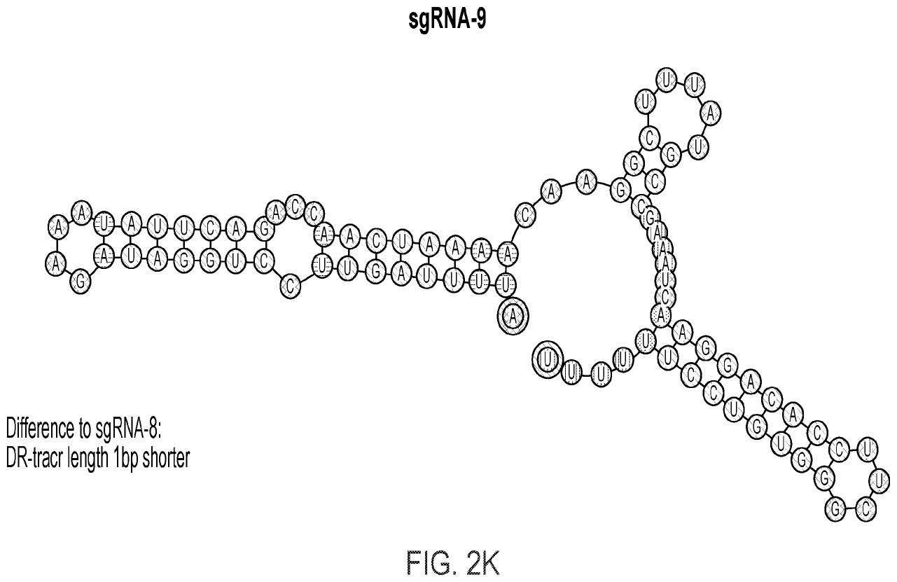

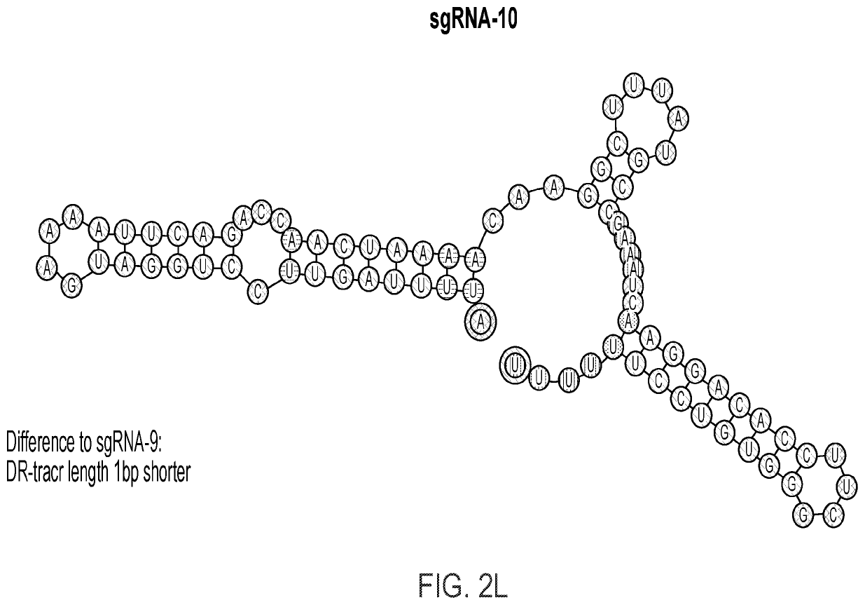

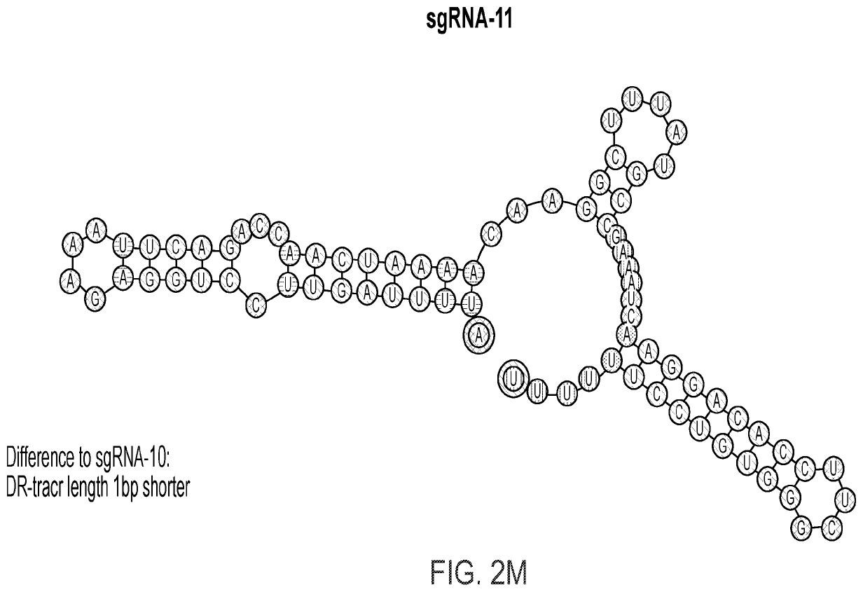

RNA folding was based on prediction from Geneious 11.1.2 software. The predicted RNA folding structure for crRNA and tracrRNA is shown in FIG. 2A. The predicted RNA folding structure for the chimeric sgRNA is shown in FIG. 2B. The single sgRNA transcript fuses the crRNA to tracrRNA mimicking the dual RNA structure required to guide site-specific UBA3212 Cas9 activity.

A set of 11 sgRNA sequences were created and tested for use with Lachnospira UBA3212 Cas9 (FIG. 2C). The sequences for each of these sgRNAs is provided in Table 8 below. For these studies, RNA from E. coli heterologously expressing a minimal LubCas9 CRISPR locus was used for small RNA sequencing (RNAseq). CrRNA and tracrRNA were determined from small RNAseq reads. RNA folding of crRNA with tracr RNA was predicted through the use of Geneious software (geneious.com).

Table 8 shows exemplary Lachnospira UBA3212 Cas9crRNA, tracrRNA and sgRNA sequences.

TABLE 8
Exemplary Lachnospira UBA3212 Cas9crRNA, tracrRNA
and sgRNA sequences

| Sequence ID No. | crRNA |
|---|---|
| (description) | |
| SEQ ID NO:3 (Full- | AUUUUAGUUCCUGGAUAAUUCAAGUUAGUGUAAAAC |
| length Direct Repeat | |
| CIRNA Sequence) | |
| SEQ ID NO: 4 (22 nt | AUUUUAGUUCCUGGAUAAUUCA |
| Direct Repeat crRNA | |
| Sequence) | |
| SEQ ID NO: 5 (Mature | NNNNNNNNNNNNNNNNNNNNAUUUUAGUUCCUGGAUAAUUCA |
| crRNA Sequence) | |
| SEQ ID NO: 6 | UGAAUUAUUCAGACCAACUAAAACAAGGCUUUAUGCCGAAAU |
| (Predicted tracrRNA | CAAGGACACCUUCGGGUGUCCUUUUUU |
| Sequence) | |
| SEQ ID NO: 7 | AUUUUAGUUCCUGGAUAUAAUUAUUCAGACCAACUAAAACAA |
| (Predicted sgRNA | GGCUUUAUGCCGAAAUCAAGGACACCUUCGGGUGUCCUUUUU |
| scaffold) | UCUUUUU |
| | Direct repeat 22 nt crRNA (bold) |
| | Tetra loop (underlined) |
| | TracrRNA |
| SEQ ID NO: 13 | AUUUUAGUUCCUGGAUAAUUGAAAUGAAUUAUUCAGACCAAC |
| (sgRNA-1) | UAAAACAAGGCUUUAUGCCGAAAUCAAGGACACCUUCGGGUG |
| | UCCUUUUUU |
| SEQ ID NO: 14 | AUUUUAGUUCCUGGAUAAUUCAAAUUAUUCAGACCAACUAAA |
| (sgRNA-2) | ACAAGGCUUUAUGCCGAAAUCAAGGACACCUUCGGGUGUCCU |
| | UUUUUCUUUUUAAGGAGGAAUAG |
| SEQ ID NO: 15 | AUUUUAGUUCCUGGAUAAUUCAAAUUAUUCAGACCAACUAAA |
| (sgRNA-3) | ACAAGGCUUUAUGCCGAAAUCAAGGACACCUUCGGGUGUCCU |
| | UUGUUCUUUAUAAGGAGCAAUAG |
| SEQ ID NO: 16 | AUUUUAGUUCCUGGUAAUUCAGACCAACUAAAACAAGGCUUU |
| (sgRNA-4) | AUGCCGAAAUCAAGGACACCUUCGGGUGUCCUUUUUUCUUUU |
| | U |
| SEQ ID NO: 17 | AUUUUAGUUCCUGGUAAUUCAGACCAACUAAAACAAGGCUUU |
| (sgRNA-5) | AUGCCGAAAUCAAGGACACCUUCGGGUGUCCUUCUUUCUUUU |
| | U |
| SEQ ID NO: 18 | AUUUUAGUUCCUGGAUAAUUGAAAAAUUAUUCAGACCAACUA |
| (sgRNA-6) | AAACAAGGCUUUAUGCCGAAAUCAAGGACACCUUCGGGUGUC |
| | CUUUUUU |
| SEQ ID NO: 19 | AUUUUAGUUCCUGGAUAAUGAAAAUUAUUCAGACCAACUAAA |
| (sgRNA-7) | ACAAGGCUUUAUGCCGAAAUCAAGGACACCUUCGGGUGUCCU |
| | UUUUU |
| SEQ ID NO: 20 | AUUUUAGUUCCUGGAUAAGAAAUUAUUCAGACCAACUAAAAC |
| (sgRNA-8) | AAGGCUUUAUGCCGAAAUCAAGGACACCUUCGGGUGUCCUUU |
| | UUU |
| SEQ ID NO: 21 | AUUUUAGUUCCUGGAUAGAAAUAUUCAGACCAACUAAAACAA |
| (sgRNA-9) | GGCUUUAUGCCGAAAUCAAGGACACCUUCGGGUGUCCUUUUU |
| SEQ ID NO: 22 | AUUUUAGUUCCUGGAUGAAAAUUCAGACCAACUAAAACAAGG |
| (sgRNA-10) | CUUUAUGCCGAAAUCAAGGACACCUUCGGGUGUCCUUUUUU |
| SEQ ID NO: 23 | AUUUUAGUUCCUGGAGAAAUUCAGACCAACUAAAACAAGGCU |
| (sgRNA-11) | UUAUGCCGAAAUCAAGGACACCUUCGGGUGUCCUUUUUU |

### Example 4. Measuring In Vitro Nucleic Acid Cleavage Activity by UBA3212 Cas9

This example shows demonstrable cleavage activity of target nucleic acids by Lachnospira UBA3212 Cas9.

HEK293T cells were transfected with human codon-optimized UBA3212 Cas9 or GFP (control). Whole cell lysates were prepared with lysis buffer (20 mM HEPES, 100 mM KCl, 5 mM MgCl2, 1 mM DTT, 5% glycerol, 0.1% Triton X-100) supplemented with protease inhibitors (Ran et al., 2015).

DNA substrates were generated by PCR amplification of pUC19 plasmids containing DNA fragments with the FnPSP1 sequence flanked by different 3′ PAM sequences.

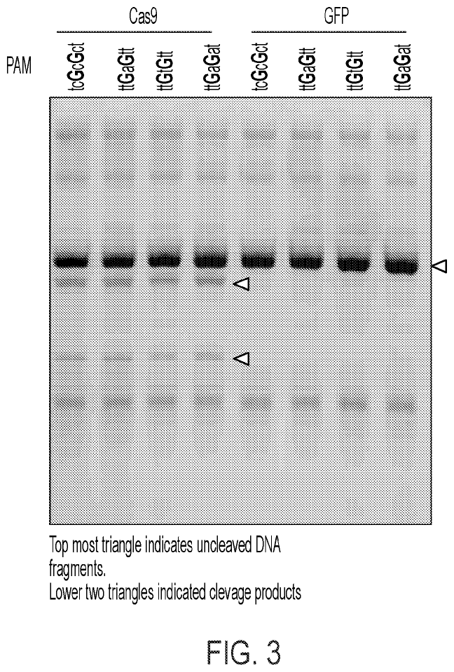

The in vitro cleavage assay was carried out by incubating the Cas9 containing whole cell lysate in cleavage buffer (100 mM HEPES, 500 mM KCl, 25 mM MgCl2, 5 mM DTT, 25% glycerol), supplemented with in vitro transcribed sgRNA targeting Fn protospacer 1 (FnPSP1) and in vitro generated DNA substrates containing the target FnPSP1 site. As a control, whole cell lysates obtained from cells transfected with GFP instead of Cas9 were used. After 30 min incubation, cleavage reactions were purified and treated with RNAse A at a final concentration of 80 ng/ul and analyzed on a 1% agarose gel (FIG. 3).

As seen in FIG. 3, human-codon optimized Cas9 shows demonstrable cleavage activity. Table 9 below shows the sequences that were used for the in vitro assays described in this example.

TABLE 9
Sequences for in vitro DNA cleavage assay

| Sequence ID No. | |
|---|---|
| (description) | Components of DNA cleavage assay |
| SEQ ID NO: 8 (Fn | CAUUUAAUAAGGCCACUGUUAAA |
| protospacer 1 guide | |
| Sequence) | |
| SEQ ID NO: 9 (sgRNA | CAUUUAAUAAGGCCACUGUUAAAAUUUUAGUUCCUGGAUAUA |
| Sequence) | AUUAUUCAGACCAACUAAAACAAGGCUUUAUGCCGAAAUCAA |
| | GGACACCUUCGGGUGUCCUUUUUUCUUUUU |
| SEQ ID NO: 10 (PCR | ACACATGCAGCTCCCGGAGACGGTCACAGCTTGTCTGTAAGCGG |
| amplified DNA | ATGCCGGGAGCAGACAAGCCCGTCAGGGCGCGTCAGCGGGTGT |
| targets) | TGGCGGGTGTCGGGGCTGGCTTAACTATGCGGCATCAGAGCAGA |
| | TTGTACTGAGAGTGCACCATATGCGGTGTGAAATACCGCACAGA |
| | TGCGTAAGGAGAAAATACCGCATCAGGCGCCATTCGCCATTCAG |
| | GCTGCGCAACTGTTGGGAAGGGCGATCGGTGCGGGCCTCTTCGC |
| | TATTACGCCAGCTGGCGAAAGGGGGATGTGCTGCAAGGCGATT |
| | AAGTTGGGTAACGCCAGGGTTITCCCAGTCACGACGTTGTAAAA |
| | CGACGGCCAGTGAATTCGAGCTCGGTACCCGGGGATCCGAGAA |
| | GTCATTTAATAAGGCCACTGTTAAANNNNNNNAAGCTTGGCGT |
| | AATCATGGTCATAGCTGTTTCCTGTGTGAAATTGTTATCCGCTCA |
| | CAATTCCACACAACATACGAGCCGGAAGCATAAAGTGTAAAGC |
| | CTGGGGTGCCTAATGAGTGAGCTAACTCACATTAATTGCGTTGC |
| | GCTCACTGCCCGCTTTCCAGTCG |
| | Fn protospacer 1 (FnPSP1) Sequence (Bold) |
| | PAM Sequences (Underlined) |

### Example 5. Ex Vivo Cleavage Activity by UBA3212 Cas9 in HEK293T Cells

This example illustrates ex vivo nucleic acid cleavage activity Lachnospira UBA3212 Cas9 in HEK293T cells.

HEK293T cells were plated in a 96-well plate. Cells were transfected with expression vectors containing Cas9 and guide RNAs (Table 10), 24 hours after plating. Cells were harvested 72 hours post-transfection and total DNA was extracted.

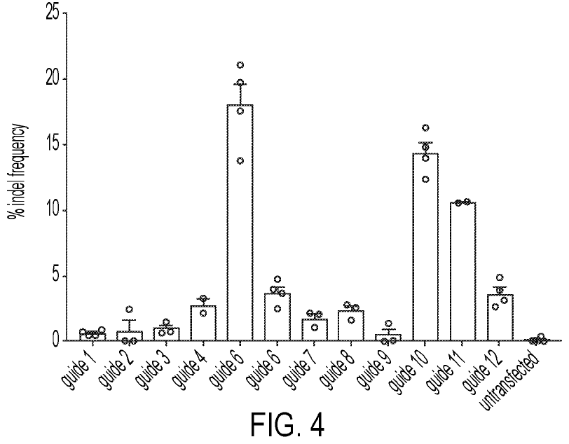

Deep sequencing was carried out to characterize indel patterns in the HEK293T cells. Briefly, exemplary targets (Table 10) were amplified using a two-round PCR region to add Illumina adapters as well as unique barcodes to the target amplicons. PCR products were run on a 2% gel and gel extracted. Samples were pooled, quantified and cDNA libraries were prepared and sequenced on MiSeq. Indel frequency was determined by deep sequencing (FIG. 4).

TABLE 10
Guide RNA Sequences and PAM Sequences

| ID | 5′->3′ sequence | 3′PAM |
|---|---|---|
| guide 1 | TAGAACCCTCTGGGGACCGTTTG | AGGAG |
| | (SEQ ID NO: 88) | |
| guide 2 | CCTGTCAAGTGGCGTGACACCGG | GCGTG |
| | (SEQ ID NO: 89) | |
| guide 3 | TTTCCCTTCAGCTAAAATAAAGG | AGGAG |
| | (SEQ ID NO: 90) | |
| guide 4 | CATTATATCAAATCTACCACTGT | ATGAG |
| | (SEQ ID NO: 91) | |
| guide 5 | CTGTGCCCCTCCCTCCCTGGCCC | AGGTG |
| | (SEQ ID NO: 92) | |
| guide 6 | GACAAAGTACAAACGGCAGAAGC | TGGAG |
| | (SEQ ID NO: 93) | |
| guide 7 | AGGGCTCCCATCACATCAACCGG | TGGCG |
| | (SEQ ID NO: 94) | |
| guide 8 | GGGCAACCACAAACCCACGAGGG | CAGAG |
| | (SEQ ID NO: 95) | |
| guide 9 | TGCAGAGCAAATACCAGAGATAA | GAGAG |
| | (SEQ ID NO: 96) | |
| guide 10 | GGGAGGTCAGAAATAGGGGGTCC | AGGAG |
| | (SEQ ID NO: 97) | |
| guide 11 | GTGTGCAGACGGCAGTCACTAGG | GGGCG |
| | (SEQ ID NO: 98) | |
| guide 12 | CCCCCTTCAATATTCCTAGCAAA | GAGGG |
| | (SEQ ID NO: 99) | |

### Example 6. Base Editing by Lachnospira UBA3212 Cas9 (D8A Mutant) Enzyme with an N-Terminal Fusion of TadA8 Adenosine Deaminase

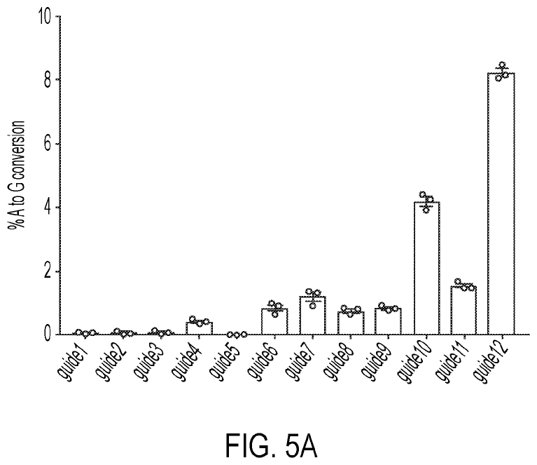

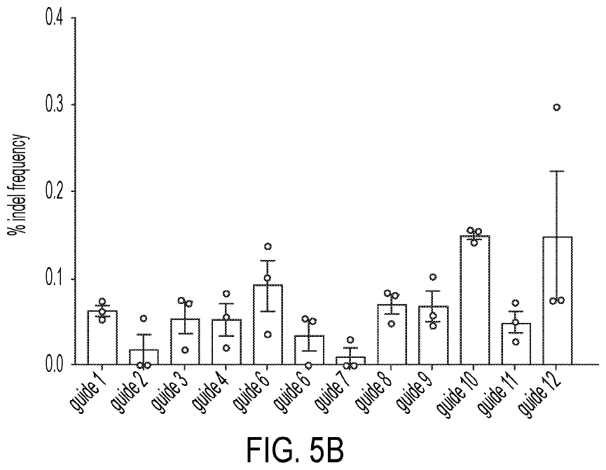

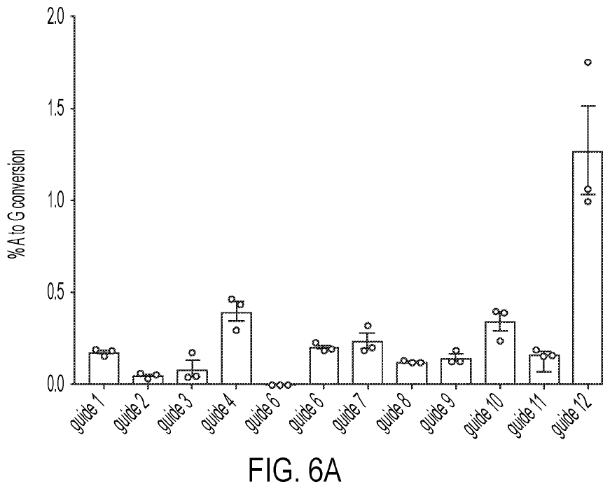

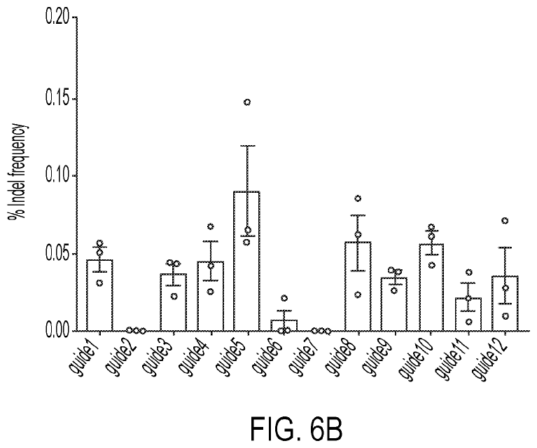

This example illustrates base conversion efficiency of a Lachnospira UBA3212 Cas9 D8A mutant enzyme (“LubCas9 (D8A)”) fused to an adenine base editor, TadA8. FIGS. 5A and 6A show graphs of targeted adenine to guanine conversion percentage achieved with an N-terminal fusion (FIG. 5A, SEQ ID NO: 11) and a C-terminal fusion (FIG. 6A, SEQ ID NO: 12) of TadA8, an adenosine deaminase, with LubCas9 (D8A), using the guide RNAs at Table 12, which are directed to genomic sites in a human cell line (HEK293T).

TABLE 11
Sequences for exemplary Cas9 adenosine base editors

| | |
|---|---|
| Sequence ID No. | |
| (description) | Components of DNA cleavage assay |

Sequence of Adenine Deaminase, TadA8 fused to the N-terminal of
Lachnospira UBA3212 Cas9 (D8A mutant)
MPAAKRVKLDGSEVEFSHEYWMRHALTLAKRARDEREVPVGAVLVLNNRVIGEGWNRAIGLHDP
TAHAEIMALROGGLVMQNYRLYDATLYVTFEPCVMCAGAMIHSRIGRVVFGVRNAKTGAAGSLM
DVLHHPGMNHRVEITEGILADECAALLCRFFRMPRRVFNAQKKAQSSTDGSSGSETPGTSESAT
PESSGPKKKRKVGSVNVGL [symbol] IGIASVGVAVVDSESGEILEAVSDLFESAEANQNVDRRGFRQSR
RLKRRQYNRIHDFMKLWEEFGFVKPENINLNTVGLRVKSLTEQVTLDELYVILLSELKHRGISY
LEDSEEVDGGSEYKEGLRINQRELQSKYPCEIQLERLKIYGRYRGNFTVEIDGEKVGLSNVFTT
GAYRKEIQQLLSIQKTYQSKLTDDFINKYLEIFDRKRQYYVGPGNEKSRTDYGRYTTKKDAEGN
YITDENIFEKLIGKCSIYPEEMRAAGASYTAQEFNLLNDLNNLTIGGRKIEEEEKRAIIETIKS
SKVVNVEKIICKVTGEDAETITGARIDKDDKRIYHSFECYRKLKKALETIEVKIEEYSREELDE
LARILTLNTEREGILGELEKSFLDLGEEVIDCVIDFRRKNGPLFSKWQSFSLRLMNDIIPDMYE
QPKEQMTLLTEMGLMKSKKEIFKGMKYIPENVMRDDIYNPVVVRSVRIAVRALNAVIKKYGEID
KVVIEMPRDRNTEEQKKRIDAENKRNREELPGIEKRILEEYGIKITSAHYRNHKQLGLKLKLWN
EQGGICPYSGKTIDLERLLQNAGDYEVDHIIPLSISLDDSRNNKVLVYASENQKKGNQTPYAYL
SSVQREWGWEQYRHYVLSDLKKKKISSKKIENYLFMKDISKIDVVKGFIQRNLNDTRYASKVVL
NTLESFFKANEKETKVSVIRGSFTSLMRKNLKLDKSREESYAHHAVDALLIAYSKMGYDSYHKL
QGEFIDFETGEILDSRMWETNLEPDILKGYLYGRKWSEIRENIKIAESRVKYWHMTNKKCNRSL
CNQTLYGTRTYDGKIYQIKKIKDIRTPEGLKTFKDLVDKNKGDHLLMARNDPKTYEQILQIYRD
YSDAKNPFLQYEMETGDCIRKYSKKHNGSRIVSLKYHDGEVNSCIDVSHKYGFEKGSQKVVLMS
LNPYRMDVYKNCNDGKYYLIGLKQSDIKCEGRHYVIDEEKYAKVLVNEKMIQPGQSRKDLPDLG
YEFVMSFYKNEIIQYEKDGKFYKERFLSRTKPASRNYIETKPVDKPNFEKRHQIGLAKTTFIRK
IRTDILGNEYNCDREKFSSICKRPAATKKAGQAKKKKGSYPYDVPDYAYPYDVPDYAYPYDVPD
YA (SEQ ID NO: 11)
Sequence of Adenine Deaminase, TadA8 fused to the C-terminal of
Lachnospira UBA3212 Cas9 (D8A mutant)
MPKKKRKVGSVNVGL [symbol] IGIASVGVAVVDSESGEILEAVSDLFESAEANQNVDRRGFRQSRRLKR
RQYNRIHDFMKLWEEFGFVKPENINLNTVGLRVKSLTEQVTLDELYVILLSELKHRGISYLEDS
EEVDGGSEYKEGLRINQRELQSKYPCEIQLERLKIYGRYRGNFTVEIDGEKVGLSNVETTGAYR
KEIQQLLSIQKTYQSKLTDDFINKYLEIFDRKRQYYVGPGNEKSRTDYGRYTTKKDAEGNYITD
ENIFEKLIGKCSIYPEEMRAAGASYTAQEFNLLNDLNNLTIGGRKIEEEEKRAIIETIKSSKVV
NVEKIICKVTGEDAETITGARIDKDDKRIYHSFECYRKLKKALETIEVKIEEYSREELDELARI
LTLNTEREGILGELEKSFLDLGEEVIDCVIDERRKNGPLFSKWQSFSLRLMNDIIPDMYEQPKE
QMTLLTEMGLMKSKKEIFKGMKYIPENVMRDDIYNPVVVRSVRIAVRALNAVIKKYGEIDKVVI
EMPRDENTEEQKKRIDAENKRNREELPGIEKRILEEYGIKITSAHYRNHKQLGLKLKLWNEQGG
ICPYSGKTIDLERLLONAGDYEVDHIIPLSISLDDSRNNKVLVYASENQKKGNQTPYAYLSSVQ
REWGWEQYRHYVLSDLKKKKISSKKIENYLFMKDISKIDVVKGFIQRNLNDTRYASKVVLNTLE
SFFKANEKETKVSVIRGSFTSLMRKNLKLDKSREESYAHHAVDALLIAYSKMGYDSYHKLQGEF
IDFETGEILDSRMWETNLEPDILKGYLYGRKWSEIRENIKIAESRVKYWHMTNKKCNRSLCNQT
LYGTRTYDGKIYQIKKIKDIRTPEGLKTEKDLVDKNKGDHLLMARNDPKTYEQILQIYRDYSDA
KNPFLQYEMETGDCIRKYSKKHNGSRIVSLKYHDGEVNSCIDVSHKYGFEKGSQKVVLMSLNPY
RMDVYKNCNDGKYYLIGLKQSDIKCEGRHYVIDEEKYAKVLVNEKMIQPGQSRKDLPDLGYEEV
MSFYKNEIIQYEKDGKFYKERFLSRTKPASRNYIETKPVDKPNFEKRHQIGLAKTTFIRKIRTD
ILGNEYNCDREKFSSICKRPAATKKAGQAKKKKSGSETPGTSESATPESSGSEVEFSHEYWMRH
ALTLAKRARDEREVPVGAVLVLNNRVIGEGWNRAIGLHDPTAHAEIMALROGGLVMQNYRLYDA
TLYVTFEPCVMCAGAMIHSRIGRVVFGVRNAKTGAAGSLMDVLHHPGMNHRVEITEGILADECA
ALLCRFFRMPRRVFNAQKKAQSSTDPAAKRVKLDGSYPYDVPDYAYPYDVPDYAYPYDVPDYA
(SEQ ID NO: 12)
NLS (bold)
Linker (underlined, no italics or bolding)

TadA8 (italics and underlined)
D8A mutation in LubCas9 (bold and italics)
3×HA tag (italics), can be substituted with different tags

The TadA8 adenosine deaminase enzyme catalyzes the deamination of adenine to inosine, which is read as guanine by the translational machinery. Fusion of TadA8 to LubCas9 (D8A) directs base editing at loci recognized and targeted by the Cas9 gene editing system.

Briefly, 25,000 HEK293T cells were plated per 96-well. 100 ng of Cas9 expression plasmid and 100 ng of guide expression plasmid were transfected 24 h after plating. Cells were harvested 5 days after transfection and DNA was extracted.

Deep sequencing was carried out to characterize A-to-G conversion in the HEK293T cells. As described in Example 5, exemplary targets (Table 12) were amplified using a two-round PCR region to add Illumina adapters as well as unique barcodes to the target amplicons. PCR products were run on a 2% gel and gel extracted. Samples were pooled, quantified and cDNA libraries were prepared and sequenced on MiSeq. The percent A-to-G conversion was determined by deep sequencing for the N-terminal TadA8-LubCas9 (D8A) fusion (FIG. 5A) as well as the C-terminal LubCas9 (D8A)-TadA8 fusion constructs (FIG. 6A).

TABLE 12
Guide RNA Sequences Depicting Adenine
Residues (highlighted in bold)
Targeted for A to G Conversion
and PAM Sequences

| ID | 5′->3′ sequence | 3′PAM |
|---|---|---|
| guide 1 | TAGAACCCTCTGGGGACCGTTTG | AGGAG |
| | (SEQ ID NO: 88) | |
| guide 2 | CCTGTCAAGTGGCGTGACACCGG | GCGTG |
| | (SEQ ID NO: 89) | |
| guide 3 | TTTCCCTTCAGCTAAAATAAAGG | AGGAG |
| | (SEQ ID NO: 90) | |
| guide 4 | CATTATATCAAATCTACCACTGT | ATGAG |
| | (SEQ ID NO: 91) | |
| guide 5 | CTGTGCCCCTCCCTCCCTGGCCC | AGGTG |
| | (SEQ ID NO: 92) | |
| guide 6 | GACAAAGTACAAACGGCAGAAGC | TGGAG |
| | (SEQ ID NO: 93) | |
| guide 7 | AGGGCTCCCATCACATCAACCGG | TGGCG |
| | (SEQ ID NO: 94) | |
| guide 8 | GGGCAACCACAAACCCACGAGGG | CAGAG |
| | (SEQ ID NO: 95) | |
| guide 9 | TGCAGAGCAAATACCAGAGATAA | GAGAG |
| | (SEQ ID NO: 96) | |
| guide 10 | GGGAGGTCAGAAATAGGGGGTCC | AGGAG |
| | (SEQ ID NO: 97) | |
| guide 11 | GTGTGCAGACGGCAGTCACTAGG | GGGCG |
| | (SEQ ID NO: 98) | |
| guide 12 | CCCCCTTCAATATTCCTAGCAAA | GAGGG |
| | (SEQ ID NO: 99) | |

The data showed that both N-terminal and C-terminal fusion proteins of LubCas9 (D8A) with an adenine deaminase carried out base editing, and that the N-terminal fusion resulted in a higher frequency of A to G conversion, especially with guide RNAs 10, 11 and 12. Guide RNA 12 achieved A to G conversion of about 8%. Guide RNA 5 served as the negative control in the assay. Simultaneous with detection of A-to-G editing, indel frequency was also examined at each targeted site by cataloguing the sequence reads showing sequence insertions or deletions at the sites. Low levels of indels were observed with N-terminal (FIG. 5B, SEQ ID NO: 11) and C-terminal fusions (FIG. 6B, SEQ ID NO: 12) of TadA8 adenosine deaminase to LubCas9 (D8A). Desirably, base editors are capable of modifying a specific nucleotide base without generating a significant proportion of indels.

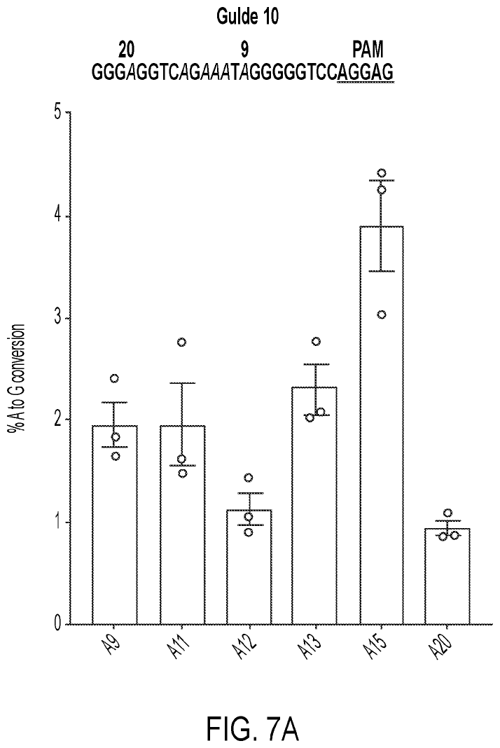

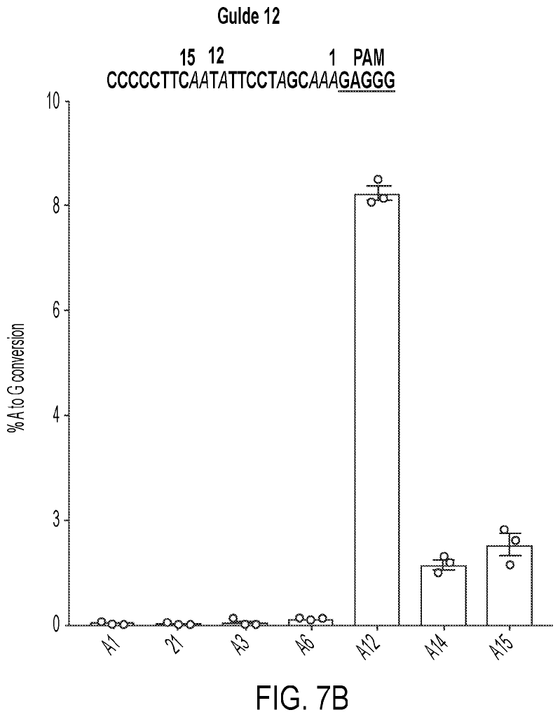

Base editors comprising adenosine deaminase fused to Cas9 (e.g., nickase or dead variants) convert A-to-G within a small editing window typically defined by the number of the nucleotides from the PAM sequence in which a particular base editor acts to induce efficient point mutations. The activity window for most base editors is typically <10 nucleotides wide. To examine the window for the base editor comprising TadA8 fused to the N-terminus of LubCas9, the A-to-G conversion rate of each adenosine residue was quantified from deep sequencing data. FIGS. 7A and 7B show graphs of the A to G conversion percentage achieved at each adenine residue using N-terminal fusion proteins of TadA8 to LubCas9 (D8A) (SEQ ID NO: 11) using guide RNA 10 (FIG. 7A) and guide RNA 12 (FIG. 7B).

For guide RNA 10, the base conversion percentage was greatest at residue A15 (˜4% A-to-G conversion). The other adenine residues within guide RNA 10 showed between about 1-2 percent conversion at this target site (FIG. 7A. Without being bound by theory, this provides a potentially broad activity window centered at or near A15. For guide RNA 12, the base conversion percentage was greatest at residue, A12 (˜8% A to G conversion). Additionally, A-to-G conversion between about 1-2 percent was obtained at residues A14 and A15. Residues A1, A2, A3 and A6 did not show any appreciable base editing, indicating that residues at these positions are unlikely to be in the window accessible by this base editor (FIG. 7B). Without being bound by theory, this suggests a range for which base editing may be optimal for this base editor.

### Example 7: LubCas9 Nuclease Activity with sgRNA of Different Guide Lengths

This example illustrates LubCas9 nuclease activity using the sgRNAs shown in Table 8. For these studies, LubCas9 nuclease activity was tested using sgRNAs having different designs and guide lengths.

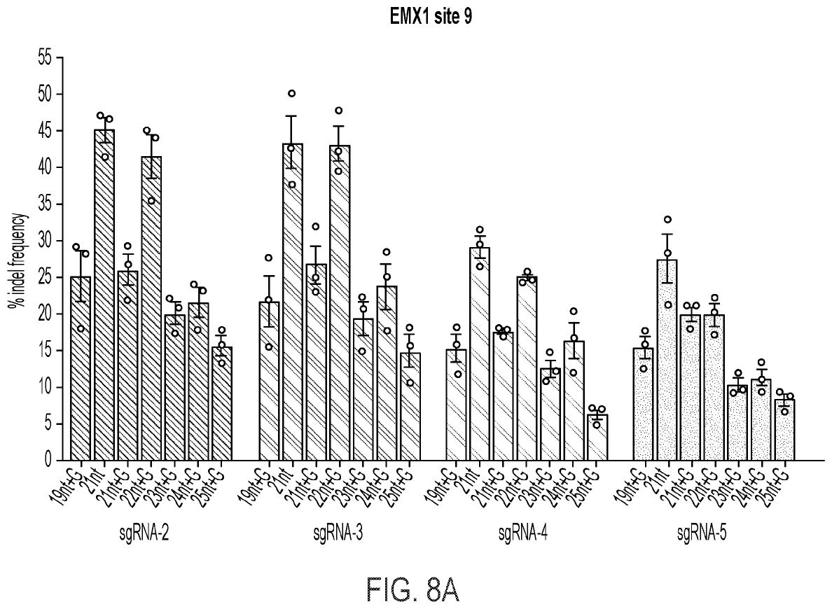

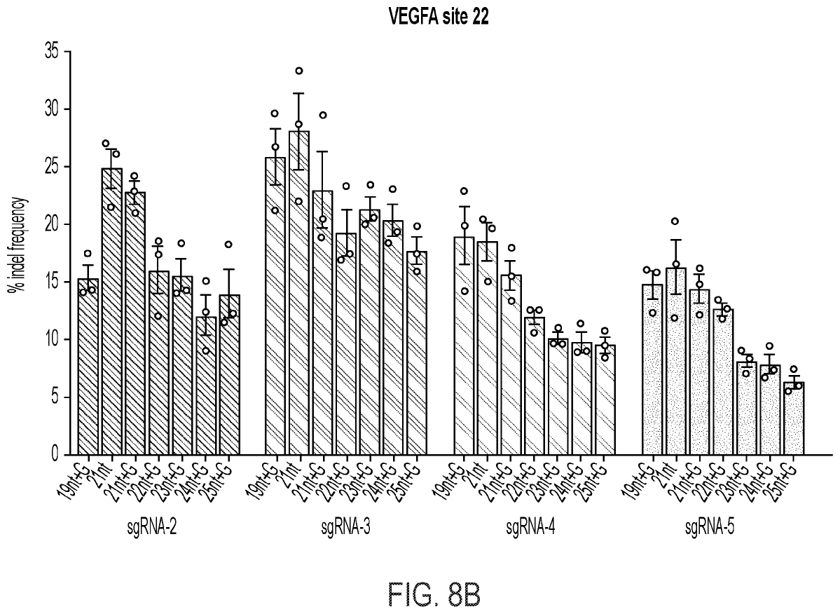

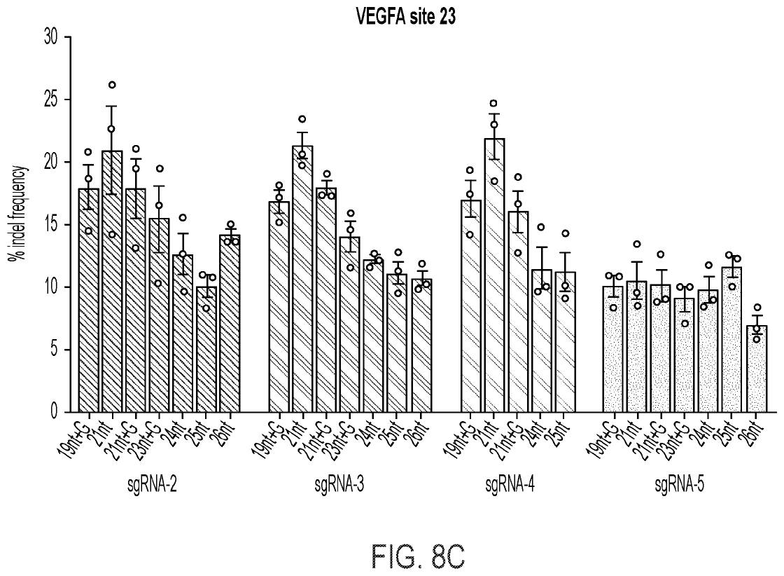

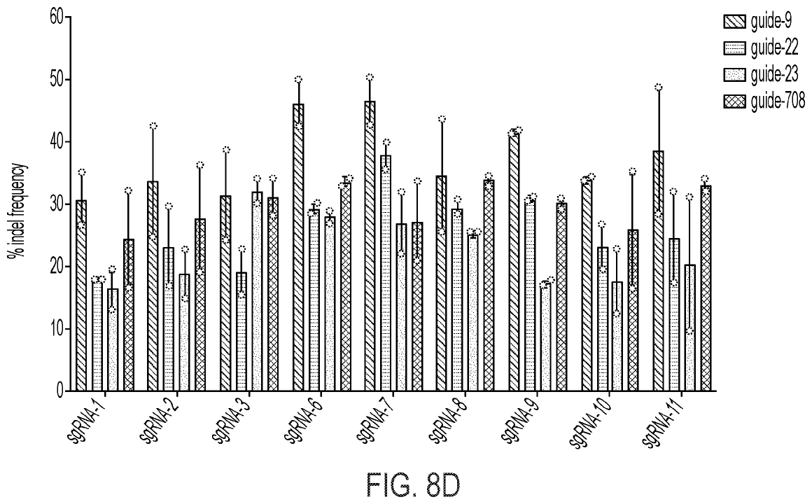

In one study, HEK293T cells were transfected with LubCas9 nuclease and different sgRNA designs (Table 8) and guide length for targeting EMX1 site 9. The targeted EMX1 site 9 had the following sequence: 5′-GTGCCCCTCCCTCCCTGGCCCAGGTG-3′ (SEQ ID NO: 100) (PAM underlined). The data for these studies are shown in FIG. 8A. FIG. 8A shows that the sgRNA-2 and sgRNA-3 designs tended to have the highest indel frequency in these assays. Specifically, sgRNA-2 and sgRNA-3 having a length of 21+G and 23+G had the highest indel frequencies of the tested sgRNAs in this assay.

In an additional study, HEK293T cells were transfected with LubCas9 nuclease and different sgRNA designs (Table 8) and guide length for targeting VEGFA site 22. The data for these studies are shown in FIG. 8B. The targeted VEGFA site 22 had the following sequence: 5′-GAGGTCAGAAATAGGGGGTCCAGGAG-3′ (SEQ ID NO: 101) (PAM underlined). The data for these studies are shown in FIG. 8B.

In an additional study, HEK293T cells were transfected with LubCas9 nuclease and different sgRNA designs (Table 8) and guide length for targeting VEGFA site 23. The data for these studies are shown in FIG. 8C. The targeted VEGFA site 23 had the following sequence: 5′-GTGCAGACGGCAGTCACTAGGGGGCG-3′ (SEQ ID NO: 102) (PAM underlined).

Another study was performed to test LubCas9 nuclease activity using various sgRNA (Table 8) and 21 nucleotide guides. For these studies, HEK293T cells were transfected with LubCas9 nuclease and different sgRNA designs targeting EMX1 site 9, VEGFA site 22, VEGFA site 23, and Hek4 site 708. The data for these experiments are shown in FIG. 8D. The Hek4 site 708 has the following sequence: 5′-GGTGGCACTGCGGCTGGAGGTGGGG-3′(SEQ ID NO: 103) (PAM underlined).

### Example 8: LubCas9 ABE and CBE Activity with Various sgRNAs and Different Guide Lengths

This example shows LubCas9 ABE activity using various sgRNAs (Table 8) and different guide lengths.

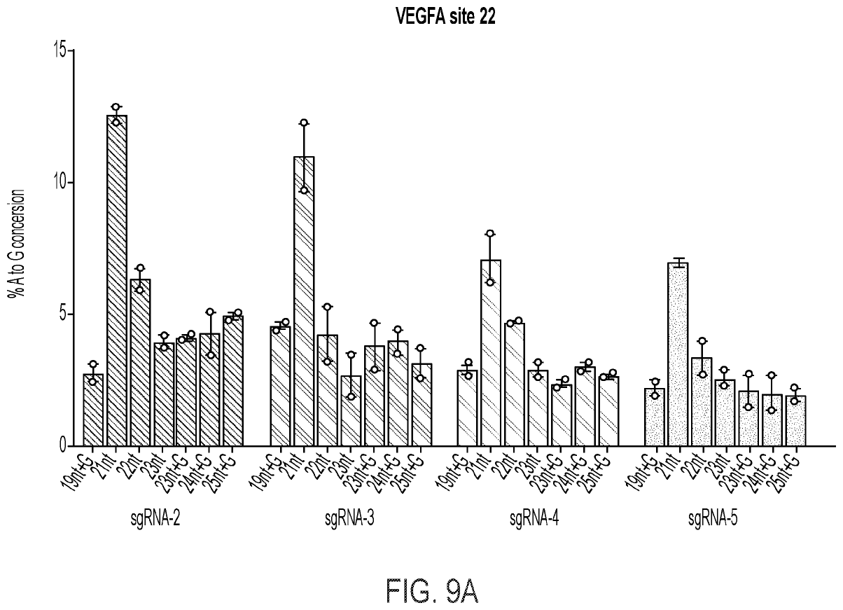

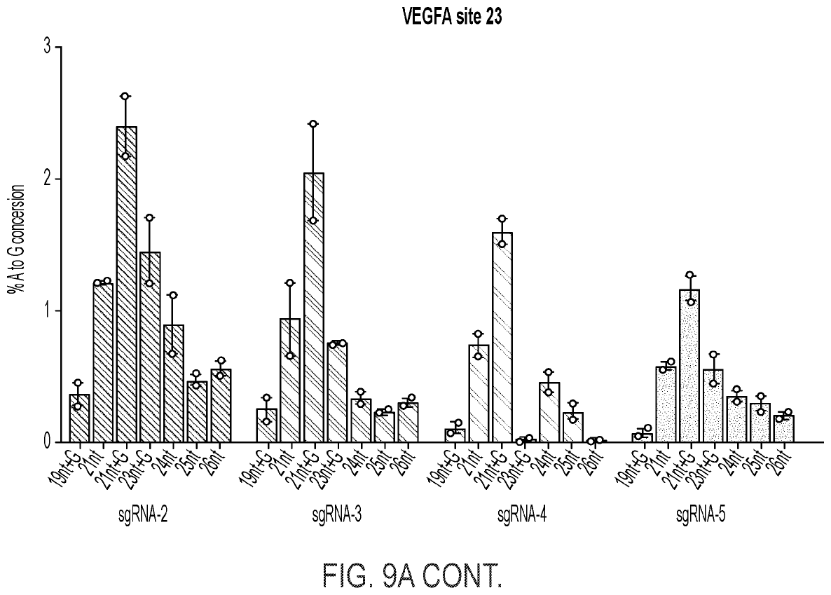

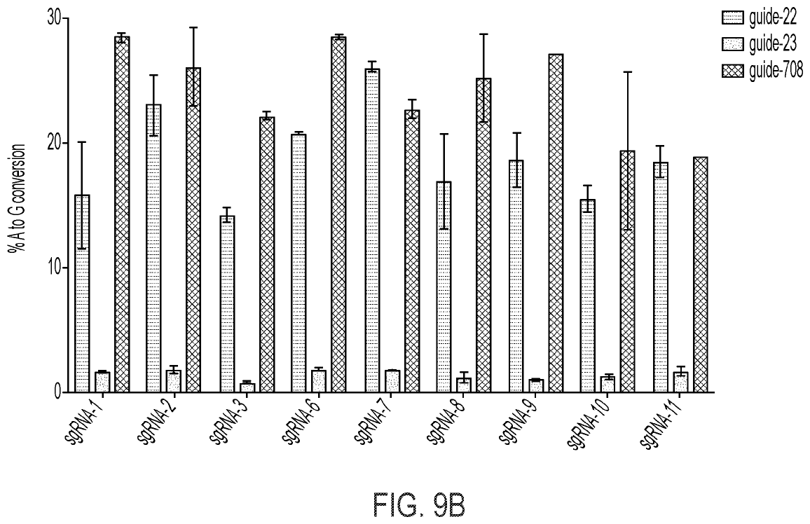

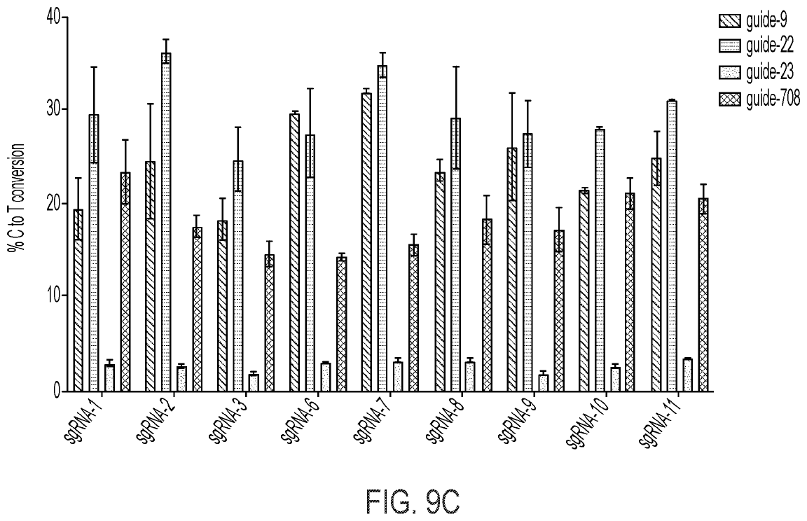

In one study, HEK293T cells were transfected with ABE-dLubCas9 and different sgRNA designs (Table 8) and guide lengths for targeting VEGFA site 22 or 23. The ABE that was used in this study was TadA*8.13. The sgRNA designs used for these studies included sgRNA-2, sgRNA-3, sgRNA-4 and sgRNA-5. The data for these studies are shown in FIG. 9A.

ABE-d-LubCas9 nuclease activity was also tested using various sgRNAs (Table 8) and 21 nucleotide guides. For these studies, HEK293T cells were transfected with ABE-dLubCas9 nuclease and different sgRNA designs targeting VEGFA site 22, VEGFA site 23 & Hek4 site 708. The ABE that was used in this study was TadA*8.13. The data for these studies are shown in FIG. 9B. The data show that the guides targeting VEGF site 22 and Hek4 site 708 had the highest amount of A-to-G conversion.

CBE-dlubCas9 nuclease activity was also tested using various sgRNAs (Table 8) and 21 nucleotide guides. For these studies, HEK293T cells were transfected with CBE-dLubCas9 nuclease and different sgRNA designs targeting EMX1 site 9, VEGFA site 22, VEGFA site 23 and Hek4 site 708. The CBE used in this study was ppAPOBEC-1 (Pongo pygmaeus): The data for these studies are shown in FIG. 9C.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above Description, but rather is as set forth in the following claims.

1. An engineered, non-naturally occurring Cas9 protein modified from Lachnospira Cas9, having at least 80% identity to
(SEQ ID NO: 1)
MSVNVGLDIGIASVGVAVVDSESGEILEAVSDLFESAEANQNVDRRGFRQSRRLKRRQY
NRIHDFMKLWEEFGFVKPENINLNTVGLRVKSLTEQVTLDELYVILLSELKHRGISYLED
SEEVDGGSEYKEGLRINQRELQSKYPCEIQLERLKIYGRYRGNFTVEIDGEKVGLSNVFT
TGAYRKEIQQLLSIQKTYQSKLTDDFINKYLEIFDRKRQYYVGPGNEKSRTDYGRYTTK
KDAEGNYITDENIFEKLIGKCSIYPEEMRAAGASYTAQEFNLLNDLNNLTIGGRKIEEEEK
RAIIETIKSSKVVNVEKIICKVTGEDAETITGARIDKDDKRIYHSFECYRKLKKALETIEVK
IEEYSREELDELARILTLNTEREGILGELEKSFLDLGEEVIDCVIDFRRKNGPLFSKWQSFS
LRLMNDIIPDMYEQPKEQMTLLTEMGLMKSKKEIFKGMKYIPENVMRDDIYNPVVVRS
VRIAVRALNAVIKKYGEIDKVVIEMPRDRNTEEQKKRIDAENKRNREELPGIEKRILEEY
GIKITSAHYRNHKQLGLKLKLWNEQGGICPYSGKTIDLERLLQNAGDYEVDHIIPLSISLD
DSRNNKVLVYASENQKKGNQTPYAYLSSVQREWGWEQYRHYVLSDLKKKKISSKKIE
NYLFMKDISKIDVVKGFIQRNLNDTRYASKVVLNTLESFFKANEKETKVSVIRGSFTSLM
RKNLKLDKSREESYAHHAVDALLIAYSKMGYDSYHKLQGEFIDFETGEILDSRMWETNL
EPDILKGYLYGRKWSEIRENIKIAESRVKYWHMTNKKCNRSLCNQTLYGTRTYDGKIYQ
IKKIKDIRTPEGLKTFKDLVDKNKGDHLLMARNDPKTYEQILQIYRDYSDAKNPFLQYE
METGDCIRKYSKKHNGSRIVSLKYHDGEVNSCIDVSHKYGFEKGSQKVVLMSLNPYRM
DVYKNCNDGKYYLIGLKQSDIKCEGRHYVIDEEKYAKVLVNEKMIQPGQSRKDLPDLG
YEFVMSFYKNEIIQYEKDGKFYKERFLSRTKPASRNYIETKPVDKPNFEKRHQIGLAKTT
FIRKIRTDILGNEYNCDREKFSSIC.

2.-5. (canceled)

6. The Cas9 protein of claim 1, wherein (i) the Cas9 protein has nickase activity, (ii) the Cas9 protein comprises at least one amino acid mutation in PAM Interacting, HNH and/or RuvC domain, (iii) the Cas9 protein further comprises a nuclear localization sequence (NLS) and/or a FLAG, HIS or HA tag, and/or (iv) the Cas9 protein recognizes a PAM sequence comprising 5′-NNGNG-3′.

7. (canceled)

8. The Cas9 protein of claim 1, wherein the amino acid sequence comprises at least one mutation in an amino acid residue, wherein (i) the mutation is D8A H593A, and/or N616A, and/or (ii) the mutation results in an inactive Cas9.

9.-11. (canceled)

12. An engineered, non-naturally occurring Cas9 fusion protein comprising a Cas9 protein having at least 80% identity to SEQ ID NO: 1, and wherein the Cas9 protein is fused to a histone demethylase, a transcriptional activator, or to a deaminase, wherein the deaminase is a cytosine deaminase or an adenosine deaminase.

13.-14. (canceled)

15. A nucleic acid encoding the Cas9 protein of claim 1.

16.-19. (canceled)

20. A method of cleaving a target nucleic acid in a eukaryotic cell comprising:
contacting the cell with a Cas9 of claim 1, and an RNA guide or a nucleic acid encoding the RNA guide, wherein the RNA guide comprises a direct repeat sequence and a spacer sequence capable of hybridizing to the target nucleic acid, and
wherein the Cas9 protein is capable of binding to the RNA guide and of causing a break in the target nucleic acid sequence complementary to the RNA guide, wherein the break is a single-stranded break or a double-stranded break.

21.-22. (canceled)

23. A method of modifying a target nucleic acid in a eukaryotic cell comprising:
contacting the cell with a Cas9 of claim 1, and an RNA guide or a nucleic acid encoding the RNA guide, wherein the RNA guide comprises a direct repeat sequence and a spacer sequence capable of hybridizing to the target nucleic acid, and wherein the Cas9 protein is capable of binding to the RNA guide and editing the target nucleic acid sequence complementary to the RNA guide.

24. The method of claim 23, wherein the Cas9 protein is an inactive Cas9 (dCas9).

25. The method of claim 24, wherein the dCas9 is fused to a deaminase.

26. The method of claim 20 or 23, wherein the RNA guide comprises a crRNA and a tracrRNA.

27. The method of claim 26, wherein the crRNA comprises a guide sequence and a direct repeat (DR) sequence of between about 16 and 26 nucleotides long.

28.-30. (canceled)

31. The method of claim 26, wherein the crRNA comprises a DR sequence comprising a sequence having at least about 80% identity to AUUUUAGUUCCUGGAUAAUUCAAGUUAGUGUAAAAC (SEQ ID NO: 3), or at least about 80% identity to AUUUUAGUUCCUGGAUAAUUCA (SEQ ID NO: 4); or comprises a sequence of
(SEQ ID NO: 5)
NNNNNNNNNNNNNNNNNNNNAUUUUAGUUCCUGGAUAAUUCA.

32.-36. (canceled)

37. The method of claim 26, wherein the tracrRNA comprises a sequence having at least about 80% identity to
(SEQ ID NO: 6)
UGAAUUAUUCAGACCAACUAAAACAAGGCUUUAUGCCGAAAUCAAGGACAC
CUUCGGGUGUCCUUUU1:JU.

38.-39. (canceled)

40. The method of claim 20 or 23, wherein the RNA guide comprises a sgRNA, wherein the sgRNA comprises a scaffold comprising a sequence having at least about 80% identity to
(SEQ ID NO: 7)
AUUUUAGUUCCUGGAUAUAAUUAUUCAGACCAACUAAAACAAGGCUUUAUG
CCGAAAUCAAGGACACCUUCGGGUGUCCUUUUUUCUUUUU;
or
(SEQ ID NO: 13)
AUUUUAGUUCCUGGAUAAUUGAAAUGAAUUAUUCAGACCAACUAAAACAAG
GCUUUAUGCCGAAAUCAAGGACACCUUCGGGUGUCCUUUUUU;
or
(SEQ ID NO: 14)
AUUUUAGUUCCUGGAUAAUUCAAAUUAUUCAGACCAACUAAAACAAGGCUU
UAUGCCGAAAUCAAGGACACCUUCGGGUGUCCUUUUUUCUUUUUAAGGAGG
AAUAG;
or
(SEQ ID NO: 15)
AUUUUAGUUCCUGGAUAAUUCAAAUUAUUCAGACCAACUAAAACAAGGCUU
UAUGCCGAAAUCAAGGACACCUUCGGGUGUCCUUUGUUCUUUAUAAGGAGC
AAUAG;
or
(SEQ ID NO: 16)
AUUUUAGUUCCUGGUAAUUCAGACCAACUAAAACAAGGCUUUAUGCCGAAA
UCAAGGACACCUUCGGGUGUCCUUUUUUCUUUUU;
or
(SEQ ID NO: 17)
AUUUUAGUUCCUGGUAAUUCAGACCAACUAAAACAAGGCUUUAUGCCGAAA
UCAAGGACACCUUCGGGUGUCCUUCUUUCUUUUU;
or
(SEQ ID NO: 18)
AUUUUAGUUCCUGGAUAAUUGAAAAAUUAUUCAGACCAACUAAAACAAGGC
UUUAUGCCGAAAUCAAGGACACCUUCGGGUGUCCUUUUUU;
or
(SEQ ID NO: 19)
AUUUUAGUUCCUGGAUAAUGAAAAUUAUUCAGACCAACUAAAACAAGGCUU
UAUGCCGAAAUCAAGGACACCUUCGGGUGUCCUUUUUU;
or
(SEQ ID NO: 20)
AUUUUAGUUCCUGGAUAAGAAAUUAUUCAGACCAACUAAAACAAGGCUUUA
UGCCGAAAUCAAGGACACCUUCGGGUGUCCUUUUUU;
or
(SEQ ID NO: 21)
AUUUUAGUUCCUGGAUAGAAAUAUUCAGACCAACUAAAACAAGGCUUUAUG
CCGAAAUCAAGGACACCUUCGGGUGUCCUUUUUU;
or
(SEQ ID NO: 22)
AUUUUAGUUCCUGGAUGAAAAUUCAGACCAACUAAAACAAGGCUUUAUGCC
GAAAUCAAGGACACCUUCGGGUGUCCUUUUUU;
or
(SEQ ID NO: 23)
AUUUUAGUUCCUGGAGAAAUUCAGACCAACUAAAACAAGGCUUUAUGCCGA
AAUCAAGGACACCUUCGGGUGUCCUUUUUU.

41.-44. (canceled)

45. The method of claim 20 or 23, wherein the target nucleic acid is 5′ to a protospacer adjacent motif (PAM) sequence, wherein the PAM has a sequence of 5′-NNGNG-3′.

46.-49. (canceled)

50. An engineered, non-naturally occurring CRISPR-Cas system comprising:
an RNA guide or a nucleic acid encoding the RNA guide, wherein the RNA guide comprises a direct repeat sequence and a spacer sequence capable of hybridizing to a target nucleic acid; and
a codon-optimized CRISPR-associated (Cas) protein having at least 80% sequence identity to SEQ ID NO: 1, and wherein the Cas protein is capable of binding to the RNA guide and (i) causing a break in the target nucleic acid sequence complementary to the RNA guide, and/or (ii) editing the target nucleic acid sequence complementary to the RNA guide, wherein the other Cas protein is fused to a deaminase.

51.-68. (canceled)

69. A vector comprising the system of claim 50, wherein the vector is a plasmid or viral vector, wherein the viral vector is an AAV vector.

70.-73. (canceled)

74. A method of treating a disorder or a disease in a subject in need thereof, the method comprising administering to the subject a system of claim 50,
wherein the guide RNA is complementary to at least 10 nucleotides of a target nucleic acid associated with the condition or disease;
wherein the Cas protein associates with the guide RNA; wherein the guide RNA binds to the target nucleic acid;
wherein (i) the Cas protein causes a break in the target nucleic acid, or (ii) the Cas9 is an inactive Cas9 (dCas9) fused to a deaminase and results in one or more base edits in the target nucleic acid, thereby treating the disorder or disease.

75.-76. (canceled)

77. A base editor comprising the fusion protein of Cas9 of claim 12, wherein (ii) the base editor comprises an adenosine deaminase domain, and wherein the one or more guide RNAs target the base editor to effect an A⋅T to G⋅C alteration in the polynucleotide or (ii) the base editor comprises a cytidine deaminase domain, and wherein the one or more guide RNAs target the base editor to effect an C⋅G to T⋅A alteration in the polynucleotide.

78. (canceled)

79. A method of editing a nucleobase of a polynucleotide, the method comprising contacting the polynucleotide with the base editor of claim 77 in complex with one or more guide RNAs, wherein the editing results in less than 50% indel formation in a target polynucleotide sequence.

80.-82. (canceled)


SEQUENCE LISTING (103 sequences)

SEQ ID NO: 1 - protein, 1097 aa, Lachnospira sp.
MSVNVGLDIGIASVGVAVVDSESGEILEAVSDLFESAEANQNVDRRGFRQSRRLKRRQYNRIHDFMKLWEEFGFVKPENINLNTVGLRVKSLTEQVTLDELYVILLSELKHRGISYLEDSEEVDGGSEYKEGLRINQRELQSKYPCEIQLERLKIYGRYRGNFTVEIDGEKVGLSNVFTTGAYRKEIQQLLSIQKTYQSKLTDDFINKYLEIFDRKRQYYVGPGNEKSRTDYGRYTTKKDAEGNYITDENIFEKLIGKCSIYPEEMRAAGASYTAQEFNLLNDLNNLTIGGRKIEEEEKRAIIETIKSSKVVNVEKIICKVTGEDAETITGARIDKDDKRIYHSFECYRKLKKALETIEVKIEEYSREELDELARILTLNTEREGILGELEKSFLDLGEEVIDCVIDFRRKNGPLFSKWQSFSLRLMNDIIPDMYEQPKEQMTLLTEMGLMKSKKEIFKGMKYIPENVMRDDIYNPVVVRSVRIAVRALNAVIKKYGEIDKVVIEMPRDRNTEEQKKRIDAENKRNREELPGIEKRILEEYGIKITSAHYRNHKQLGLKLKLWNEQGGICPYSGKTIDLERLLQNAGDYEVDHIIPLSISLDDSRNNKVLVYASENQKKGNQTPYAYLSSVQREWGWEQYRHYVLSDLKKKKISSKKIENYLFMKDISKIDVVKGFIQRNLNDTRYASKVVLNTLESFFKANEKETKVSVIRGSFTSLMRKNLKLDKSREESYAHHAVDALLIAYSKMGYDSYHKLQGEFIDFETGEILDSRMWETNLEPDILKGYLYGRKWSEIRENIKIAESRVKYWHMTNKKCNRSLCNQTLYGTRTYDGKIYQIKKIKDIRTPEGLKTFKDLVDKNKGDHLLMARNDPKTYEQILQIYRDYSDAKNPFLQYEMETGDCIRKYSKKHNGSRIVSLKYHDGEVNSCIDVSHKYGFEKGSQKVVLMSLNPYRMDVYKNCNDGKYYLIGLKQSDIKCEGRHYVIDEEKYAKVLVNEKMIQPGQSRKDLPDLGYEFVMSFYKNEIIQYEKDGKFYKERFLSRTKPASRNYIETKPVDKPNFEKRHQIGLAKTTFIRKIRTDILGNEYNCDREKFSSIC

SEQ ID NO: 2 - DNA, 3450 bp, Lachnospira sp.
atgcccaagaagaagcggaaggttggttctgtcaacgtggggctggatattggcatcgcatcagtgggagtcgccgtggtggatagtgaaagtggagagattctggaggctgtgtccgacctgttcgagtctgccgaggccaaccagaatgtggatcggagaggctttagacagagcaggcgcctgaagcggagacagtataataggatccacgacttcatgaagctgtgggaggagttcggctttgtgaagcccgagaacatcaatctgaacaccgtgggactgagggtgaagagcctgaccgagcaggtgacactggatgagctgtacgtgatcctgctgtccgagctgaagcaccggggcatcagctatctggaggactccgaggaggtggatggaggatccgagtacaaggagggactgcggatcaaccagagagagctgcagtctaagtatccttgcgagatccagctggagagactgaagatctacggccggtatagaggcaatttcaccgtggagatcgacggcgagaaagtgggcctgagcaacgtgtttaccacaggcgcctacaggaaggagatccagcagctgctgtctatccagaagacctatcagagcaagctgacagacgatttcatcaataagtacctggagatctttgacaggaagcgccagtactatgtgggcccaggcaacgagaagtcccggaccgattacggcagatataccacaaagaaggacgccgagggcaattacatcacagatgagaacatcttcgagaagctgatcggcaagtgtagcatctatccagaggagatgagggcagcaggagcatcctacaccgcccaggagtttaatctgctgaacgacctgaacaatctgacaatcggcggccggaagatcgaggaggaggagaagagagccatcatcgagaccatcaagagctccaaggtggtgaatgtggagaagatcatctgcaaggtgacaggagaggacgcagagaccatcacaggagcaaggatcgataaggacgataagcgcatctatcactccttcgagtgttacagaaagctgaagaaggccctggagaccatcgaggtgaagatcgaggagtactctagggaggagctggacgagctggcaaggatcctgaccctgaacacagagagggagggaatcctgggagagctggagaagtctttcctggatctgggcgaggaagtgatcgactgcgtgatcgacttccggcgcaagaatggccctctgttcagcaagtggcagagcttttccctgaggctgatgaacgacatcatcccagatatgtatgagcagcccaaggagcagatgaccctgctgacagagatgggcctgatgaagagcaagaaggagatctttaagggcatgaagtatatccccgagaatgtgatgagagacgatatctacaaccctgtggtggtgcggtccgtgagaatcgccgtgagggccctgaatgccgtgatcaagaagtacggcgagatcgacaaggtggtcatcgagatgcctcgggatagaaacaccgaggagcagaagaagcggatcgacgccgagaataagaggaaccgcgaggagctgccaggcatcgagaagagaatcctggaggagtatggcatcaagatcacctccgcccactacaggaatcacaagcagctgggcctgaagctgaagctgtggaacgagcagggcggcatctgtccctattctggcaagacaatcgatctggagagactgctgcagaacgccggcgactacgaggtggatcacatcatccctctgtctatcagcctggacgattctaggaacaataaggtgctggtgtacgccagcgagaatcagaagaagggcaaccagaccccctacgcctatctgtctagcgtgcagagagagtggggctgggagcagtacaggcactatgtgctgagcgacctgaagaagaagaagatctcctctaagaagatcgagaattatctgttcatgaaggacatctccaagatcgatgtggtgaagggctttatccagaggaatctgaacgatacccgctacgccagcaaggtggtgctgaatacactggagtccttctttaaggccaacgagaaggagaccaaggtgagcgtgatccgcggctccttcacatctctgatgcggaagaacctgaagctggacaagagcagggaggagtcctatgcacaccacgcagtggacgcactgctgatcgcctactccaagatgggctacgattcttatcacaagctgcagggcgagttcatcgactttgagaccggcgagatcctggatagccgcatgtgggagacaaatctggagcctgatatcctgaagggctacctgtatggccggaagtggtccgagatcagagagaacatcaagatcgccgagtctcgggtgaagtactggcacatgaccaataagaagtgcaaccggagcctgtgcaaccagacactgtacggcacccggacatatgacggcaagatctaccagatcaagaagatcaaggatatccgcaccccagagggcctgaagacattcaaggacctggtggataagaataagggcgaccacctgctgatggcccgcaacgatccaaagacctacgagcagatcctgcagatctaccgggactattctgatgccaagaatccctttctgcagtatgagatggagacaggcgactgcatcagaaagtacagcaagaagcacaatggctctaggatcgtgagcctgaagtatcacgacggcgaggtgaactcttgtatcgatgtgagccacaagtacggcttcgagaagggctcccagaaggtggtgctgatgtctctgaacccatatcggatggacgtgtacaagaattgcaacgatggcaagtactatctgatcggcctgaagcagtccgacatcaagtgtgagggccgccactatgtgatcgatgaggagaagtacgccaaggtgctggtgaatgagaagatgatccagcctggccagtctcggaaggacctgccagatctgggctatgagttcgtgatgagcttttacaagaacgagatcatccagtatgagaaggacggcaagttctacaaggagaggtttctgagccgcaccaagcccgcctcccgcaattacatcgagacaaagcccgtggataagcctaacttcgagaagcggcaccagatcggcctggccaagaccaccttcatcaggaagatccgcaccgacatcctgggcaacgaatacaactgcgatagagagaagttttcctccatctgcaaaaggccggcggccacgaaaaaggccggccaggcaaaaaagaaaaagggatcctacccatacgatgttccagattacgcttatccctacgacgtgcctgattatgcatacccatatgatgtccccgactatgcc

SEQ ID NO: 3 - RNA, 36 nt, Lachnospira sp.
auuuuaguuccuggauaauucaaguuaguguaaaac

SEQ ID NO: 4 - RNA, 22 nt, Lachnospira sp.
auuuuaguuccuggauaauuca

SEQ ID NO: 5 - RNA, 42 nt, Lachnospira sp.
  misc_feature (1)..(20): n is a, c, g, or u
nnnnnnnnnnnnnnnnnnnnauuuuaguuccuggauaauuca

SEQ ID NO: 6 - RNA, 69 nt, Lachnospira sp.
ugaauuauucagaccaacuaaaacaaggcuuuaugccgaaaucaaggacaccuucggguguccuuuuuu

SEQ ID NO: 7 - RNA, 91 nt, Lachnospira sp.
auuuuaguuccuggauauaauuauucagaccaacuaaaacaaggcuuuaugccgaaaucaaggacaccuucggguguccuuuuuucuuuuu

SEQ ID NO: 8-12 - skipped (5 entries)

SEQ ID NO: 13 - RNA, 93 nt, Lachnospira sp.
auuuuaguuccuggauaauugaaaugaauuauucagaccaacuaaaacaaggcuuuaugccgaaaucaaggacaccuucggguguccuuuuuu

SEQ ID NO: 14 - RNA, 107 nt, Lachnospira sp.
auuuuaguuccuggauaauucaaauuauucagaccaacuaaaacaaggcuuuaugccgaaaucaaggacaccuucggguguccuuuuuucuuuuuaaggaggaauag

SEQ ID NO: 15 - RNA, 107 nt, Lachnospira sp.; identical to SEQ ID NO: 14

SEQ ID NO: 16 - RNA, 85 nt, Lachnospira sp.
auuuuaguuccugguaauucagaccaacuaaaacaaggcuuuaugccgaaaucaaggacaccuucggguguccuuuuuucuuuuu

SEQ ID NO: 17 - RNA, 85 nt, Lachnospira sp.
auuuuaguuccugguaauucagaccaacuaaaacaaggcuuuaugccgaaaucaaggacaccuucggguguccuucuuucuuuuu

SEQ ID NO: 18 - RNA, 91 nt, Lachnospira sp.
auuuuaguuccuggauaauugaaaaauuauucagaccaacuaaaacaaggcuuuaugccgaaaucaaggacaccuucggguguccuuuuuu

SEQ ID NO: 19 - RNA, 89 nt, Lachnospira sp.
auuuuaguuccuggauaaugaaaauuauucagaccaacuaaaacaaggcuuuaugccgaaaucaaggacaccuucggguguccuuuuuu

SEQ ID NO: 20 - RNA, 87 nt, Lachnospira sp.
auuuuaguuccuggauaagaaauuauucagaccaacuaaaacaaggcuuuaugccgaaaucaaggacaccuucggguguccuuuuuu

SEQ ID NO: 21 - RNA, 85 nt, Lachnospira sp.
auuuuaguuccuggauagaaauauucagaccaacuaaaacaaggcuuuaugccgaaaucaaggacaccuucggguguccuuuuuu

SEQ ID NO: 22 - RNA, 83 nt, Lachnospira sp.
auuuuaguuccuggaugaaaauucagaccaacuaaaacaaggcuuuaugccgaaaucaaggacaccuucggguguccuuuuuu

SEQ ID NO: 23 - RNA, 83 nt, Lachnospira sp.; identical to SEQ ID NO: 22

SEQ ID NO: 24 - protein, 1588 aa, Lachnospira sp.
MPAAKRVKLDGTSEKGPSTGDPTLRRRIESWEFDVFYDPRELRKETCLLYEIKWGMSRKIWRSSGKNTTNHVEVNFIKKFTSERRFHSSISCSITWFLSWSPCWECSQAIREFLSQHPGVTLVIYVARLFWHMDQRNRQGLRDLVNSGVTIQIMRASEYYHCWRNFVNYPPGDEAHWPQYPPLWMMLYALELHCIILSLPPCLKISRRWQNHLAFFRLHLQNCHYQTIPPHILLATGLIHPSVTWRLKSGGSSGGSSGSETPGTSESATPESSGGSSGGSPKKKRKVGSVNVGLAIGIASVGVAVVDSESGEILEAVSDLFESAEANQNVDRRGFRQSRRLKRRQYNRIHDFMKLWEEFGFVKPENINLNTVGLRVKSLTEQVTLDELYVILLSELKHRGISYLEDSEEVDGGSEYKEGLRINQRELQSKYPCEIQLERLKIYGRYRGNFTVEIDGEKVGLSNVFTTGAYRKEIQQLLSIQKTYQSKLTDDFINKYLEIFDRKRQYYVGPGNEKSRTDYGRYTTKKDAEGNYITDENIFEKLIGKCSIYPEEMRAAGASYTAQEFNLLNDLNNLTIGGRKIEEEEKRAIIETIKSSKVVNVEKIICKVTGEDAETITGARIDKDDKRIYHSFECYRKLKKALETIEVKIEEYSREELDELARILTLNTEREGILGELEKSFLDLGEEVIDCVIDFRRKNGPLFSKWQSFSLRLMNDIIPDMYEQPKEQMTLLTEMGLMKSKKEIFKGMKYIPENVMRDDIYNPVVVRSVRIAVRALNAVIKKYGEIDKVVIEMPRDRNTEEQKKRIDAENKRNREELPGIEKRILEEYGIKITSAHYRNHKQLGLKLKLWNEQGGICPYSGKTIDLERLLQNAGDYEVDHIIPLSISLDDSRNNKVLVYASENQKKGNQTPYAYLSSVQREWGWEQYRHYVLSDLKKKKISSKKIENYLFMKDISKIDVVKGFIQRNLNDTRYASKVVLNTLESFFKANEKETKVSVIRGSFTSLMRKNLKLDKSREESYAHHAVDALLIAYSKMGYDSYHKLQGEFIDFETGEILDSRMWETNLEPDILKGYLYGRKWSEIRENIKIAESRVKYWHMTNKKCNRSLCNQTLYGTRTYDGKIYQIKKIKDIRTPEGLKTFKDLVDKNKGDHLLMARNDPKTYEQILQIYRDYSDAKNPFLQYEMETGDCIRKYSKKHNGSRIVSLKYHDGEVNSCIDVSHKYGFEKGSQKVVLMSLNPYRMDVYKNCNDGKYYLIGLKQSDIKCEGRHYVIDEEKYAKVLVNEKMIQPGQSRKDLPDLGYEFVMSFYKNEIIQYEKDGKFYKERFLSRTKPASRNYIETKPVDKPNFEKRHQIGLAKTTFIRKIRTDILGNEYNCDREKFSSICKRPAATKKAGQAKKKKGSSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 25 - protein, 208 aa, Petromyzon marinus
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAV

SEQ ID NO: 26 - protein, 145 aa, Homo sapiens
MDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKAPV

SEQ ID NO: 27 - protein, 198 aa, Homo sapiens [sequence omitted]

SEQ ID NO: 28 - protein, 198 aa, Mus musculus
MDSLLMKQKKFLYHFKNVRWAKGRHETYLCYVVKRRDSATSCSLDFGHLRNKSGCHVELLFLRYISDWDLDPGRCYRVTWFTSWSPCYDCARHVAEFLRWNPNLSLRIFTARLYFCEDRKAEPEGLRRLHRAGVQIGIMTFKDYFYCWNTFVENRERTFKAWEGLHENSVRLTRQLRRILLPLYEVDDLRDAFRMLGF

SEQ ID NO: 29 - protein, 198 aa, Canis sp.; identical to SEQ ID NO: 28

SEQ ID NO: 30 - protein, 199 aa, Bovine sp.
MDSLLKKQRQFLYQFKNVRWAKGRHETYLCYVVKRRDSPTSFSLDFGHLRNKAGCHVELLFLRYISDWDLDPGRCYRVTWFTSWSPCYDCARHVADFLRGYPNLSLRIFTARLYFCDKERKAEPEGLRRLHRAGVQIAIMTFKDYFYCWNTFVENHERTFKAWEGLHENSVRLSRQLRRILLPLYEVDDLRDAFRTLGL

SEQ ID NO: 31 - protein, 199 aa, Rattus rattus; identical to SEQ ID NO: 30

SEQ ID NO: 32 - protein, 198 aa, Canis lupus familiaris
MDSLLMKQRKFLYHFKNVRWAKGRHETYLCYVVKRRDSATSFSLDFGHLRNKSGCHVELLFLRYISDWDLDPGRCYRVTWFTSWSPCYDCARHVADFLRGYPNLSLRIFAARLYFCEDRKAEPEGLRRLHRAGVQIAIMTFKDYFYCWNTFVENREKTFKAWEGLHENSVRLSRQLRRILLPLYEVDDLRDAFRTLGL

SEQ ID NO: 33 - protein, 199 aa, Bos taurus; identical to SEQ ID NO: 30

SEQ ID NO: 34 - protein, 198 aa, Mus musculus [sequence omitted]

SEQ ID NO: 35 - protein, 229 aa, Rattus norvegicus [sequence omitted]

SEQ ID NO: 36 - protein, 229 aa, Mesocricetus auratus
MSSETGPVVVDPTLRRRIEPHEFDAFFDQGELRKETCLLYEIRWGGRHNIWRHTGQNTSRHVEINFIEKFTSERYFYPSTRCSIVWFLSWSPCGECSKAITEFLSGHPNVTLFIYAARLYHHTDQRNRQGLRDLISRGVTIRIMTEQEYCYCWRNFVNYPPSNEVYWPRYPNLWMRLYALELYCIHLGLPPCLKIKRRHQYPLTFFRLNLQSCHYQRIPPHILWATGFI

SEQ ID NO: 37 - protein, 236 aa, Pongo pygmaeus
MTSEKGPSTGDPTLRRRIESWEFDVFYDPRELRKETCLLYEIKWGMSRKIWRSSGKNTTNHVEVNFIKKFTSERRFHSSISCSITWFLSWSPCWECSQAIREFLSQHPGVTLVIYVARLFWHMDQRNRQGLRDLVNSGVTIQIMRASEYYHCWRNFVNYPPGDEAHWPQYPPLWMMLYALELHCIILSLPPCLKISRRWQNHLAFFRLHLQNCHYQTIPPHILLATGLIHPSVTWR

SEQ ID NO: 38 - protein, 236 aa, Oryctolagus cuniculus; identical to SEQ ID NO: 37

SEQ ID NO: 39 - protein, 235 aa, Monodelphis domestica
MNSKTGPSVGDATLRRRIKPWEFVAFFNPQELRKETCLLYEIKWGNQNIWRHSNQNTSQHAEINFMEKFTAERHFNSSVRCSITWFLSWSPCWECSKAIRKFLDHYPNVTLAIFISRLYWHMDQQHRQGLKELVHSGVTIQIMSYSEYHYCWRNFVDYPQGEEDYWPKYPYLWIMLYVLELHCIILGLPPCLKISGSHSNQLALFSLDLQDCHYQKIPYNVLVATGLVQPFVTWR

SEQ ID NO: 40 - protein, 224 aa, Pongo pygmaeus
MAQKEEAAAATEAASQNGEDLENLDDPEKLKELIELPPFEIVTGERLPANFFKFQFRNVEYSSGRNKTFLCYVVEAQGKGGQVQASRGYLEDEHAAAHAEEAFFNTILPAFDPALRYNVTWYVSSSPCAACADRIIKTLSKTKNLRLLILVGRLFMWEELEIQDALKKLKEAGCKLRIMKPQDFEYVWQNFVEQEEGESKAFQPWEDIQENFLYYEEKLADILK

SEQ ID NO: 41 - protein, 224 aa, Bos taurus
MAQKEEAAAAAEPASQNGEEVENLEDPEKLKELIELPPFEIVTGERLPAHYFKFQFRNVEYSSGRNKTFLCYVVEAQSKGGQVQASRGYLEDEHATNHAEEAFFNSIMPTFDPALRYMVTWYVSSSPCAACADRIVKTLNKTKNLRLLILVGRLFMWEEPEIQAALRKLKEAGCRLRIMKPQDFEYIWQNFVEQEEGESKAFEPWEDIQENFLYYEEKLADILK

SEQ ID NO: 42 - protein, 440 aa, Mus musculus
MQPQRLGPRAGMGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRPCYISVPSSSSSTLSNICLTKGLPETRFWVEGRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLRRIKESWGLQDLVNDFGNLQLGPPMS

SEQ ID NO: 43 - protein, 429 aa, Mus musculus
MGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRPCYIPVPSSSSSTLSNICLTKGLPETRFCVEGRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLRRIKESWGLQDLVNDFGNLQLGPPMS

SEQ ID NO: 44 - protein, 430 aa, Rattus rattus
MGPFCLGCSHRKCYSPIRNLISQETFKFHFKNRLRYAIDRKDTFLCYEVTRKDCDSPVSLHHGVFKNKDNIHAEICFLYWFHDKVLKVLSPREEFKITWYMSWSPCFECAEQVLRFLATHHNLSLDIFSSRLYNIRDPENQQNLCRLVQEGAQVAAMDLYEFKKCWKKFVDNGGRRFRPWKKLLTNFRYQDSKLQEILRPCYIPVPSSSSSTLSNICLTKGLPETRFCVERRRVHLLSEEEFYSQFYNQRVKHLCYYHGVKPYLCYQLEQFNGQAPLKGCLLSEKGKQHAEILFLDKIRSMELSQVIITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLHRIKESWGLQDLVNDFGNLQLGPPMS

SEQ ID NO: 45 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 46 - protein, 373 aa, Homo sapiens
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 47 - protein, 370 aa, Rhesus macaque
MVEPMDPRTFVSNFNNRPILSGLNTVWLCCEVKTKDPSGPPLDAKIFQGKVYSKAKYHPEMRFLRWFHKWRQLHHDQEYKVTWYVSWSPCTRCANSVATFLAKDPKVTLTIFVARLYYFWKPDYQQALRILCQKRGGPHATMKIMNYNEFQDCWNKFVDGRGKPFKPRNNLPKHYTLLQATLGELLRHLMDPGTFTSNFNNKPWVSGQHETYLCYKVERLHNDTWVPLNQHRGFLRNQAPNIHGFPKGRHAELCFLDLIPFWKLDGQQYRVTCFTSWSPCFSCAQEMAKFISNNEHVSLCIFAARIYDDQGRYQEGLRALHRDGAKIAMMNYSEFEYCWDTFVDRQGRPFQPWDGLDEHSQALSGRLRAI

SEQ ID NO: 48 - protein, 384 aa, Chimpanzee
MKPHFRNPVERMYQDTFSDNFYNRPILSHRNTVWLCYEVKTKGPSRPPLDAKIFRGQVYSKLKYHPEMRFFHWFSKWRKLHRDQEYEVTWYISWSPCTKCTRDVATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTSNFNNELWVRGRHETYLCYEVERLHNDTWVLLNQRRGFLCNQAPHKHGFLEGRHAELCFLDVIPFWKLDLHQDYRVTCFTSWSPCFSCAQEMAKFISNNKHVSLCIFAARIYDDQGRCQEGLRTLAKAGAKISIMTYSEFKHCWDTFVDHQGCPFQPWDGLEEHSQALSGRLRAILQNQGN

SEQ ID NO: 49 - protein, 377 aa, Green monkey
MNPQIRNMVEQMEPDIFVYYFNNRPILSGRNTVWLCYEVKTKDPSGPPLDANIFQGKLYPEAKDHPEMKFLHWFRKWRQLHRDQEYEVTWYVSWSPCTRCANSVATFLAEDPKVTLTIFVARLYYFWKPDYQQALRILCQERGGPHATMKIMNYNEFQHCWNEFVDGQGKPFKPRKNLPKHYTLLHATLGELLRHVMDPGTFTSNFNNKPWVSGQRETYLCYKVERSHNDTWVLLNQHRGFLRNQAPDRHGFPKGRHAELCFLDLIPFWKLDDQQYRVTCFTSWSPCFSCAQKMAKFISNNKHVSLCIFAARIYDDQGRCQEGLRTLHRDGAKIAVMNYSEFEYCWDTFVDRQGRPFQPWDGLDEHSQALSGRLRAI

SEQ ID NO: 50 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 51 - protein, 373 aa, Homo sapiens; identical to SEQ ID NO: 46

SEQ ID NO: 52 - protein, 382 aa, Homo sapiens
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVTIMDYEEFAYCWENFVYNEGQQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 53 - protein, 395 aa, Rattus rattus
MQPQGLGPNAGMGPVCLGCSHRRPYSPIRNPLKKLYQQTFYFHFKNVRYAWGRKNNFLCYEVNGMDCALPVPLRQGVFRKQGHIHAELCFIYWFHDKVLRVLSPMEEFKVTWYMSWSPCSKCAEQVARFLAAHRNLSLAIFSSRLYYYLRNPNYQQKLCRLIQEGVHVAAMDLPEFKKCWNKFVDNDGQPFRPWMRLRINFSFYDCKLQEIFSRMNLLREDVFYLQFNNSHRVKPVQNRYYRRKSYLCYQLERANGQEPLKGYLLYKKGEQHVEILFLEKMRSMELSQVRITCYLTWSPCPNCARQLAAFKKDHPDLILRIYTSRLYFWRKKFQKGLCTLWRSGIHVDVMDLPQFADCWTNFVNPQRPFRPWNELEKNSWRIQRRLRRIKESWGL

SEQ ID NO: 54 - protein, 227 aa, Bos taurus
MDGWEVAFRSGTVLKAGVLGVSMTEGWAGSGHPGQGACVWTPGTRNTMNLLREVLFKQQFGNQPRVPAPYYRRKTYLCYQLKQRNDLTLDRGCFRNKKQRHAERFIDKINSLDLNPSQSYKIICYITWSPCPNCANELVNFITRNNHLKLEIFASRLYFHWIKSFKMGLQDLQNAGISVAVMTHTEFEDCWEQFVDNQSRPFQPWDKLEQYSASIRRRLQRILTAPI

SEQ ID NO: 55 - skipped

SEQ ID NO: 56 - protein, 490 aa, Chimpanzee
MNPQIRNPMEWMYQRTFYYNFENEPILYGRSYTWLCYEVKIRRGHSNLLWDTGVFRGQMYSQPEHHAEMCFLSWFCGNQLSAYKCFQITWFVSWTPCPDCVAKLAKFLAEHPNVTLTISAARLYYYWERDYRRALCRLSQAGARVKIMDDEEFAYCWENFVYNEGQPFMPWYKFDDNYAFLHRTLKEIIRHLMDPDTFTFNFNNDPLVLRRHQTYLCYEVERLDNGTWVLMDQHMGFLCNEAKNLLCGFYGRHAELRFLDLVPSLQLDPAQIYRVTWFISWSPCFSWGCAGQVRAFLQENTHVRLRIFAARIYDYDPLYKEALQMLRDAGAQVSIMTYDEFEYCWDTFVYRQGCPFQPWDGLEEHSQALSGRLRAILQVRASSLCMVPHRPPPPPQSPGPCLPLCSEPPLGSLLPTGRPAPSLPFLLTASFSFPPPASLPPLPSLSLSPGHLPVPSFHSLTSCSIQPPCSSRIRETEGWASVSKEGRDLG

SEQ ID NO: 57 - protein, 190 aa, Homo sapiens
MNPQIRNPMKAMYPGTFYFQFKNLWEANDRNETWLCFTVEGIKRRSVVSWKTGVFRNQVDSETHCHAERCFLSWFCDDILSPNTKYQVTWYTSWSPCPDCAGEVAEFLARHSNVNLTIFTARLYYFQYPCYQEGLRSLSQEGVAVEIMDYEDFKYCWENFVYNDNEPFKPWKGLKTNFRLLKRRLRESLQ

SEQ ID NO: 58 - protein, 190 aa, Gorilla gorilla
MNPQIRNPMKAMYPGTFYFQFKNLWEANDRNETWLCFTVEGIKRRSVVSWKTGVFRNQVDSETHCHAERCFLSWECDDILSPNTNYQVTWYTSWSPCPECAGEVAEFLARHSNVNLTIFTARLYYFQDTDYQEGLRSLSQEGVAVKIMDYKDFKYCWENFVYNDDEPFKPWKGLKYNFRFLKRRLQEILE

SEQ ID NO: 59 - protein, 199 aa, Homo sapiens; identical to SEQ ID NO: 45

SEQ ID NO: 60 - protein, 202 aa, Rhesus macaque
MDGSPASRPRHLMDPNTFTFNFNNDLSVRGRHQTYLCYEVERLDNGTWVPMDERRGFLCNKAKNVPCGDYGCHVELRFLCEVPSWQLDPAQTYRVTWFISWSPCFRRGCAGQVRVFLQENKHVRLRIFAARIYDYDPLYQEALRTLRDAGAQVSIMTYEEFKHCWDTFVDRQGRPFQPWDGLDEHSQALSGRLRAILQNQGN

SEQ ID NO: 61 - protein, 185 aa, Bos taurus
MDEYTFTENFNNQGWPSKTYLCYEMERLDGDATIPLDEYKGFVRNKGLDQPEKPCHAELYFLGKIHSWNLDRNQHYRLTCFISWSPCYDCAQKLTTFLKENHHISLHILASRIYTHNRFGCHQSGLCELQAAGARITIMTFEDFKHCWETFVDHKGKPFQPWEGLNVKSQALCTELQAILKTQQN

SEQ ID NO: 62 - protein, 200 aa, Homo sapiens
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPKFADCWENFVDHEKPLSFNPYKMLEELDKNSRAIKRRLERIKIPGVRAQGRYMDILCDAEV

SEQ ID NO: 63 - protein, 210 aa, Rhesus macaque
MALLTAKTFSLQFNNKRRVNKPYYPRKALLCYQLTPQNGSTPTRGHLKNKKKDHAEIRFINKIKSMGLDETQCYQVTCYLTWSPCPSCAGELVDFIKAHRHLNLRIFASRLYYHWRPNYQEGLLLLCGSQVPVEVMGLPEFTDCWENFVDHKEPPSFNPSEKLEELDKNSQAIKRRLERIKSRSVDVLENGLRSLQLGPVTPSSSIRNSR

SEQ ID NO: 64 - protein, 386 aa, Homo sapiens
MNPQIRNPMERMYRDTFYDNFENEPILYGRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRLHKAGARVKIMDYEDFAYCWENFVCNEGQPFMPWYKFDDNYASLHRTLKEILRNPMEAMYPHIFYFHFKNLLKACGRNESWLCFTMEVTKHHSAVFRKRGVFRNQVDPETHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDDEPFKPWKGLQTNFRLLKRRLREILQ

SEQ ID NO: 65 - protein, 236 aa, Homo sapiens
MTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 66 - protein, 229 aa, Mus musculus
MSSETGPVAVDPTLRRRIEPHEFEVFFDPRELRKETCLLYEINWGGRHSVWRHTSQNTSNHVEVNFLEKFTTERYFRPNTRCSITWFLSWSPCGECSRAITEFLSRHPYVTLFIYIARLYHHTDQRNRQGLRDLISSGVTIQIMTEQEYCYCWRNFVNYPPSNEAYWPRYPHLWVKLYVLELYCIILGLPPCLKILRRKQPQLTFFTITLQTCHYQRIPPHLLWATGLK

SEQ ID NO: 67 - protein, 229 aa, Rattus rattus [sequence omitted]

SEQ ID NO: 68 - protein, 224 aa, Homo sapiens
MAQKEEAAVATEAASQNGEDLENLDDPEKLKELIELPPFEIVTGERLPANFFKFQFRNVEYSSGRNKTFLCYVVEAQGKGGQVQASRGYLEDEHAAAHAEEAFFNTILPAFDPALRYNVTWYVSSSPCAACADRIIKTLSKTKNLRLLILVGRLFMWEEPEIQAALKKLKEAGCKLRIMKPQDFEYVWQNFVEQEEGESKAFQPWEDIQENFLYYEEKLADILK

SEQ ID NO: 69 - protein, 224 aa, Mus musculus
MAQKEEAAEAAAPASQNGDDLENLEDPEKLKELIDLPPFEIVTGVRLPVNFFKFQFRNVEYSSGRNKTFLCYVVEVQSKGGQAQATQGYLEDEHAGAHAEEAFFNTILPAFDPALKYNVTWYVSSSPCAACADRILKTLSKTKNLRLLILVSRLFMWEEPEVQAALKKLKEAGCKLRIMKPQDFEYIWQNFVEQEEGESKAFEPWEDIQENFLYYEEKLADILK

SEQ ID NO: 70 - protein, 224 aa, Rattus rattus
MAQKEEAAEAAAPASQNGDDLENLEDPEKLKELIDLPPFEIVTGVRLPVNFFKFQFRNVEYSSGRNKTFLCYVVEAQSKGGQVQATQGYLEDEHAGAHAEEAFFNTILPAFDPALKYNVTWYVSSSPCAACADRILKTLSKTKNLRLLILVSRLFMWEEPEVQAALKKLKEAGCKLRIMKPQDFEYLWQNFVEQEEGESKAFEPWEDIQENFLYYEEKLADILK

SEQ ID NO: 71 - protein, 224 aa, Bos taurus; identical to SEQ ID NO: 41

SEQ ID NO: 72 - protein, 208 aa, Petromyzon marinus
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSFMIQVKILHTTKSPAV

SEQ ID NO: 73 - protein, 381 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKFNYDEFQHCWSKFVYSQRELFEPWNNLPKYYILLHFMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKKHVSLCIFTARIYRRQGRCQEGLRTLAEAGAKISFTYSEFKHCWDTFVDHQGCPFQPWDGLDEHSQDLSGRLRAILQNQEN

SEQ ID NO: 74 - protein, 295 aa, Homo sapiens
MDPPTFTFNFNNEPWWGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFMDPPTFTFNFNNEPWWGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISFTYSEFKHCWDTFVDHQGCPFQPWDGLDEHSQDLSGRLRAILQ

SEQ ID NO: 75 - protein, 184 aa, Homo sapiens
MDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYRRQGRCQEGLRTLAEAGAKISFMTYSEFKHCWDTFVDHQGCPFQPWDGLDEHSQDLSGRLRAILQ

SEQ ID NO: 76 - protein, 367 aa, Homo sapiens
MEPIYEEYLANHGTIVKPYYWLSFSLDCSNCPYHIRTGEEARVSLTEFCQIFGFPYGTTFPQTKHLTFYELKTSSGSLVQKGHASSCTGNYIHPESMLFEMNGYLDSAIYNNDSIRHIILYSNNSPCNEANHCCISKMYNFLITYPGITLSIYFSQLYHTEMDFPASAWNREALRSLASLWPRVVLSPISGGIWHSVLHSFISGVSGSHVFQPILTGRALADRHNAYEINAITGVKPYFTDVLLQTKRNPNTKAQEALESYPLNNAFPGQFFQMPSGQLQPNLPPDLRAPVVFVLVPLRDLPPMHMGQNPNKPRNIVRHLNMPQMSFQETKDLGRLPTGRSVEIVEITEQFASSKEADEKKKKKGKK

SEQ ID NO: 77 - protein, 198 aa, Mus musculus; identical to SEQ ID NO: 28

SEQ ID NO: 78 - protein, 388 aa, Rattus norvegicus
MEPLYEEYLTHSGTIVKPYYWLSVSLNCTNCPYHIRTGEEARVPYTEFHQTFGFPWSTYPQTKHLTFYELRSSSGNLIQKGLASNCTGSHTHPESMLFERDGYLDSLIFHDSNIRHIILYSNNSPCDEANHCCISKMYNFLMNYPEVTLSVFFSQLYHTENQFPTSAWNREALRGLASLWPQVTLSAISGGIWQSILETFVSGISEGLTAVRPFTAGRTLTDRYNAYEINCITEVKPYFTDALHSWQKENQDQKVWAASENQPLHNTTPAQWQPDMSQDCRTPAVFMLVPYRDLPPIHVNPSPQKPRTVVRHLNTLQLSASKVKALRKSPSGRPVKKEEARKGSTRSQEANETNKSKWKKQTLFIKSNICHLLEREQKKIGILSSWSV

SEQ ID NO: 79 - protein, 363 aa, Macaca fascicularis
MEPTYEEYLANHGTIVKPYYWLSFSLDCSNCPYHIRTGEEARVSLTEFCQIFGFPYGTTYPQTKHLTFYELKTSSGSLVQKGHASSCTGNYIHPESMLFEMNGYLDSAIYNNDSIRHIILYCNNSPCNEANHCCISKVYNFLITYPGITLSIYFSQLYHTEMDFPASAWNREALRSLASLWPRVVLSPISGGIWHSVLHSFVSGVSGSHVFQPILTGRALTDRYNAYEINAITGVKPFFTDVLLHTKRNPNTKAQMALESYPLNNAFPGQSFQMTSGIPPDLRAPVVFVLLPLRDLPPMHMGQDPNKPRNIIRHLNMPQMSFQETKDLERLPTRRSVETVEITERFASSKQAEEKTKKKKGKK

SEQ ID NO: 80 - protein, 224 aa, Petromyzon marinus
MAGYECVRVSEKLDFDTFEFQFENLHYATERHRTYVIFDVKPQSAGGRSRRLWGYIINNPNVCHAELILMSMIDRHLESNPGVYAMTWYMSWSPCANCSSKLNPWLKNLLEEQGHTLTMHFSRIYDRDREGDHRGLRGLKHVSNSFRMGVVGRAEVKECLAEYVEASRRTLTWLDTTESMAAKMRRKLFCILVRCAGMRESGIPLHLFTLQTPLLSGRVVWWRV

SEQ ID NO: 81 - protein, 331 aa, Petromyzon marinus
MELREVVDCALASCVRHEPLSRVAFLRCFAAPSQKPRGTVILFYVEGAGRGVTGGHAVNYNKQGTSIHAEVLLLSAVRAALLRRRRCEDGEEATRGCTLHCYSTYSPCRDCVEYIQEFGASTGVRVVIHCCRLYELDVNRRRSEAEGVLRSLSRLGRDFRLMGPRDAIALLLGGRLANTADGESGASGNAWVTETNVVEPLVDMTGFGDEDLHAQVQRNKQIREAYANYASAVSLMLGELHVDPDKFPFLAEFLAQTSVEPSGTPRETRGRPRGASSRGPEIGRQRPADFERALGAYGLFLHPRIVSREADREEIKRDLIVVMRKHNYQGP

SEQ ID NO: 82 - protein, 209 aa, Petromyzon marinus
MAGDENVRVSEKLDFDTFEFQFENLHYATERHRTYVIFDVKPQSAGGRSRRLWGYIINNPNVCHAELILMSMIDRHLESNPGVYAMTWYMSWSPCANCSSKLNPWLKNLLEEQGHTLMMHFSRIYDRDREGDHRGLRGLKHVSNSFRMGVVGRAEVKECLAEYVEASRRTLTWLDTTESMAAKMRRKLFCILVRCAGMRESGMPLHLFT

SEQ ID NO: 83 - protein, 158 aa, Saccharomyces cerevisiae
MVTGGMASKWDQKGMDIAYEEAALGYKEGGVPIGGCLINNKDGSVLGRGHNMRFQKGSATLHGEISTLENCGRLEGKVYKDTTLYTTLSPCDMCTGAIIMYGIPRCVVGENVNFKSKGEKYLQTRGHEVVVVDDERCKKIMKQFIDERPQDWFEDIGE

SEQ ID NO: 84 - protein, 218 aa, Recombinant protein
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRGLPPCLNILRRKQPQLTFFTIALQSCHYQRLPPHILWATGLK

SEQ ID NO: 85 - protein, 218 aa, Recombinant protein
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQHYQRLPPHILWATGLK

SEQ ID NO: 86 - protein, 429 aa, Mus musculus; identical to SEQ ID NO: 43

SEQ ID NO: 87 - protein, 167 aa, Mus musculus
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 88 - DNA, 23 bp, Homo sapiens
tagaaccctctggggaccgtttg

SEQ ID NO: 89 - DNA, 23 bp, Homo sapiens
cctgtcaagtggcgtgacaccgg

SEQ ID NO: 90 - DNA, 23 bp, Homo sapiens
tttcccttcagctaaaataaagg

SEQ ID NO: 91 - DNA, 23 bp, Homo sapiens
cattatatcaaatctaccactgt

SEQ ID NO: 92 - DNA, 23 bp, Homo sapiens
ctgtgcccctccctccctggccc

SEQ ID NO: 93 - DNA, 23 bp, Homo sapiens
gacaaagtacaaacggcagaagc

SEQ ID NO: 94 - DNA, 23 bp, Homo sapiens
agggctcccatcacatcaaccgg

SEQ ID NO: 95 - RNA, 23 nt, Homo sapiens
gggcaaccacaaacccacgaggg

SEQ ID NO: 96 - DNA, 23 bp, Homo sapiens
gtgtgcagacggcagtcactagg

SEQ ID NO: 97 - DNA, 23 bp, Homo sapiens
cccccttcaatattcctagcaaa

SEQ ID NO: 98 - DNA, 26 bp, Homo sapiens
gtgcccctccctccctggcccaggtg

SEQ ID NO: 99 - DNA, 26 bp, Homo sapiens
gaggtcagaaatagggggtccaggag

SEQ ID NO: 100 - DNA, 26 bp, Homo sapiens
gtgcagacggcagtcactagggggcg

SEQ ID NO: 101 - DNA, 25 bp, Homo sapiens
ggtggcactgcggctggaggtgggg

SEQ ID NO: 102 - DNA, 23 bp, Homo sapiens
tgcagagcaaataccagagataa

SEQ ID NO: 103 - DNA, 23 bp, Homo sapiens
gggaggtcagaaatagggggtcc